(19) United States
(12) Patent Application Publication
(10) Pub. No.: US 2009/0076032 A1
(43) Pub. Date: Mar. 19, 2009

(54) Derivatives of 18Beta-Glycyrrhetinic Acid

(76) Inventors: Simon Ward, Chesterfield (GB); Alice Macgowan, Sheffield (GB); Stanley Roberts, Kersbrook (GB); Jenny Littlechild, Tiverton (GB); Kirsty Line, Tiverton (GB); Ed Irving, Winsford (GB); Sam Donnelly, Audenshaw (GB)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/207,899

(22) Filed: Sep. 10, 2008

Related U.S. Application Data
(63) Continuation of application No. PCT/GB2007/050118, filed on Mar. 9, 2007.

(30) Foreign Application Priority Data
Mar. 10, 2006 (GB) 0604899.5
Dec. 15, 2006 (GB) 0624963.5

(51) Int. Cl. A61K 31/4965; C07C 69/757; A61K 31/215; C07C 69/76; C07D 241/24; C07C 69/73

(52) U.S. Cl. 514/255.06; 560/116; 514/510; 560/174; 544/406; 560/84

(57) ABSTRACT

The present invention relates to novel derivatives of 18β-glycyrrhetinic acid and methods of synthesising the derivatives. Also included within the scope of the present invention are pharmaceutical compositions comprising the derivatives of the present invention and medical uses of the derivatives, including their use in inhibiting enzymes such as retinol dehydrogenases. The present invention also relates to methods of treating diseases, such as hyperproliferative diseases, neoplasms, cancers and photoageing.

### TECHNICAL FIELD

The present invention relates to novel derivatives of 18β-glycyrrhetinic acid and methods of synthesising the derivatives. Also included within the scope of the present invention are pharmaceutical compositions comprising the derivatives of the present invention and medical uses of the derivatives, including their use in inhibiting enzymes such as retinol dehydrogenases. The present invention also relates to methods of treating diseases, such as hyperproliferative diseases, neoplasms, cancers and photoageing.

### BACKGROUND ART

A number of diseases are associated with hyperproliferation of cells, including psoriasis, the ichthyoses, cancer and cutaneous viral infections. Psoriasis is a chronic inflammatory disease characterised by hyperproliferation and impaired differentiation of keratinocytes. Currently, the symptoms of psoriasis are treated in a number of ways, including topical administration of retinoids to the patient. Other diseases such as acne vulgaris and photoageing also respond to retinoid therapy and are believed to involve additional retinoid-mediated mechanisms. Retinoids have also been used for both treatment and prevention of the development of cancers (e.g. treatment of acute promyelocytic leukaemia and prevention of the development of cutaneous malignancies in renal transplant patients).

Current retinoid therapy is based upon the effect of carboxylic acid derivatives of vitamin A (in particular, retinoic acid, the endogenously active compound), which are able to transcriptionally regulate target genes. Exposure to retinoic acid and retinoids results in proliferating cells withdrawing from the cell cycle and differentiating in response to retinoic acid-induced transcription of a possible excess of 300 target genes. This is a “forced” differentiation that represents a reprogramming of the normal cell fate and can be considered as an instructive differentiation. Treatment with retinoids has been found to be effective in controlling psoriasis, turnouts and in relieving the symptoms of photoageing.

However, despite the beneficial effects of tetinoid treatment, its benefits are limited by potentially serious adverse effects including hepatotoxicity, hyperlipidaemia, inhibition of bone growth, cutaneous irritation, photosensitivity, alopecia and tetatogenicity (Kemmett and Hunter, Hospital Update, March 1988, pp 1301-1313). These effects are related to the pharmacological dosages needed to achieve a therapeutic response. To date, the search for alternative retinoids and methods of using retinoids has produced only marginal reductions in the adverse effects.

More recently, it has been suggested that the desirable physiological effects of retinoids on hyperproliferative cells, i.e. reduced proliferation and/or enhanced differentiation as well as reversal of the effects of photoageing, can also be achieved by reducing the endogenous level of retinoic acid in hyperproliferative cells of cells suffering from photoageing (WO02/15920, which is hereby incorporated in its entirety). Reduction of the endogenous level of retinoic acid in cells may be achieved in various ways, for example, by blocking the activity of a retinol binding protein receptor (RBPr) which transports retinol into the cell, or by inhibiting any of the enzymatically catalysed reactions in a pathway responsible for retinoic acid biosynthesis. Thus, the native retinoid metabolic pathway may be targeted as a means of reducing endogenous retinoid levels.

The first, and rate limiting, step in retinoic acid biosynthesis involves the oxidation of retinol to retinal by means of a retinol dehydrogenase. The retinal is then further oxidised to retinoic acid by a retinal dehydrogenase. Known inhibitors of retinol dehydrogenase include carbenoxolone, phenylarsine and citral (WO02/15920). Carbenoxolone is the 3-O-hemisuccinate derivative of 18β-glycyrrhetinic acid. 18β-Glycyrrhetinic acid (also called 18β-glycyrrhetic acid or enoxolone) and its derivatives 18β-glycyrrhizic acid (also called glycyrrhizin) and carbenoxolone have the following structures:

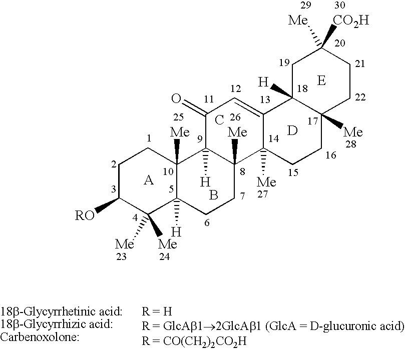

18β-Glycyrrhetinic acid and 18β-glycyrrhizic acid are naturally occurring compounds, which have been isolated from liquorice root (Glygyrrhiza uralensis). The applications of these molecules are many and varied and include, for example, their use as anti-ulcer, anti-inflammatory, anti-hormonal and anti-neoplastic agents as well as their use as commercial sweetening agents (Farina et al, Il Farmaco, 1998, 53, pp 22-32).

The wide-ranging biological activities of these compounds have led to a number of derivatives of 18β-glycyrrhetinic acid being prepared, one of the earliest of which was the 3-O-hemisuccinate derivative carbenoxolone. The disodium salt of carbenoxolone has been widely used in Europe for the treatment of peptic ulcer, however, adverse side effects such as pseudo-aldosteronism, sodium ion retention and hypertension have resulted in the drug falling out of favour in more recent years. Therefore, there is a clear need in the art for further retinol dehydrogenase inhibitors.

### SUMMARY OF THE INVENTION

A first aspect of this invention provides a compound having the formula I:

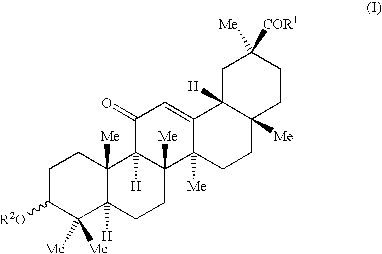

or a pharmaceutically acceptable salt thereof, wherein

R¹ is —OR^{a} or —N(R^{a})₂;

R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;

R² is

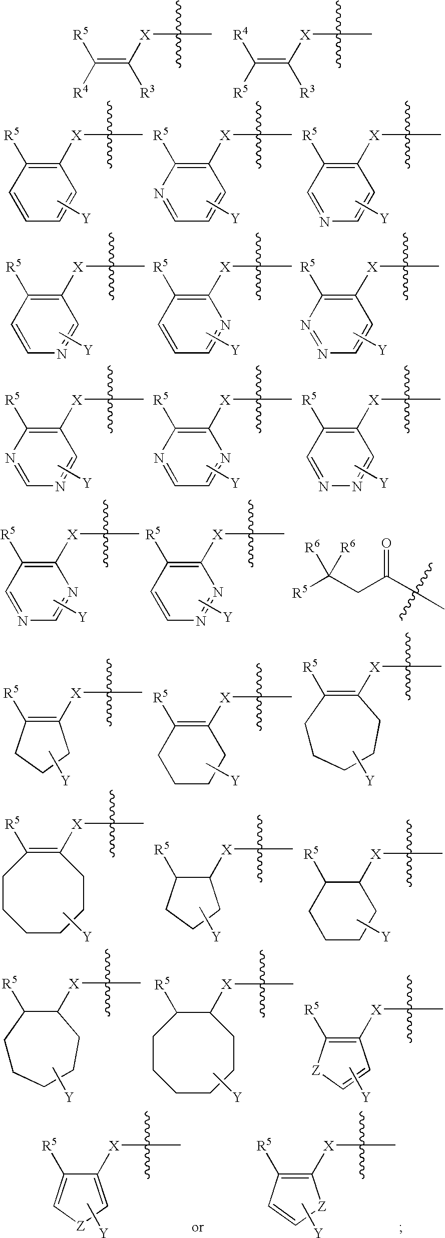

R³ and R⁴ are independently hydrogen, or an unsubstituted, straight-chained or branched alkyl group which contains 1, 2, 3 or 4 carbon atoms;

R⁵ is —OH, —CO₂H, —CO₂R⁶, —SO₃H, or —PO₃H₂;

R⁶ is an unsubstituted, straight-chained or branched alkyl group which contains 1, 2, 3 or 4 carbon atoms;

X is —CO— or —CH₂—;

Y is —H, —F, —Cl, —Br, —I, -Me, or —OMe; and

Z is —NH—, —O—, or —S—.

In a preferred embodiment, the compound of the present invention is not that compound wherein:

(a) R¹ is —ONa, and R² is

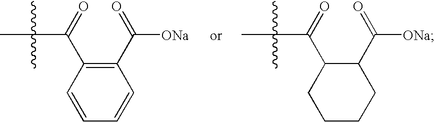

and/or

(b) R¹ is —OMe, and R² is

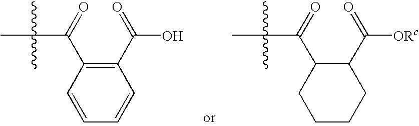

where R^{c} is H or Me; and/or

(c) R¹ is —OH, and R² is

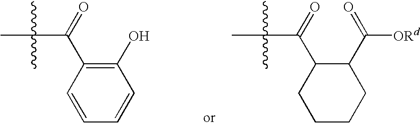

where R^{d} is H or “hexyl; and/or

(d) R¹ is —O-″hexyl, and R² is

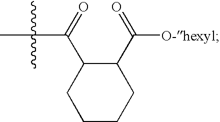

and/or

(e) R¹ is —OH or

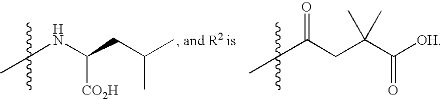

In another embodiment, the compound of the present invention is not that compound, wherein R¹ is —OH, and R² is

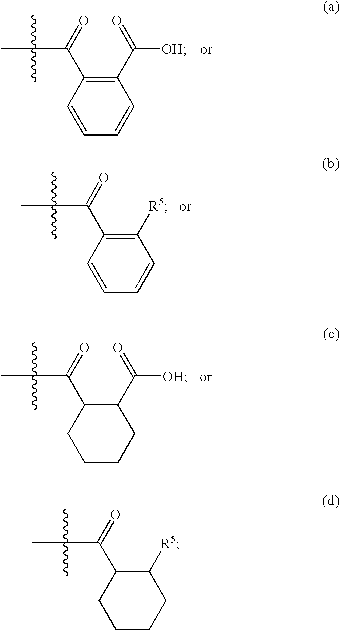

or a salt thereof (such as a sodium or disodium salt); and/or an ester thereof (such as a methyl ester or an “hexyl ester); and
wherein R⁵ is as defined above.

In another embodiment, the compound of the present invention is not that compound, wherein R¹ is —OR^{a}, and R² is

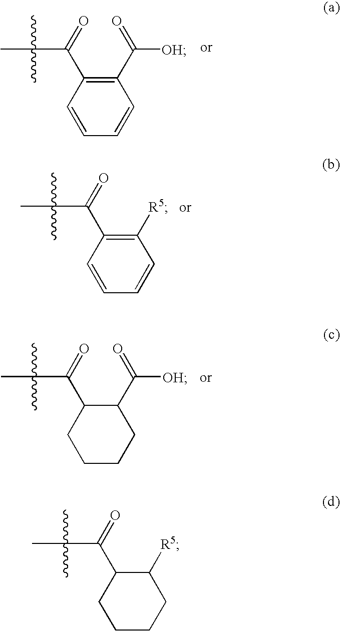

or a salt thereof (such as a sodium or disodium salt); and/or an ester thereof (such as a methyl ester or an “hexyl ester); and

wherein R^{a} and R⁵ are as defined above.

In another embodiment, the compound of the present invention is not that compound, wherein R² is

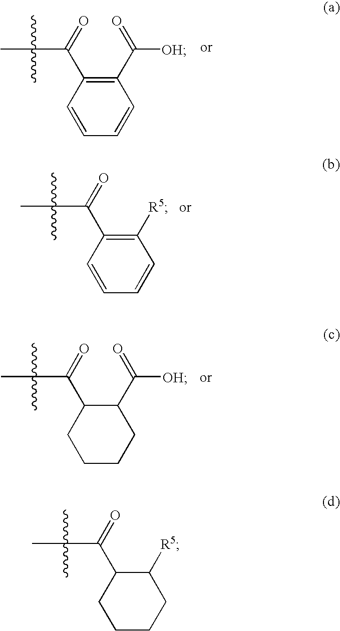

or a salt thereof (such as a sodium or disodium salt); and/or an ester thereof (such as a methyl ester or an “hexyl ester); and
wherein R⁵ is as defined above.

In another embodiment, the compound of the present invention is not that compound, wherein R¹ is —OH, —ONa, —OMe or —O-“hexyl, and R² is

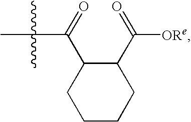

wherein R^{e} is H, Na, Me or “hexyl, or wherein R^{e} is H, Na or C₁₋₆ alkyl, or wherein R^{e} is H, Na or alkyl.

In another embodiment, the compound of the present invention is not that compound, wherein R² is

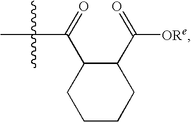

wherein R^{e} is H, Na, Me or “hexyl, or wherein R^{e} is H, Na or C₁₋₆ alkyl, or wherein R^{e} is H, Na or alkyl.

In another embodiment, the compound of the present invention is not that compound, wherein R¹ is —OH, and R² is

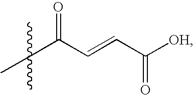

wherein the double bond of the R² side chain is in the trans configuration, or a salt thereof and/or a methyl ester thereof.

In another embodiment, the compound of the present invention is not that compound, wherein R² is

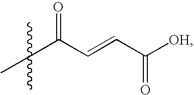

wherein the double bond of the R² side chain is in the trans configuration, or a salt thereof and/or a methyl ester thereof.

In another embodiment, the compound of the present invention is not that compound, wherein R¹ is

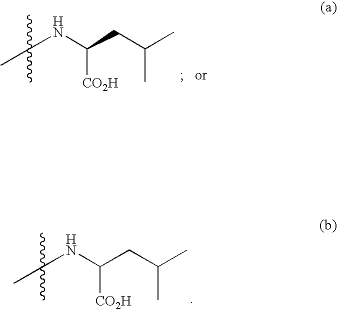

For the purposes of the present invention, an “alkyl” group is defined as a monovalent saturated hydrocarbon which, unless otherwise defined, may be straight-chained or branched, or be or include one or more cyclic groups. An alkyl group may optionally be substituted. An alkyl group may optionally include 1, 2 or 3 heteroatoms N, O or S in its carbon skeleton. Examples of alkyl groups are methyl, ethyl, n-propyl, i-propyl, n-butyl, i-butyl, t-butyl and n-pentyl groups. In one embodiment, an alkyl group is unsubstituted. In another embodiment, an alkyl group is straight-chained or branched. In yet another embodiment, an alkyl group does not include any heteroatoms in its carbon skeleton.

An “alkenyl” group is defined as a monovalent hydrocarbon, which comprises at least one carbon-carbon double bond, and which, unless otherwise defined, may be straight-chained or branched, or be or include one or more cyclic groups. An alkenyl group may optionally be substituted. An alkenyl group may optionally include 1, 2 or 3 heteroatoms N, O or S in its carbon skeleton. Examples of alkenyl groups are vinyl, allyl, but-1-enyl and but-2-enyl groups. In one embodiment, an alkenyl group is unsubstituted. In another embodiment, an alkenyl group is straight-chained or branched. In yet another embodiment, an alkenyl group does not include any heteroatoms in its carbon skeleton.

An “alkynyl” group is defined as a monovalent hydrocarbon, which comprises at least one carbon-carbon triple bond, and which, unless otherwise defined, may be straight-chained or branched, or be or include one or more cyclic groups. An alkynyl group may optionally be substituted. An alkynyl group may optionally include 1, 2 or 3 heteroatoms N, O or S in its carbon skeleton. Examples of alkynyl groups are ethynyl, propargyl, but-1-ynyl and but-2-ynyl groups. In one embodiment, an alkynyl group is unsubstituted. In another embodiment, an alkynyl group is straight-chained or branched. In yet another embodiment, an alkynyl group does not include any heteroatoms in its carbon skeleton.

For the purposes of this invention, an optionally substituted alkyl, alkenyl or alkynyl group may be substituted with one or more of —F, —Cl, —Br, —I, —CF₃, —CCl₃, —CBr₃, —CI₃, —OH, —SH, —NH₂, —CN, —NO₂, —COOH, —OR^{b}, —SR^{b}, —N(R^{b})₂, —CO—R^{b}, —CO—OR^{b}, —CO—N(R^{b})₂, or —R^{b}. —R^{b} is independently hydrogen, or an unsubstituted alkyl group containing 1, 2 or 3 carbon atoms. Preferred substituents are —OH, —NH₂, —NHMe, —NHEt, and —CO₂H.

Optional substituent(s) are not taken into account when calculating the total number of carbon atoms in the parent group substituted with the optional substituent(s). Preferably a substituted group comprises 1, 2 or 3 substituents, or 1 or 2 substituents, or I substituent.

Any optional substituent may be protected. Suitable protecting groups for protecting optional substituents are known in the art, for example from “Protective Groups in Organic Synthesis” by T. W. Greene and P. G. M. Wuts (Wiley-Interscience, 2^{nd} edition, 1991).

Preferably, the compound of the first aspect of the present invention has the formula IA:

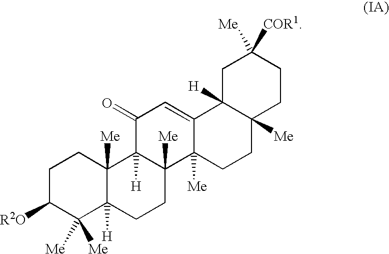

R¹ can be —OR^{a} or —N(R^{a})₂. Preferably, R¹ is —OH or OMe. Alternatively, R¹ may be —OR^{a}. R¹ may be —OR^{a}, with R^{a} being an unsubstituted, straight-chained or branched alkyl, alkenyl or alkynyl group which contains 1, 2, 3, 4, 5 or 6 carbon atoms. Alternatively, R¹ may be —OR^{a}, with R^{a} being a substituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton.

R^{a} can be hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton. If R^{a} is substituted, it is preferably substituted with —OH, —NH, —NHMe, —NHEt, or —CO₂H.

R¹ can be —OR^{a} or —N(R^{a})₂. In one embodiment, R¹ is —NH₂, —NHMe, —NMe₂, —NHEt or —NEt₂; preferably —NMe₂.

R² can be independently each one of

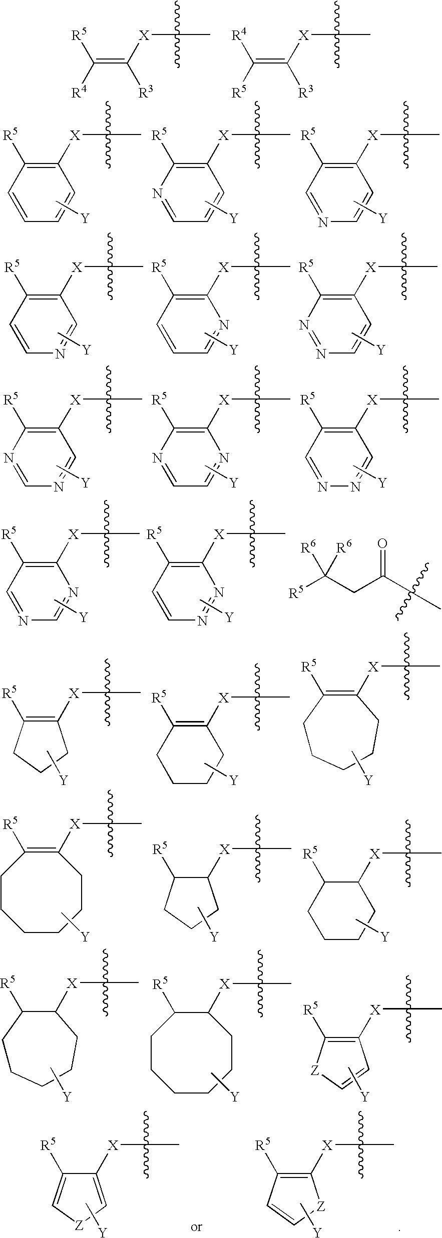

Thus, R² can be

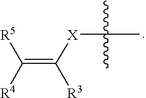

### R² can be

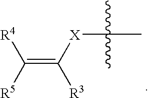

### R² can be

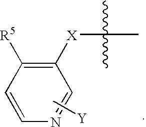

### R² can be

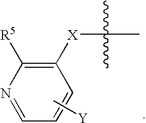

### R² can be

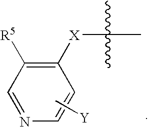

### R² can be

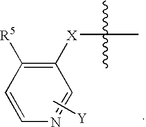

### R² can be

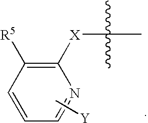

### R² can be

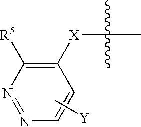

### R² can be

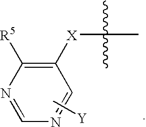

### R² can be

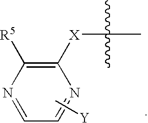

### R² can be

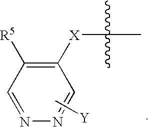

### R² can be

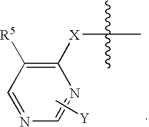

### R² can be

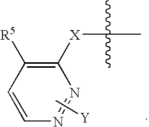

### R² can be

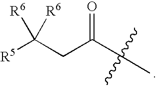

### R² can be

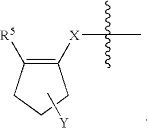

### R² can be

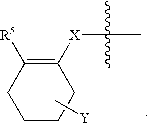

### R² can be

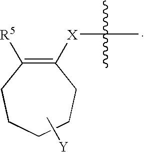

### R² can be

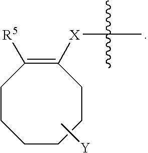

### R² can be

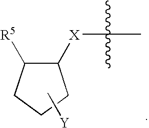

### R² can be

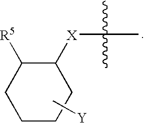

### R² can be

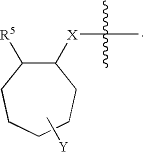

### R² can be

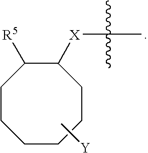

### R² can be

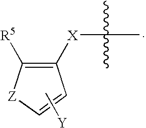

### R² can be

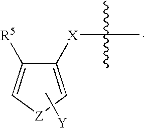

### R² can be

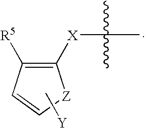

### Preferably, R² is

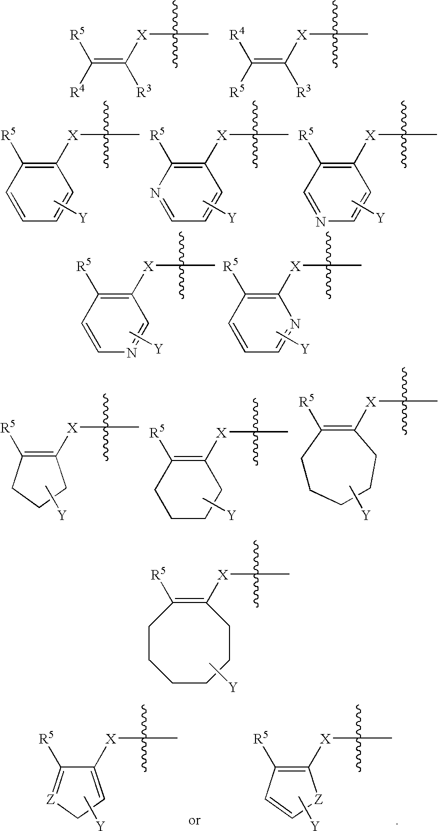

### Preferably, R² is

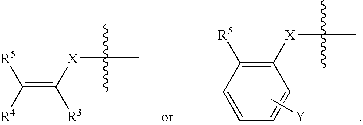

### When R² is

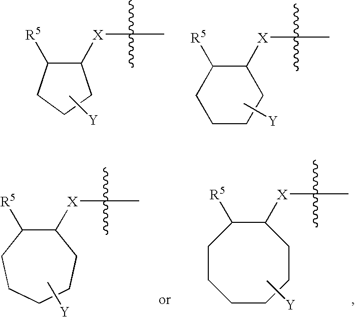

the compound can exist in a cis or a trans form:

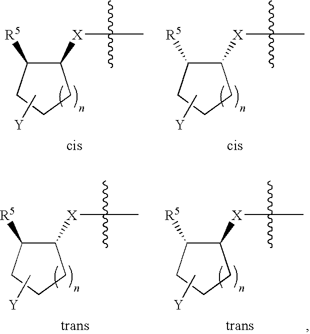

wherein n is 1, 2, 3 or 4.

Preferably, R² is

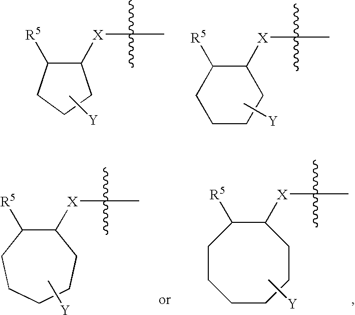

and the compound is one cis-enantiomer, one trans-enantiomer, a mixture of two cis-enantiomers, a mixture of two trans-enantiomers, or a mixture of two cis- and two trans-enantiomers. Preferably, the compound is one cis-enantiomer, or a mixture of two cis-enantiomers. The compound may be the 1S,2S-enantiomer, or the 1S,2R-enantiomer, or 1R,2S-enantiomer, or 1R,2R-enantiomer.

R³ and R⁴ can be independently hydrogen, or an unsubstituted, straight-chained or branched alkyl group which contains 1, 2, 3 or 4 carbon atoms. Preferably, R³ and/or R⁴ is —H or -Me.

R⁵ can be —OH, —CO₂H, —CO₂R⁶, —SO₃H, or —PO₃H₂. Preferably, R⁵ is —OH, —CO₂H or —CO₂R⁶.

It will be appreciated by those skilled in the art that because all R² groups comprise the sub-structure

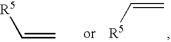

when R⁵ is —OH, the compounds of the present invention can exist in the following tautomeric forms:

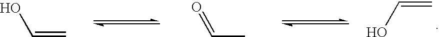

The present invention includes all of these tautomeric forms. An example of a compound having tautomeric forms and falling within the scope of the present invention is compound YP016, the synthesis and biological activity of which is discussed below.

X can be —CO— or —CH₂—. Preferably, X is —CO—.

Y can be —H, —F, —Cl, —Br, —I, -Me, or —OMe. Preferably, Y is hydrogen or fluorine.

In one embodiment, the compound of the present invention is a compound having the formula:

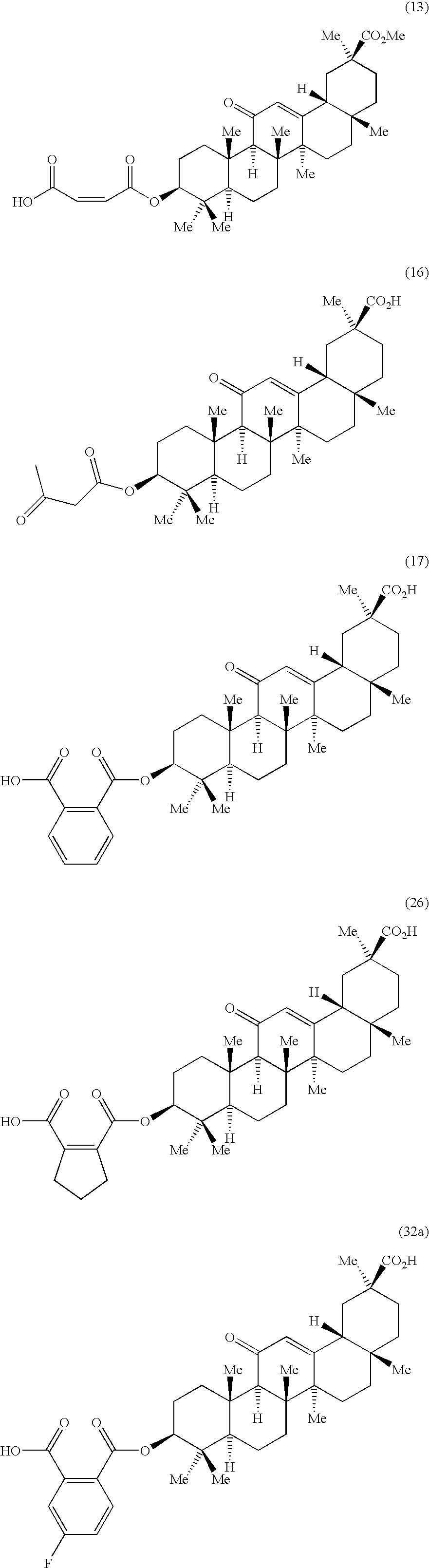

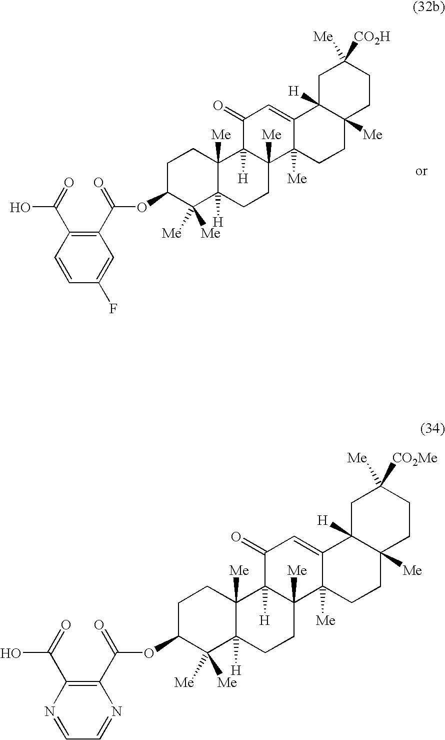

A second aspect of this invention provides a pharmaceutical composition comprising a compound of the present invention, and a pharmaceutically acceptable excipient, carrier or diluent.

The compounds and pharmaceutical compositions of the present invention can be used in medicine. Preferably the patient to be treated is a mammal, preferably a human.

The compounds and pharmaceutical compositions of the present invention can also be used for lowering the endogenous level or activity of retinoic acid in a cell.

The compounds and pharmaceutical compositions of the present invention can also be used for interfering with the biosynthesis of retinoic acid.

The compounds and pharmaceutical compositions of the present invention can also be used for antagonising a retinol dehydrogenase (RDH). Exemplary retinol dehydrogenases are RoDH1, RoDH2, RoDH3, RoDH4, CRAD1, CRAD2, RDH5, retSDR1 or hRoDH-E2 (official gene symbol DHRS9).

The compounds and pharmaceutical compositions of the present invention can also be used for treating a hyperproliferative disorder or photoageing in a patient. Preferably, the treatment lowers the endogenous level or activity of retinoic acid in a cell of the patient. Preferably, the treatment lowers the endogenous level or activity of retinoic acid in a hyperproliferative cell or a cell suffering from photoageing of said patient. Preferably, the treatment lowers the endogenous level or activity of retinoic acid in the cell to an extent that cell proliferation is reduced or prevented, and/or to an extent that cell differentiation is activated or enhanced. Exemplary hyperproliferative disorders are psoriasis, acne vulgaris, acne rosacea, actinic keratosis, solar keratoses, squamous cell carcinoma in situ, basal cell carcinoma, the ichthyoses, hyperkeratoses, disorders of keratinisation such as Darriers disease, palmoplantar keratodermas, pityriasis rubra pilaris, epidermal naevoid syndromes, erythrokeratoderma variabilis, epidermolytic hyperkeratoses, non-bullous ichthyosiform erythroderma, cutaneous lupus erythematosus, lichen planus, cancer, skin cancer, melanoma and dermatofibroma. Preferred hyperproliferative disorders are psoriasis, acne vulgaris, acne rosacea, actinic keratosis, solar keratoses, squamous cell carcinoma in situ, the ichthyoses, hyperkeratoses, disorders of keratinisation such as Darriers disease, palmoplantat keratodermas, pityriasis rubra pilaris, epidermal naevoid syndromes, erythrokeratoderma variabilis, epidermolytic hyperkeratoses, non-bullous ichthyosiform erythroderma, cutaneous lupus erythematosus, lichen planus, or cancer. Preferably, the treatment interferes with the biosynthesis of retinoic acid. Preferably, the treatment antagonises a retinol dehydrogenase (RDH). Exemplary retinol dehydrogenases are RoDH1, RoDH2, RoDH3, RoDH4, CRAD1, CRAD2, RDH5, retSDR1 or hRoDH-E2 (official gene symbol DHRS9).

The compounds and pharmaceutical compositions of the present invention can also be used for reducing or preventing proliferation of a cell in a patient. Preferably, this use of the compounds and pharmaceutical compositions of the present invention lowers the endogenous level or activity of retinoic acid in the cell. Preferably, the cell is a hyperproliferative cell. Preferably, this use results in cell differentiation. Preferably, this use interferes with the biosynthesis of retinoic acid. Preferably, this use antagonises a retinol dehydrogenase (RDH). Exemplary retinol dehydrogenases are RoDH1, RoDH2, RoDH3, RoDH4, CRAD1, CRAD2, RDH5, retSDR1 or hRoDH-E2 (official gene symbol DHRS9).

The compounds and pharmaceutical compositions of the present invention can also be used for activating or enhancing a differentiation program in a cell in a patient. Preferably, this use of the compounds and pharmaceutical compositions of the present invention lowers the endogenous level or activity of retinoic acid in the cell. Preferably, this use interferes with the biosynthesis of retinoic acid. Preferably, this use antagonises a retinol dehydrogenase (RDH). Exemplary retinol dehydrogenases are RoDH1, RoDH2, RoDH3, RoDH4, CRAD1, CRAD2, RDH5, retSDR1 or hRoDH-E2 (official gene symbol DHRS9).

The compounds and pharmaceutical compositions of the present invention can also be used for treating or alleviating the symptoms of a patient suffering from a disorder, wherein the disorder is a retinoid-sensitive disorder treatable by administration of retinoid, or wherein the disorder is a retinoid-sensitive disorder whose symptoms are alleviatable by administration of retinoid, or wherein the disorder corresponds to a side effect of the administration of pharmacological levels of retinoid. Preferably, the treatment lowers the endogenous level or activity of retinoic acid in a cell of the patient. Preferably, the treatment interferes with the biosynthesis of retinoic acid. Preferably, the treatment antagonises a retinol dehydrogenase (RDH). Exemplary retinol dehydrogenases are RoDH1, RoDH2, RoDH3, RoDH4, CRAD1, CRAD2, RDH5, retSDR1 or hRoDH-E2 (official gene symbol DHRS9).

The compounds and pharmaceutical compositions of the present invention can also be used for treating or alleviating the symptoms of a patient suffering from a disease characterised by ectopic, over- or otherwise abnormal expression of a retinoic acid receptor response element (RARE) responsive gene, a vitamin D response element (VDRE) responsive gene, a thyroid hormone receptor response element responsive gene, or a peroxisome proliferator-activated receptor (PPAR) response element responsive gene. Preferably, the treatment lowers the endogenous level or activity of retinoic acid in a cell of the patient. Preferably, the treatment interferes with the biosynthesis of retinoic acid. Preferably, the treatment antagonises a retinol dehydrogenase (RDH). Exemplary retinol dehydrogenases are RoDH1, RoDH2, RoDH3, RoDH4, CRAD1, CRAD2, RDH5, retSDR1 or hRoDH-E2 (official gene symbol DHRS9).

The compounds and pharmaceutical compositions of the present invention can also be used for treating or alleviating the symptoms of a patient suffering from a disease characterised by an imbalance between proliferation and differentiation. Preferably, the treatment lowers the endogenous level or activity of retinoic acid in a cell of the patient. Preferably, the treatment interferes with the biosynthesis of retinoic acid. Preferably, the treatment antagonises a retinol dehydrogenase (RDH). Exemplary retinol dehydrogenases are RoDH1, RoDH2, RoDH3, RoDH4, CRAD1, CRAD2, RDH5, retSDR1 or hRoDH-E2 (official gene symbol DHRS9).

The compounds and pharmaceutical compositions of the present invention can also be used for treating a patient suffering from a disease, disorder or condition, wherein the disease, disorder or condition is a viral infection, HPV infection, HIV infection, HSV infection, HCV infection, EBV (Epstein-Barr virus) infection, warts, postoperative scarring, hypertrophic or keloid scarring, a disorder of melanogenesis, a disorder of pigmentation, enhanced or compromised epidermal barrier function, a disorder of bone growth, bone fracture, osteoporosis, hyperlipidaemia, hepatotoxicity, cirrhosis, hepatitis infection, cutaneous irritation, alopecia, a disorder of fertility, a disorder of spermatogenesis, a disorder of egg implantation, depression, seasonal affective disorder, atherosclerosis, or a disorder of angiogenesis. A preferred disease, disorder or condition is a viral infection, HPV infection, HIV infection, HSV infection, HCV infection, warts, postoperative scarring, hypertrophic or keloid scarring, a disorder of melanogenesis, a disorder of pigmentation, enhanced or compromised epidermal barrier function, a disorder of bone growth, bone fracture, osteoporosis, hyperlipidaemia, hepatotoxicity, cirrhosis, hepatitis infection, cutaneous irritation, alopecia, a disorder of fertility, a disorder of spermatogenesis, a disorder of egg implantation, depression, seasonal affective disorder, atherosclerosis, or a disorder of angiogenesis. Preferably, the treatment lowers the endogenous level or activity of retinoic acid in a cell of the patient. Preferably, the treatment interferes with the biosynthesis of retinoic acid. Preferably, the treatment antagonises a retinol dehydrogenase (RDH). Exemplary retinol dehydrogenases are RoDH1, RoDH2, RoDH3, RoDH4, CRAD1, CRAD2, RDH5, retSDR1 or hRoDH-E2 (official gene symbol DHRS9).

The compounds and pharmaceutical compositions of the present invention can also be used for the inhibition of an enzyme. The inhibition of the enzyme can occur in vitro or in vivo. Preferably, the enzyme is a retinol dehydrogenase (RDH), such as RoDH1, RoDH2, RoDH3, RoDH4, CRAD1, CRAD2, RDH5, retSDR1 or hRoDH-E2 (official gene symbol DHRS9).

A third aspect of this invention provides a use of a compound of the present invention for the manufacture of a medicament for the treatment of a hyperproliferative disorder or photoageing in a patient. Preferably, the medicament is capable of lowering the endogenous level or activity of retinoic acid in a cell of the patient. Preferably, the medicament is capable of lowering the endogenous level or activity of retinoic acid in a hyperproliferative cell or a cell suffering from photoageing of said patient. Preferably, the medicament is capable of lowering the endogenous level or activity of tetinoic acid in the cell to an extent that cell proliferation is reduced or prevented, and/or to an extent that cell differentiation is activated or enhanced. Exemplary hyperproliferative disorders are psoriasis, acne vulgaris, acne rosacea, actinic keratosis, solar keratoses, squamous cell carcinoma in situ, basal cell carcinoma, the ichthyoses, hyperkeratoses, disorders of keratinisation such as Darriers disease, palmoplantar keratodetmas, pityriasis rubra pilaris, epidermal naevoid syndromes, erythrokeratoderma variabilis, epidermolytic hyperkeratoses, non-bullous ichthyosiform erythroderma, cutaneous lupus erythematosus, lichen planus, cancer, cancer, melanoma and dermatofibroma. The third aspect of this invention also provides a use of a compound of the present invention for the manufacture of a medicament for reducing or preventing proliferation of a cell in a patient. Preferably, the medicament is capable of lowering the endogenous level or activity of retinoic acid in the cell. Preferably, the cell is a hyperproliferative cell. Preferably, the medicament is capable of inducing cell differentiation.

The third aspect of this invention also provides a use of a compound of the present invention for the manufacture of a medicament for activating or enhancing a differentiation program in a cell in a patient. Preferably, the medicament is capable of lowering the endogenous level or activity of retinoic acid in the cell. The third aspect of this invention also provides a use of a compound of the present invention for the manufacture of a medicament for treating or alleviating the symptoms of a patient suffering from a disorder, wherein the disorder is a retinoid-sensitive disorder treatable by administration of retinoid, or wherein the disorder is a retinoid-sensitive disorder whose symptoms are alleviatable by administration of retinoid, or wherein the disorder corresponds to a side effect of the administration of pharmacological levels of retinoid. Preferably, the medicament is capable of lowering the endogenous level or activity of retinoic acid in a cell of the patient.

The third aspect of this invention also provides a use of a compound of the present invention for the manufacture of a medicament for treating or alleviating the symptoms of a patient suffering from a disease characterised by ectopic, over- or otherwise abnormal expression of a retinoic acid receptor response element (RARE) responsive gene, a vitamin D response element (VDRE) responsive gene, a thyroid hormone receptor response element responsive gene, or a peroxisome proliferator-activated receptor (PPAR) response element responsive gene. Preferably, the medicament is capable of lowering the endogenous level or activity of retinoic acid in a cell of the patient.

The third aspect of this invention also provides a use of a compound of the present invention for the manufacture of a medicament for treating or alleviating the symptoms of a patient suffering from a disease characterised by an imbalance between proliferation and differentiation. Preferably, the medicament is capable of lowering the endogenous level or activity of retinoic acid in a cell of the patient.

The third aspect of this invention also provides a use of a compound of the present invention for the manufacture of a medicament for treating a patient suffering from a disease, disorder or condition, wherein the disease, disorder or condition is a viral infection, HPV infection, HIV infection, HSV infection, HCV infection, EBV infection, warts, postoperative scarring, hypertrophic or keloid scarring, a disorder of melanogenesis, a disorder of pigmentation, enhanced or compromised epidermal barrier function, a disorder of bone growth, bone fracture, osteoporosis, hyperlipidaemia, hepatotoxicity, cirrhosis, hepatitis infection, cutaneous irritation, alopecia, a disorder of fertility, a disorder of spermatogenesis, a disorder of egg implantation, depression, seasonal affective disorder, atherosclerosis, or a disorder of angiogenesis. Preferably, the medicament is capable of lowering the endogenous level or activity of retinoic acid in a cell of the patient.

In all of the above described uses of the third aspect of the present invention, preferably, the medicament is capable of interfering with the biosynthesis of retinoic acid. Preferably, the medicament is capable of antagonising a retinol dehydrogenase (RDH). Exemplary retinol dehydrogenases are RoDH1, RoDH2, RoDH3, RoDH4, CRAD1, CRAD2, RDH5, retSDR1 or hRoDH-E2 (official gene symbol DHRS9).

The third aspect of this invention also provides a use of a compound of the present invention for the manufacture of a medicament for the inhibition of an enzyme. The inhibition of the enzyme can occur in vitro or in vivo. Preferably, the enzyme is a retinol dehydrogenase (RDH), such as RoDH1, RoDH2, RoDH3, RoDH4, CRAD1, CRAD2, RDH5, retSDR1 or hRoDH-E2 (official gene symbol DHRS9).

A fourth aspect of this invention provides a method of treating a patient suffering from a hyperproliferative disorder or photoageing, which method comprises administering a compound of the present invention. Preferably the method lowers the endogenous level or activity of retinoic acid in a cell of the patient. Preferably the method lowers the endogenous level or activity of retinoic acid in a hyperproliferative cell or a cell suffering from photoageing of said patient. Preferably the method lowers the endogenous level or activity of retinoic acid in the cell to an extent that cell proliferation is reduced or prevented, and/or to an extent that cell differentiation is activated or enhanced. Exemplary hyperproliferative disorders are psoriasis, acne vulgaris, acne rosacea, actinic keratosis, solar keratoses, squamous cell carcinoma in situ, basal cell carcinoma, the ichthyoses, hyperkeratoses, disorders of keratinisation such as Darriers disease, palmoplantar keratodermas, pityriasis rubra pilaris, epidermal naevoid syndromes, erythrokeratoderma variabilis, epidermolytic hyperkeratoses, non-bullous ichthyosiform erythroderma, cutaneous lupus erythematosus, lichen planus, cancer, skin cancer, melanoma and dermatofibroma.

The fourth aspect of this invention also provides a method of reducing or preventing proliferation of a cell, which method comprises contacting the cell with a compound of the present invention. Preferably the method lowers the endogenous level or activity of retinoic acid in the cell. Preferably the cell is a hyperproliferative cell. Preferably the method results in cell differentiation.

The fourth aspect of this invention also provides a method of activating or enhancing a differentiation program in a cell, which method comprises contacting the cell with a compound of the present invention. Preferably the method lowers the endogenous level or activity of retinoic acid in the cell.

The fourth aspect of this invention also provides a method of treating or alleviating the symptoms of a patient suffering from a disorder, wherein the disorder is a retinoid-sensitive disorder treatable by administration of retinoid, or wherein the disorder is a retinoid-sensitive disorder whose symptoms are alleviatable by administration of retinoid, or wherein the disorder corresponds to a side effect of the administration of pharmacological levels of retinoid, which method comprises administering a compound of the present invention to the patient. Preferably the method lowers the endogenous level or activity of retinoic acid in a cell of the patient.

The fourth aspect of this invention also provides a method of treating or alleviating the symptoms of a patient suffering from a disease characterised by ectopic, over- or otherwise abnormal expression of a retinoic acid receptor response element (RARE) responsive gene, a vitamin D response element (VDRE) responsive gene, a thyroid hormone receptor response element responsive gene, or a peroxisome proliferator-activated receptor (PPAR) response element responsive gene, which method comprises administering a compound of the present invention to the patient. Preferably the method lowers the endogenous level or activity of retinoic acid in a cell of the patient.

The fourth aspect of this invention also provides a method of treating or alleviating the symptoms of a patient suffering from a disease characterised by an imbalance between proliferation and differentiation, which method comprises administering a compound of the present invention to the patient. Preferably the method lowers the endogenous level or activity of retinoic acid in a cell of the patient.

The fourth aspect of this invention also provides a method of treating a patient suffering from a disease, disorder or condition, which method comprises administering a compound of the present invention to the patient, wherein the disease, disorder or condition is a viral infection, HPV infection, HIV infection, HSV infection, HCV infection, EBV infection, watts, postoperative scarring, hypertrophic or keloid scarring, a disorder of melanogenesis, a disorder of pigmentation, enhanced or compromised epidermal barrier function, a disorder of bone growth, bone fracture, osteoporosis, hyperlipidaemia, hepatotoxicity, cirrhosis, hepatitis infection, cutaneous irritation, alopecia, a disorder of fertility, a disorder of spermatogenesis, a disorder of egg implantation, depression, seasonal affective disorder, atherosclerosis, or a disorder of angiogenesis. Preferably the method lowers the endogenous level or activity of retinoic acid in a cell of the patient.

In all of the above described methods of the fourth aspect of the present invention, preferably, the method comprises interfering with the biosynthesis of retinoic acid. Preferably, the method comprises antagonising a retinol dehydrogenase (RDH). Exemplary retinol dehydrogenases are RoDH1, RoDH2, RoDH3, RoDH4, CRAD1, CRAD2, RDH5, retSDR1 or hRoDH-E2 (official gene symbol DHRS9).

The fourth aspect of this invention also provides a method of inhibiting an enzyme, comprising administering an inhibitory amount of a compound or a pharmaceutical composition of the present invention to a patient in need thereof. The inhibition of the enzyme can occur in vitro, ex vivo or in vivo. Preferably, the enzyme is a retinol dehydrogenase (RDH), such as RoDH1, RoDH2, RoDH3, RoDH4, CRAD1, CRAD2, RDH5, retSDR1 or hRoDH-E2 (official gene symbol DHRS9).

A fifth aspect of this invention provides a method of synthesising a compound of the present invention, using 18β-glycyrrhetinic acid as a starting material. Preferably, the 3-hydroxyl group of 18β-glycyrrhetinic acid is esterified or alkylated. Preferably, the 20-carboxyl group of 18β-glycyrrhetinic acid is esterified or converted into an amide group.

### BRIEF DESCRIPTION OF THE DRAWINGS

The present invention will now be described by way of example with reference to the accompanying drawings in which:

FIGS. 1a and 1b are graphs showing the effects of carbenoxolone sodium on NHEKa proliferation for comparison.

FIGS. 2a to 2d are graphs showing the effects of a compound of the present invention (YP013) on NHEKa proliferation.

FIGS. 3a to 3d are graphs showing the effects of a compound of the present invention (YP015) on NHEKa proliferation.

FIGS. 4a to 4d are graphs showing the effects of a compound of the present invention (YP016) on NHEKa proliferation.

FIGS. 5a to 5d are graphs showing the effects of a compound of the present invention (YP017) on NHEKa proliferation.

FIGS. 6a to 6d are graphs showing the effects of carbenoxolone and ten compounds of the present invention (YP013, YP015, YP016, YP017, YP022, YP023, YP024, YP026, YP032 and YP034) on NHEKa proliferation.

FIGS. 7a to 7g are graphs showing the effects of carbenoxolone and six compounds of the present invention (YP013, YP015, YP016, YP023, YP024 and YP026) on the viability of NHEKa.

FIGS. 8a to 8h are graphs illustrating the effect of eight compounds of the present invention (YP013, YP015, YP016, YP017, YP022, YP024, YP026 and YP032) compared to carbenoxolone, at increasing concentrations of the inhibitors, on the rate of hRoDH-E2 activity, assayed in E. coli RIL (+hRoDH-E2) SS, and determined by monitoring the generation of retinal at 400 nm. Retinal generation (mAbs/min) is expressed as a percentage of the control reaction (2.5% DMSO, no inhibitor). The inhibitors were dissolved in DMSO (2.5% final concentration) and included at concentrations in the range of 15.625-250 μM. The effect of carbenoxolone is included as a reference. The assay was repeated three times independently on a single stock of SS, and the SEM is indicated by error bars.

FIG. 9 is a graph summarising the effect of carbenoxolone and eight compounds of the present invention (YP013, YP015, YP016, YP017, YP022, YP024, YP026 and YP032), used at a concentration of 15.625 μM, on hRoDH-E2 activity.

In the figures, “Cont.” stands for control and “CBX” stands for carbenoxolone.

### DETAILED DESCRIPTION OF THE INVENTION

Without wishing to be bound by theory, it is currently believed that the compounds, compositions and methods of the present invention work by reducing the level or activity of endogenous retinoic acid, typically to about or below a physiological concentration of that species. Preferably, the level of endogenous retinoic acid is reduced by 10%, 20%, 30%, 40%, 50%, 60%, 70%, 80% or more by the compounds, compositions and methods of the present invention.

According to the present invention, the concentration of retinoic acid is varied about the switch point in order to effect the therapy or effects described here. Most preferably, the level of endogenous retinoic acid is reduced to about or below the switch point. The “switch point” is the concentration of retinoic acid in a cell about which a change in concentration of retinoic acid in either direction (i.e. increase or reduction) will change the differentiative or proliferative fate of the cell. Concentrations of retinoic acid above the switch point cause the cell to undergo proliferation, while concentrations of retinoic acid below the switch point cause the cell to undergo differentiation. The switch point may therefore be determined in any cell or cell type or disease state according to this criterion, by methods known in the art.

In a normal undiseased cell, the switch point is typically at or about the physiological retinoic acid concentration. The physiological retinoic acid concentration in a normal cell is between about 4×10⁻⁹ molar and 1×10⁻⁸ molar (i.e. between about 4 to 10 nanomolar), and accordingly, this range may be taken as a working range for the switch point.

In a preferred embodiment of the present invention, the endogenous level of retinoic acid is reduced by 10%, 20%, 30%, 40%, 50%, 60%, 70%, 80% or more below the switch point. Thus, in one embodiment, the compounds, compositions and methods of the present invention rely on reducing the endogenous retinoic acid level in a relevant cell, for example, a diseased cell or a cell in a diseased individual, to below about 10, 5 or 1 nanomolar, or even below about 750, 500, 50 or 1 picomolar. Preferably, the concentration of endogenous retinoic acid is reduced below the switch point, but is maintained at a high enough level to avoid cell death.

Endogenous or intracellular retinoic acid levels may be assayed by various means as known in the art.

Although the primary benefit of the compounds, compositions and methods of the present invention is the reduction of cellular proliferation, treating the hyperproliferative cells or photoaged cells may induce differentiation as an additional benefit.

The compounds, compositions and methods of the present invention are useful for the treatment of a variety of hyperproliferative diseases including psoriasis and cancer. In particular, the compounds, compositions and methods of the present invention are especially useful for the treatment of psoriasis. The present invention is also useful in treating or alleviating the symptoms of photoageing or photodamage. It will be appreciated that cells of patients suffering from skin hyperproliferative diseases (as described in further detail below), photoaged or photodamaged cells, as well as cancer cells may share many properties with each other. For example, cells of a patient exposed to ultraviolet radiation may display symptoms of photoageing; in addition, these cells may develop into various carcinomas such as basal cell carcinoma or squamous cell carcinoma as a result of such exposure.

In a particular embodiment, the compounds, compositions and methods of the present invention are suitable for treating a hyperproliferative disease, in particular a hyperproliferative disease which affects the skin. Photoageing, neoplasms and cancer are also suitably treated, and other diseases and conditions disclosed below.

The compounds, compositions and methods of the present invention may also be used to treat or alleviate the symptoms of a patient suffering from any disease in which there is an imbalance between proliferation and differentiation. Thus, any condition, in which there is a failure in the normal controls, which regulate the differentiative or proliferative fate of a cell, may be treated. Such a disease will typically involve a cell or tissue type proliferating which normally (i.e. depending on the developmental stage or tissue type) does not or should not proliferate, or which fails to differentiate when the corresponding normal cell or tissue type is in a differentiative state.

In a particular embodiment, the compounds, compositions and methods of the present invention result in a reduction of proliferation, preferably proliferation in vivo, of the hyperproliferative cells. More preferably, proliferation of a population of cells is reduced to 90%, 80% 70%, 60%, 50%, 40%, 30%, 20% or less compared to a similar population of untreated cells. Most preferably, proliferation is reduced to 0%, i.e. the cells cease dividing completely.

As used here, the term “proliferation” is intended to mean the division of cells resulting in growth of a tissue. Proliferative cells are actively dividing and undergo such cell cycle processes as DNA replication, mitosis, cell division etc. Various methods are known by which proliferation may be assayed, for example, by radiolabelling with radioactive nucleotide triphosphates, tritiated thymidine, bromodeoxyuridine etc. to detect replicating cells, by visual examination for mitotic cells etc. Proliferation may also be assayed by expression of markers such as Ki-67, or by determining the increase in cell numbers by direct counting of cultured cells under different conditions.

The term “hyperproliferation” is intended to mean increased proliferation compared to expected proliferation for a cell type, given its stage of development and function. The term is not intended to include transient increased proliferation of cells within a population, for example, in response to a stimulus, which response is expected. For example, it is known that cells in tissues will exhibit increased proliferation when a tissue is injured and more cells are needed to repair a defect in tissue or to replace dead cells. Thus, “hyperproliferation” is specifically intended to refer to increased proliferation in the context of a diseased or otherwise abnormal state, for example, increased proliferation in the case of cancers and psoriasis.

Lowering of retinoic acid levels within cells triggers a permissive differentiation that allows the cells to achieve their normal cell fate. Therefore, in a preferred embodiment of the invention, the compounds, compositions and methods of the present invention result in cell differentiation occurring within some or all of the population of treated cells. Preferably, 10% or more of a hyperproliferative cell population undergoes differentiation after treatment according to the present invention compared with a population of untreated cells. More preferably, this percentage is 20%, 30%, 40%, 50%, 60%, 70%, 80% or more. Most preferably, 90%, 95% or 100% of the cell population undergoes differentiation.

“Differentiation” refers to the process by which unspecialised cells of tissues become specialised for particular functions. Differentiation of a cell may be assessed in various ways, for example, morphologically, or by assaying expression of protein markers specific for the differentiated cell type as known in the art. For example, K1 and K10 keratin are markers for commitment to terminal differentiation of epidermal keratinocytes, and expression is increased when cellular differentiation occurs. In addition to K1 and K10 keratin, other keratin subtypes may be used as markers for different differentiation stages, for example, K5, K14, K16 and K17. Other non-keratin markers, for example, EGF-receptor and β-1 integrin, may also be used as markers for cellular differentiation.

Retinoid-sensitive disorders can be treated by reducing the endogenous level of retinoic acid. Therefore, the compounds, compositions and methods of the present invention may also be used to treat retinoid-sensitive disorders. Examples of retinoid-sensitive disorders are known in the art and include psoriasis, acne, photoageing, cancer, acute promyelocytic leukaemia, psoriasis, disorders of keratinisation e.g. the ichthyoses and keratodermas, scleroderma, vitiligo, eczema, acne vulgaris and acne rosacea, lichen planus, cutaneous lupus erythematosus, premalignant conditions e.g. melanocytic naevus, myelodysplastic syndrome, among others.

Modulating endogenous retinoic acid levels in a cell causes a shift in the proliferative/differentiative fate of the cell. Specifically, lowering the endogenous retinoic acid level causes a cell to cease proliferating and/or start differentiating. Thus, the compounds, compositions and methods of the present invention are generally suitable for treating or alleviating the symptoms of a patient suffering from any disease characterised by an imbalance between proliferation and differentiation, including cancer, tumours, and other skin hyperproliferative diseases as detailed in this document. An imbalance between proliferation and differentiation refers to an increase in the proportion of cells in a tissue engaged in mitosis over that which is normal for that tissue, or a decrease in the proportion of cells in a tissue engaged in mitosis below that which is normal for that tissue.

Compounds, compositions and methods according to a preferred embodiment of the present invention rely on reducing the endogenous or intracellular concentration of retinoic acid in a hyperproliferative cell to below the switch point of a particular cell, in order to cause it to reduce or stop proliferation, and optionally to undergo differentiation. Preferably, the endogenous levels of retinoic acid are lowered to only such an extent as to reduce or stop proliferation, i.e. just below the switch point. However, as indicated above, the retinoic acid concentration within a cell may be reduced further (i.e. to below the switch point, for example, complete deprivation of retinoic acid) to achieve these aims.

It will be appreciated that the switch point is not in proximity to concentrations of retinoic acid that cause toxicity. Therefore, the methods according to the present invention are advantageous in that they would not be expected to have the adverse side effects of cytotoxicity and cell death.

### Skin Hyperproliferative Diseases

Skin hyperproliferation diseases which may be treated by using the compounds, compositions and methods of the present invention include psoriasis, acne vulgaris, acne rosacea, actinic keratosis (solar keratoses—squamous carcinoma in situ), the ichthyoses, hyperkeratoses, disorders of keratinisation such as Darriers disease, palmoplantar keratodermas, pityriasis rubra pilaris, epidermal naevoid syndromes, erythrokeratoderma variabilis, epidermolytic hyperkeratoses, non-bullous ichthyosiform erythroderma, cutaneous lupus erythematosus and lichen planus.

According to the present invention, a patient exhibiting any of the symptoms associated with a skin hyperproliferative disease, for example, a disease as listed above, may be treated with a compound of the present invention to reduce the activity of retinol dehydrogenase and thereby the endogenous retinoic acid levels within hyperproliferative cells in the diseased patient. Such treatment leads to reduction of proliferation of the diseased cells. The compound of the present invention may be applied to a patient on its own or in the form of a pharmaceutical composition as described in more detail below. The effect of treatment of a host with skin proliferation disease may be evaluated by objective criteria such as an improvement of desquamation and erythema, reduction of the size of lesions as well as subjective criteria such as cessation of itching.

In particular, the compounds, compositions and methods of the present invention are suitable for the treatment or alleviation of symptoms of psoriasis. Psoriasis manifests itself as inflamed swollen skin lesions covered with silvery white scale. Characteristics of psoriasis include pus-like blisters (pustular psoriasis), severe sloughing of the skin (erythrodermic psoriasis), drop-like dots (guttate psoriasis) and smooth inflamed lesions (inverse psoriasis).

The causes of psoriasis are currently unknown, although it has been established as an autoimmune skin disorder with a genetic component. One in three people report a family history of psoriasis, but there is no pattern of inheritance. However, there are many cases in which children with no apparent family history of the disease will develop psoriasis. Whether a person actually develops psoriasis may depend on “trigger factors” which include systemic infections such as strep throat, injury to the skin (the Koebner phenomenon), vaccinations, certain medications, and intramuscular injections or oral steroid medications. Once something triggers a person's genetic tendency to develop psoriasis, it is thought that in turn, the immune system triggers the excessive skin cell reproduction.

Skin cells are programmed to follow two possible programs: normal growth or wound healing. In a normal growth pattern, skin cells are created in the basal cell layer, and then move up through the epidermis to the stratum corneum, the outermost layer of the skin. This normal process takes about 28 days from cell birth to death. When skin is wounded, a wound healing program (regenerative maturation) is triggered, in which cells are produced at a much faster rate, the blood supply increases and localized inflammation occurs. Lesional psoriasis is characterised by cell growth in the alternate growth program. Skin cells (keratinocytes) switch from the normal growth program to regenerative maturation, cells are created and pushed to the surface in as little as 2 to 4 days, and the skin cannot shed the cells fast enough. The excessive skin cells build up and form elevated, scaly lesions. The white scale (“plaque”) that usually covers the lesion is composed of dead skin cells, and the redness of the lesion is caused by increased blood supply to the area of rapidly dividing skin cells.

Psoriasis is a genetically determined disease of the skin characterised by two biological hallmarks. First, there is a profound epidermal hyperproliferation related to accelerated and incomplete differentiation. Second, there is a marked inflammation of both epidermis and dermis with an increased recruitment of T lymphocytes, and in some cases, formation of neutrophil microabscesses. Many pathologic features of psoriasis can be attributed to alterations in the growth and maturation of epidermal keratinocytes, with increased proliferation of epidermal cells, occurring within 0.2 mm of the skin's surface. Traditional investigations into the pathogenesis of psoriasis have focused on the increased proliferation and hyperplasia of the epidermis. In normal skin, the time for a cell to move from the basal layer through the granular layer is 4 to 5 weeks. In psoriatic lesions, the time is decreased sevenfold to tenfold because of a shortened cell cycle time, an increase in the absolute number of cells capable of proliferating, and an increased proportion of cells that are actually dividing. The hyperproliferative phenomenon is also expressed, although to a substantially smaller degree, in the clinically uninvolved skin of psoriatic patients.

A common form of psoriasis, psoriasis vulgaris, is characterised by well-demarcated erythematous plaques covered by thick, silvery scales. A characteristic finding is the isomorphic response (Koebner phenomenon), in which new psoriatic lesions arise at sites of cutaneous trauma.

Lesions are often localized to the extensor surfaces of the extremities, and the nails and scalp are also commonly involved. Much less common forms include guttate psoriasis, a form of the disease that often erupts following streptococcal pharyngitis, and pustular psoriasis, which is characterised by numerous sterile pustules, often 2 to 5 mm in diameter, on the palms and soles or distributed over the body.

The compounds, compositions and methods of the present invention are also suitable for the treatment of acne. Acne affects large patient populations and is a common inflammatory skin disorder which usually localizes on the face. Fortunately, the disease usually disappears and in the interval of months or years between onset and resolution, therapy, although not curative, can satisfactorily suppress the disease in the majority of patients.

A small number of acne patients with severe disease show little or no response to intensive therapeutic efforts including the use of high doses of oral tetracycline, dapsone, prednisone, and, in women, estrogen. In many cases, these drugs afford only a modest degree of control while the side effects of these agents severely restrict their usefulness. Patients with nodulocystic acne suffer from large, inflammatory, suppurative nodules appearing on the face, and frequently the back and chest. In addition to their appearance, the lesions are tender and often purulently exudative and hemorrhagic. Disfiguring scars are frequently inevitable.

Therapies for acne involve local and systemic administration of retinoids. Topical application of all-trans-retinoic acid (tretinoin) has been tried with some success, particularly against comedones or blackheads, but this condition frequently returns when the treatment is withdrawn.

Objective methods which are employed for establishing the effect of treatment of psoriasis patients include the resolution of plaques by visual monitoring and with photography. The visual scoring is done using PASI (Psoriasis Area and Severity Index) score (see A. J. Fredericksson, B. C. Peterssonn, Dermatologies, 1978, 157, pp 238-244).

### Neoplasms and Cancer

The compounds, compositions and methods of the present invention may also be used for inhibiting the proliferation and optionally reversing the transformed phenotype of hyperproliferative cancer cells.

Retinoids have been shown experimentally to effect tumour development and growth by several mechanisms including, influencing carcinogen activation, growth factors, angiogenesis, collagenase production and modifying the host immune response (Gollnick Retinoids, 1997, 13, pp 6-12). In vitro retinoic acid has been shown to induce differentiation and/or inhibit clonal expansion of several tumour cell lines including human acute myeloid leukaemia, neuroblastoma, teratocarcinoma, melanoma and rat rhabdomyosarcoma cells. The most responsive cells to retinoic acid induced differentiation are promyelocytic leukaemic cells (Smith et al, Journal of Clinical Oncology, 1992, 10, pp 839-864). The synthetic oral retinoids isotretinoin, etretinate and acitretin and topical isotretinoin and retinoic acid have been shown in controlled clinical trials to induce resolution of premalignant and some malignant non-melanocytic skin cancers (Gollnick, Retinoids, 1997, 13, pp 6-12; Kraemer et al, N Engl J Med, 1988, 318, pp 1633-7). Etretinate and acitretin have been shown to reduce the growth of small basal cell carcinomas (BCC) and prevent new BCC lesions developing in individuals with the Gorlin-Goltz syndrome (Goldberg et al, J Am Acad Dermatol, 1989, 21, pp 144-5). Oral and topical retinoids have been shown to reduce the number/prevent progression of pre-cancerous actinic keratoses and bowenoid keratoses (Meyskens et al, J Am Acad Dermatol, 1986, 15, pp 822-5; Moriarty et al, Lancet, 1982, 1, pp 364-5) and to treat established squamous cell carcinomas (Levine et al, Arch Dermatol, 1989, 125, pp 1225-30). The topical application of TRA (all-trans retinoic acid) has also produced resolution of dysplastic naevi in a half-sided study (Meyskens et al, J Am Acad Dermatol, 1986, 15, pp 822-5).

The most responsive haematological malignancy to retinoid therapy is acute promyelocytic leukaemia (APL) in which retinoids can induce complete remission without a period of bone marrow aplasia (Stone et al, Blood, 1988, 71, pp 690-696; Wallace, Am J Hematol, 1989, 31, pp 266-268). In patients with an excised head and neck squamous cell carcinomas oral retinoids have been shown to reduce the development of second epithelial tumours, 4% compared to 24% in placebo treated group (Smith et al, Journal of Clinical Oncology, 1992, 10, pp 839-864). Regression of advanced cervical squamous cell carcinoma has been induced by a combination of retinoic acid and IFN-alpha (Lippman et al, J Natl Cancer Inst, 1992, 81, pp 241-245). Retinoids have also been shown to be effective in animal models of other solid tumours including breast, prostate and bladder (Smith et al, Journal of Clinical Oncology, 1992, 10, pp 839-864; Whelan, Eur Urol, 1999, 35, 424-428). However existing retinoid therapy is limited by the substantial toxicities that result from activation of multiple signalling pathways (Singh and Lippman, Oncology, 1998, 12, pp 1643-1659).

Certain triterpenoid acid derivatives have been found to exhibit selectin ligand activity and leukotriene biosynthetic inhibitory activity and thus are thought to be useful in the treatment of cancer (U.S. Pat. No. 5,679,644).

Although the most effective use of retinoids has been in the prevention of tumours rather than treatment of established lesions (Bollag and Holdener, Annals of Oncology, 1992, 3, pp 513-526; Kemmett and Hunter, Hospital Update, March 1988, pp 1301-1313; Shi-Yong and Lotan, Drugs of the Future, 1988, 23, pp 621-634), the compounds, compositions and methods of the present invention may be used both for prevention and for treatment.

Thus, any of the above conditions may be treated or alleviated by the compounds, compositions and methods of the present invention. In particular, the compounds, compositions and methods are useful for treating any tumour, carcinoma etc, which has been treated successfully or unsuccessfully with retinoid therapy. The methods and compositions are also useful to treat premalignant conditions i.e. to prevent their progression to actual malignancy. Reduction in endogenous retinoic acid levels inhibits angiogenesis, and therefore such reduction may be used to prevent the spread of tumours. In particular, such reduction in endogenous retinoic acid levels may be achieved by inhibiting or preventing synthesis of retinoic acid by inhibiting retinol dehydrogenase. Specific examples of turnouts include melanocytic naevus and myelodysplastic syndrome.

The present method can be performed on cells in culture, e.g. in vitro or ex vivo, or can be performed on cells present in an animal subject, e.g. as part of an in vivo therapeutic protocol. The therapeutic regimen can be carried out on a human or other animal subject. The terms “anti-neoplastic agent” and “anti-proliferative agent” are used interchangeably herein and includes agents that have the functional property of inhibiting the proliferation of a hyperproliferative cell, e.g. inhibit the development or progression of a neoplasm.

Preferably, a therapeutically effective anti-neoplastic amount of a compound of the present invention is used, i.e. an amount which is effective, upon single or multiple dose administration to the patient, in inhibiting the growth of neoplastic cells or in prolonging the survivability of the patient with such neoplastic cells beyond that expected in the absence of such treatment. As used herein, “inhibiting the growth” of the neoplasm includes the slowing, interrupting, arresting or stopping of its growth and metastases and does not necessarily indicate a total elimination of the neoplastic growth. The compound of the present invention may also be used as a prophylactic, in a prophylactically effective anti-neoplastic amount, i.e. an amount which is effective, upon single or multiple dose administration to the patient, in preventing or delaying the occurrence of the onset of a neoplastic disease state. Particular cancers which are treatable with the methods and compositions of the present invention include basal cell carcinoma, squamous cell carcinoma, dysplastic naevi, and malignant melanoma. It will be appreciated that photoaged cells, as well as many skin proliferative diseases, may exhibit many of the properties of premalignant cancer cells, and the present invention may also usefully be employed in the treatment of such cells.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumours”, which may be either benign, premalignant or malignant.

The compounds of the present invention may be tested initially in vitro for their inhibitory effects on the proliferation of neoplastic cells. Examples of cell lines that can be used are transformed cells, e.g. the human promyeloid leukaemia cell line HL-60, and the human myeloid leukaemia U-937 cell line (Abe E. et al, Proc. Natl. Acad. Sci. USA, 1981, 78, pp 4990-4994; L. N. Song and T. Cheng, Biochem Pharmacol, 1992, 43, pp 2292-2295; J. Y. Zhou et al, Blood, 1989, 74, pp 82-93; U.S. Pat. No. 5,401,733; U.S. Pat. No. 5,087,619). Alternatively, the anti-tumoural effects of such an agent can be tested in vivo using various animal models known in the art and summarised in R. Bouillon et al, Endocrine Reviews, 1995, 16(2), p 233 (Table E), which is incorporated by reference herein. For example, SL mice are routinely used in the art as models for MI myeloid leukaemia (Honma et al, Cell Biol, 1983, 80, pp 201-204; T. Kasukabe et al, Cancer Res, 1987, 47, pp 567-572); breast cancer studies can be performed in, for example, nude mice models for human MX1 (ER) U. Abe et al, Endocrinology, 1991, 129, pp 832-837); other cancers, e.g. colon cancer, melanoma osteosarcoma, can be characterised in, for example, nude mice models (J. A. Eisman et al, Cancer Res, 1987, 47, pp 21-25; A. Kawaura et al, Cancer Lett, 1990, 55, pp 149-152; A. Belleli, Carcinogenesis, 1992, 13, pp 2293-2298; H. Tsuchiya et al, J Orthopaed Res, 1993, 11, pp 122-130).

In certain embodiments, the compounds of the present invention can be used in combinatorial therapy with conventional cancer chemotherapeutics. Conventional treatment regimens for tumours include radiation, drugs, or a combination of both. In addition to radiation, the following drugs, usually in combinations with each other, are often used to treat acute tumours: vincristine, prednisone, methotrexate, mercaptopurine, cyclophosphamide and cytarabine. In chronic leukaemia, for example, busulfan, melphalan and chlorambucil can be used in combination. All of the conventional anti-cancer drugs are highly toxic and tend to make patients quite ill while undergoing treatment. Vigorous therapy is based on the premise that unless every cancerous cell is destroyed, the residual cells will multiply and cause a relapse.

The method of the present invention can also be useful in treating malignancies of the various organ systems, such as affecting lung, breast, lymphoid, gastrointestinal and genitourinary tract as well as adenocarcinomas which include malignancies such as most colon cancers, renal cell carcinoma, prostate cancer and/or testicular tumours, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g. which include malignant turnouts composed of carcinomatous and sarcomatous tissues. An “adenocarcinoma” refers to a carcinoma derived from glandular tissue or in which the turnout cells form recognizable glandular structures.

The term “sarcoma” is art recognized and refers to malignant tumours of mesenchymal derivation.

According to the general paradigm that reducing endogenous retinoic acid levels by inhibiting retinol dehydrogenase leads to reduction of proliferation of transformed cells, exemplary solid turnouts that can be treated according to the method of the present invention include sarcomas and carcinomas such as, but not limited to, fibrosatcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumour, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumour, cervical cancer, testicular tumour, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Determination of a therapeutically effective anti-neoplastic amount or a prophylactically effective anti-neoplastic amount of the compound of the present invention can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician, the severity of the condition being treated and the particular compound being employed. In determining the therapeutically effective anti-neoplastic amount or dose, and the prophylactically effective anti-neoplastic amount or dose, a number of factors are considered by the attending clinician, including, but not limited to, the specific hyperplastic/neoplastic cell involved; the particular compound administered; pharmacodynamic characteristics of the particular compound and its mode and route of administration; the desired time course of treatment; the species of mammal; its size, age and general health; the specific disease involved; the degree, involvement or severity of the disease; the response of the individual patient; the bioavailability characteristics of the preparation administered; the dose regimen selected; the kind of concurrent treatment (i.e. the interaction of the compounds used with other co-administered therapeutics); and other relevant circumstances. U.S. Pat. No. 5,427,916, for example, describes a method for predicting the effectiveness of anti-neoplastic therapy in individual patients, and illustrates certain methods which can be used in conjunction with the treatment protocols of the present invention.

Treatment can be initiated with smaller dosages which are less than the optimum dose of the compound. Thereafter, the dosage should be increased by small increments until the optimum effect under the circumstances is reached. For convenience, the total daily dosage may be divided and administered in portions during the day if desired. Compounds which are determined to be effective for the prevention or treatment of tumours in animals, e.g. dogs and rodents, may also be useful in treatment of tumours in humans. Those skilled in the art of treating turnouts in humans will know, based upon the data obtained in animal studies, the dosage and route of administration of the compounds to humans. In general, the dosage and route of administration in humans is expected to be similar to that in animals. Further considerations relating to dosage are discussed below.

The identification of those patients who are in need of prophylactic treatment for hyperplastic/neoplastic disease states is well within the ability and knowledge of one skilled in the art. Certain of the methods for identification of patients which are at risk of developing neoplastic disease states which can be treated by the compounds, compositions and methods of the present invention, are appreciated in the medical arts, such as family history of the development of a particular disease state and the presence of risk factors associated with the development of that disease state in the subject patient. The present invention also includes other prognostic tests which can be used to make, or to augment a clinical predication about the use of the compounds, compositions and methods of the present invention. A clinician skilled in the art can readily identify such candidate patients, by the use of, for example, clinical tests, physical examination and medical/family history.

### Photoageing

The alterations in the structural and functional components of the skin as a result of prolonged exposure to ultraviolet radiation are collectively referred to as photodamage, dermatoheliosis or photoageing.

The smooth elastic properties of normal skin are maintained by the water retaining barrier provided by the epidermis (Cork, J. Dermatol. Treat, 1997, 8, pp S7-S13) and the support by structural fibrillar proteins, collagen and elastin in the dermis. Chronic exposure to ultraviolet radiation results in macroscopic and microscopic changes in the skin, which are termed photoageing (Gilchrest, Br J Dermatol, 1992, 127, Suppl 41, pp 14-20). The clinical features of photoaged or photodamaged skin include fine and course wrinkles, pigmentary changes, age spots (actinic lentigines), laxity, roughness, sallowness, mottled hyperpigmentation, telangiectasia (prominent fine blood vessels) and several benign, premalignant and malignant neoplasms. These can have a significant impact on certain aspects of quality of life (Gupta & Gupta, Journal of Dermatological Treatment, 1996; Griffiths et al, Griffiths, 1992, 7, pp 261-264). Histologically the epidermis is thickened initially, becoming atrophic in the later stages, with keratinocyte atypia and dysplasia. Dermal elastosis and increased melanocyte activity are also observed. Dysplastic and neoplastic changes such as actinic keratoses and basal and squamous cell carcinomas are also extreme features of photodamaged skin. The dermis contains an increased number of elastic fibres that are thickened and degraded in a disorganised mass and decreased collagen. The dermal blood vessels are dilated and tortuous (Fisher et al., Nature, 1996, 379, pp 335-339).

Several double blind clinical trials have demonstrated that 0.05% tretinoin can reduce the clinical and histological features of photoageing (Olsen et al, J. Am. Acad. Dermatol., 1992, 26, pp 215-224; Weinstein et al, Arch Dermatol, 1991, 127, pp 659-65). After application of tretinoin the epidermis thickens and irregularly sized, shaped and stained cells with oddly shaped nuclei give way to healthier looking keratinocytes (Kligman and Graham, J. Dermatol. Treat, 1993, 4, pp 113-117; Kligman and Leyden, Skin Pharmacol, 1993, 6, pp 78-82). These changes are a result of the “pushing” of the keratinocytes into a more normal pattern of differentiation (Marks, 1996). Supporting this hypothesis, retinoids have been shown to produce rapid clearance of premalignant actinic keratoses (Gilchrest, Br J Dermatol, 1992, 127, Suppl 41, pp 14-20; Moriarty et al, Lancet, 1982, 1, pp 364-5).

Although ageing has been thought to be irreversible, studies made during the last decade have shown that some topical compounds and surgical procedures can improve age-related skin damage (Griffiths et al, Archives of Dermatology, 1995, 131, pp 1037-1044; Roger & Fuleihan, Facelift and adjunctive procedures in the treatment of photodamaged skin, in Photodamage, B. A. Gilchrest ed., Blackwell Science, 1995, pp 259-285; Pierard et al, Maturitas, 1996, 23, pp 273-277; Pierard et al, Dermatology, 1997, 194, pp 398-401). Drug treatment consists of sunscreens, retinoids, antioxidants including vitamin C and E and beta-carotene, alpha-hydroxyacids and oestrogen (Humphreys et al, Journal of American Academy of Dermatology, 1996, 34, pp 638-644; Thibault et al, Dermatology Surgery, 1998, 24, pp 573-577; Weiss et al, JAMA, 1998, 259, pp 527-532). A variety of topical prescription and non-prescription agents are widely available for improving photodamaged skin, the efficacy of which is unclear. Topical retinoic acid treatment results in the increased synthesis of collagens in the dermis and effacement of wrinkles (Griffiths et al, New England Journal of Medicine, 1993, 329, pp 530-535; Kligman, J Invest Dermatol, 1987, 88, pp 12s-17s; Kligman et al, Connect Tissue Res, 1984, 12, pp 139-50). Application of retinoic acid also produces a deposition of linear elastic fibres replacing the tortuous elastic fibres produced by UV irradiation (Tsukahara et al, Br J Dermatol, 1999, 140, pp 1048-1053).

The effects of topical and oral retinoids on photoaged skin therefore appear to involve a modification of the differentiation program of keratinocytes and fibroblasts. Currently the use of retinoids in the treatment and prevention of photoageing is limited by their adverse effects, principally teratogenicity and cutaneous irritation. Cultures of fibroblasts have been used to demonstrate that retinoic acid causes an increased production of collagen and elastin in vitro (Tajima et al, Dermatol Sci, 1997, 15, pp 166-7). The effects of retinoic acid on keratinocyte differentiation/proliferation in vitro have been studied by quantifying the production of keratins (Fuchs and Green, Cell, 1981, 25, pp 617-25; Kim et al, Proc. Natl. Acad. Sci., 1984, 81, pp 4280-4284).

The compounds, compositions and methods of the present invention may be used to treat photoageing in a patient or to alleviate its symptoms. Reducing the endogenous level of retinoic acid within cells of a patient suffering from photoageing produces histological and clinical improvement in photodamaged or photoaged skin. These improvements may be achieved by administration of inhibitors retinol dehydrogenase.

Thus, the compounds of the present invention when applied topically to the skin, reverse the condition associated with photodamage so as to moderate and retard the damage to the skin caused by sun exposure. The damage caused sun exposure may include premature aging, elastosis and wrinkling or other symptoms as described earlier. This damage is more pronounced in older patients. By applying the compounds of the present invention topically to the skin in an amount effective to reverse the conditions associated with photodamage, the acceleration of skin repair is accomplished to enhance the skin with a smoother and younger appearance. The compounds of the present invention should be applied to that portion or area of the skin which is affected by photodamage or in which treatment is desired. The use of the compounds of the present invention in accordance with the present invention can provide the effects of anti-aging and anti-wrinkling, as well as enhance the repair of sun damaged skin.

The compounds of the present invention can be applied in accordance with the present invention to human skin in conventional topical compositions, as described elsewhere. These compositions can be utilized to apply the compounds of the present invention to the skin of the body, particularly the face, legs, arms and hands. The preferred method of application of the compounds of the present invention topically to produce the best effects should start where a patient is between 30 and 55 years of age, when elastosis begins to appear and becomes more pronounced.

Thereafter, the composition of the present invention can be continuously applied to patients to reduce the effects and injury associated with sun exposure. Generally, it is preferred to begin the treatment when the patient reaches approximately 30 years of age and to continue the treatment throughout his life, in order that the effects of elastosis be reduced and to prevent any further progression of photodamage.

The compounds of the present invention can be administered in accordance with this invention in any conventional suitable topical preparation, that is, in combination with any suitable conventional carrier useful for topical administration, as described in further detail elsewhere in this document. Therefore, the compounds of the present invention can be administered in accordance with this invention in any suitable topical composition such as a cream, ointment, soap, solution, lotion, emulsion, shampoo, and the like. Generally, for most efficacious results, these topical compositions contain from about 0.01% to about 0.1% by weight of the total composition of a compound of the present invention, with amounts of from about 0.1% to about 0.01% by weight of the composition being especially preferred. If desired, higher concentrations may be utilized depending upon the nature and extent of elastosis.

In formulating these compositions, any conventional non-toxic, dermatologically acceptable base or carrier in which the compound(s) of the present invention is stable can be utilized. The preferred compositions for use in this invention are the conventionally cosmetic compositions which can contain a cosmetically active ingredient which is topically administered to human skin to provide a cosmetic effect. Among the conventional cosmetically active materials which can be utilized in this composition are included sunscreens, penetration enhancers, moisturizers, surfactants, emollient, colorants, conditioners, bacteriocides, astringents, detergents, and the like. The topical compositions of this invention can, if desired, contain suitable sunscreen agents. Any conventional sunscreen agent can be utilized in formulating the compositions containing the compounds of the present invention which can be utilized in accordance with this invention.

These topical compositions can contain any of the conventional excipients and additives commonly used in preparing topical compositions. Among the conventional additives or excipients, which can be utilized in preparing these cosmetic compositions in accordance with this invention are preservatives, thickeners, perfumes and the like. In addition, conventional antioxidants, such as butylated hydroxyanisoles (BHA), ascorbyl palmitate, propyl gallate, citric acid, butylated hydroxy toluene (BHT), ethoxyquin, tocopherol, and the like can be incorporated into these compositions. The topical compositions can contain conventionally acceptable carriers for topical application. The compositions may contain thickening agents, humectants, emulsifying agents and viscosity stabilizers, such as those generally utilized. In addition, the compositions can contain flavouring agents, colorants, and perfume which are conventional in preparing cosmetic compositions. Other components which may be included in the composition are described elsewhere in this document.

The topical compositions containing the compounds of the present invention can be applied to the skin and should preferably be applied once daily to the skin. For obtaining the reversal of the elastosis so as to impart to the skin a smooth and younger appearance, the topical compositions should preferably be applied for a period of 6 months. After that, compositions which contain the compounds of the present invention should be applied continually to maintain the effect of younger and smoother skin. The preparations can be applied according to the need of the patient as determined by the prescribing physician. In any event, the particular regimen for application of the composition to a patient will typically depend on the age, weight and skin condition of the individual.

The UVB irradiated hairless mouse has been found to be a convenient model for actinic elastosis in the skin (Kligman et al, J. Invest. Dermatol., 1982, 78, pp 181). It has been shown by Johnston et al (in J. Invest. Dermatol., 1984, 82, pp 587) that irradiation with low levels of UVB which simulate realistic solar exposure leads to a significant increase in skin elastin as measured by desmosine content. The amount of this amino acid, which is isolated from acid hydrolysis of elastin, is proportional to the elastin present in the skin (Uitto et al, Lab. Invest., 1973, 49, pp 1216). Treatment of irradiated mice with topical retinoic acid has been shown to normalize the histological features of the skin in which the previously elastic dermis has the appearance of unirradiated tissue (Kligman et al, Conn. Tissue Res., 1984, 12, pp 139; U.S. Pat. No. 4,603,146). Therefore, this model can be used to determine the efficacy of compounds in the repair of sun damaged skin.

Assessment of the efficacy of treatment on humans may be made by any method conventionally known and accepted by the medical profession. This may include subjective assessment by a clinician of the symptoms of photoageing, for example. Wrinkling or roughness of the skin may be measured using optical profilometry of silicone casts of areas of interest, for example, the crow's foot region of the facial skin. Clinical measurements of skin, such as the face and wrinkling of the hands, may also be made. In addition, objective measurements of skin thickness may be made by using a pulsed A-scan ultrasound device. These and other methods of assessing photoageing are reviewed in Craven et al, J. Derm. Treatment., 1996, 7, Suppl 2, pp S23-S27.

### Other Indications

The compounds, compositions and methods of the present invention are useful for treating other ailments, conditions and diseases besides skin hyperproliferative diseases, cancer and photoageing.

Other indications which may be treated or alleviated by the compounds, compositions and methods of the present invention include any viral disease. Retinoids are known to have an anti-vital effect, and some cutaneous viral diseases such as human papilloma virus (HPV) induced warts are retinoid response. The HPV genome is known to contain retinoid responsive elements. Accordingly, the compounds, compositions and methods of the present invention may be used to treat any viral disease, including HPV, lentiviral infection, cytomegalo virus, Epstein-Barr virus (BZLF1), adenovirus, human immunodeficiency virus (HIV), herpes simplex virus (HSV), or hepatitis virus (for example, hepatitis B virus or hepatitis C virus) infection.

For example, the compounds, compositions and methods of the present invention can be used for treatment of post-operative scarring, including treatment of hypertrophic and keloid scarring. Furthermore, the compounds, compositions and methods of the present invention may be used for the stimulation of melanogenesis and for the regulation of pigmentation. Furthermore, reduction of endogenous retinoic acid levels may be used to modulate epidermal barrier function.

Furthermore, particular conditions, diseases, etc. which may be treated by the compounds, compositions and methods of the present invention are those which are characterised by being or corresponding to side effects of therapeutic administration of retinoids. Thus, current retinoid therapy involves administration of pharmacological concentrations of retinoids, orally or topically, to a patient, leading to a number of unwanted side effects. These side effects can be manifested as conditions in their own right, independent of retinoid therapy. Lowering endogenous retinoic acid levels as set out in this document may be used to treat or alleviate such conditions.

Thus, it is known that oral retinoids inhibit bone growth. Reducing the endogenous retinoic acid level in the patient may be used to affect bone growth positively. The compounds, compositions and methods of the present invention may therefore be used as a therapy to enhance bone growth in any condition where bone growth is inhibited. Examples of such conditions include fracture repair and treatment of osteoporosis.

Furthermore, administration of retinoids (for example, orally) can cause hyperlipidaemia. Lowering the endogenous retinoic acid level may be used to lower lipid levels in any condition where high levels of lipid exist. Administration of retinoids can also cause hepatotoxicity, and the compounds, compositions and methods of the present invention may be employed as a means to effect hepatic repair, for example, as a result of cirrhosis or hepatitis infection. Reduction in endogenous retinoic acid levels may be employed to treat, prevent or reverse cutaneous irritation; it is known that retinoids are capable of causing cutaneous irritation when administered, for example, topically. The compounds, compositions and methods of the present invention may be used to treat or reverse alopecia, which may arise from a variety of causes; oral retinoids are known to cause alopecia. The reduction of endogenous retinoic acid levels may be used to enhance fertility, or as a fertility treatment, as oral retinoids are known to interfere with spermatogenesis and egg implantation, and therefore reduce fertility.

Furthermore, it has been reported that the administration of oral retinoids (for example, isotretinoin) as a treatment for acne causes depression and suicide. The compounds, compositions and methods of the present invention may therefore be used as a treatment for depression, optionally in combination with other known anti-depressants. For example, seasonal affective disorder may be treated this way. Other conditions such as atherosclerosis may be treated, as well as any condition involving angiogenesis.

### Pharmaceutical Compositions

The present invention also relates to pharmaceutical compositions comprising one or more compounds of the present invention which may reduce intracellular retinoic acid levels. These may act by inhibiting the activity of retinol dehydrogenase.

While it is possible for the compounds of the present invention to be administered alone, it is preferable to formulate the compounds as pharmaceutical formulations. The pharmaceutical compositions of the present invention comprise an effective amount of a compound of the present invention together with one or more pharmaceutically acceptable carriers. An “effective amount” is an amount sufficient to reduce an intracellular retinoic acid level, preferably to enable a cell to cease proliferation and optionally start differentiating, most preferably to alleviate at least one symptom of a skin proliferation disease, a cancer, or photoageing. The effective amount will vary depending upon the particular disease or symptom to be treated or alleviated, as well as other factors including the age and weight of the patient, how advanced the disease state is, the general health of the patient, the severity of the symptoms, and whether the compound of the present invention is being administered alone or in combination with other therapies.

A pharmaceutical composition may include more than one active ingredient. Pharmaceutical compositions may be administered simultaneously or sequentially, for example, in rotation.

Suitable pharmaceutically acceptable carriers are well known in the art and vary with the desired form and mode of administration of the pharmaceutical formulation. For example, they can include diluents or excipients such as fillers, binders, wetting agents, disintegrators, surface-active agents, lubricants and the like. Typically, the carrier is a solid, a liquid or a vaporizable carrier, or a combination thereof. Each carrier should be “acceptable” in the sense of being compatible with the other ingredients in the formulation and not injurious to the patient. The carrier should be biologically acceptable without eliciting an adverse reaction (e.g. immune response) when administered to the host.

The pharmaceutical compositions of the present invention include those suitable for topical and oral administration, with topical formulations being preferred where the tissue affected is primarily the skin or epidermis (for example, psoriasis and other epidermal hyperproliferative diseases, photoageing, skin cancer, etc). The topical formulations include those pharmaceutical forms in which the composition is applied externally by direct contact with the skin surface to be treated. A conventional pharmaceutical form for topical application includes a soak, an ointment, a cream, a lotion, a paste, a gel, a stick, a spray, an aerosol, a bath oil, a solution and the like. Topical therapy is delivered by various vehicles, the choice of vehicle can be important and generally is related to whether an acute or chronic disease is to be treated. As an example, an acute skin proliferation disease generally is treated with aqueous drying preparations, whereas a chronic skin proliferation disease is treated with hydrating preparations. Soaks are the easiest method of drying acute moist eruptions. Lotions (powder in water suspension) and solutions (medications dissolved in a solvent) are ideal for hairy and intertriginous areas. Ointments or water-in-oil emulsions, are the most effective hydrating agents, appropriate for dry scaly eruptions, but are greasy and depending upon the site of the lesion sometimes undesirable. As appropriate, they can be applied in combination with a bandage, particularly when it is desirable to increase penetration of the pharmaceutical composition into a lesion. Creams or oil-in-water emulsions and gels are absorbable and are the most cosmetically acceptable to the patient (Guzzo et al, in Goodman & Gilman's Pharmacological Basis of Therapeutics, 9th ed., 1996, pp 1593-1595). Cream formulations generally include components such as petroleum, lanolin, polyethylene glycols, mineral oil, glycerin, isopropyl palmitate, glyceryl stearate, cetearyl alcohol, tocopheryl acetate, isopropyl myristate, lanolin alcohol, simethicone, carbomer, methylchlorisothiazolinone, methylisothiazolinone, cyclomethicone and hydroxypropyl methylcellulose, as well as mixtures thereof. Other formulations for topical application include shampoos, soaps, shake lotions, and the like, particularly those formulated to leave a residue on the underlying skin, such as the scalp (Arndt et al, Dermatology in General Medicine, 1993, 2, pp 2838).

In general, the concentration of the compound of the present invention in the topical formulation is in an amount of about 0.5 to 50% by weight of the composition, preferably about 1 to 30%, more preferably about 2 to 20%, and most preferably about 5 to 10%. The concentration used can be in the upper portion of the range initially, as treatment continues, the concentration can be lowered or the application of the formulation may be less frequent. Topical applications are often applied twice daily. However, once-daily application of a larger dose or more frequent applications of a smaller dose may be effective. The stratum corneum may act as a reservoir and allow gradual penetration of a drug into the viable skin layers over a prolonged period of time.

In a topical application, a sufficient amount of a compound of the present invention must penetrate the patient's skin in order to obtain a desired pharmacological effect. It is generally understood that the absorption of a drug into the skin is a function of the nature of the drug, the behaviour of the vehicle, and the skin. Three major variables account for differences in the rate of absorption or flux of different topical drugs or the same drug in different vehicles; the concentration of drug in the vehicle, the partition coefficient of the drug between the stratum corneum and the vehicle, and the diffusion coefficient of the drug in the stratum corneum. To be effective for treatment, a drug must cross the stratum corneum which is responsible for the barrier function of the skin. In general, a topical formulation which exerts a high in vitro skin penetration is effective in vivo. Ostrenga et al (R Pharm. Sci., 1971, 60, pp 1175-1179) demonstrated that in vivo efficacy of topically applied steroids is proportional to the steroid penetration rate into dermatomed human skin in vitro.

A skin penetration enhancer which is dermatologically acceptable and compatible with the compound of the present invention can be incorporated into the formulation to increase the penetration of the compound from the skin surface into epidermal keratinocytes. A skin enhancer which increases the absorption of the compound into the skin reduces the amount of compound needed for an effective treatment and provides for a longer lasting effect of the formulation. Skin penetration enhancers are well known in the art. For example, dimethyl sulfoxide (U.S. Pat. No. 3,711,602); oleic acid and 1,2-butanediol surfactant (Cooper, J. Pharm. Sci., 1984, 73, pp 1153-1156); a combination of ethanol and oleic acid or oleyl alcohol (EP 0,267,617); 2-ethyl-1,3-hexanediol (WO 87/03490); decyl methyl sulphoxide and Azone (Hadgraft, Eur. J. Drug Metab. Pharmacokinet, 1996, 21, pp 165-173); alcohols, sulphoxides, fatty acids, esters, Azone, pyrrolidones, urea and polyols (Kalbitz et al, Phatmazie, 1996, 51, pp 619-637); terpenes such as 1,8-cineole, menthone, limonene and nerolidol (Yamane, J. Pharmacy & Pharmacology, 1995, 47, pp 978-989); Azone and Transcutol (Harrison et al, Pharmaceutical Res., 1996, 13, pp 542-546); and oleic acid, polyethylene glycol and propylene glycol (Singh et al, Pharmazie, 1996, 51, pp 741-744) are known to improve skin penetration of an active ingredient.

Levels of penetration of a compound of the present invention can be determined by techniques known to those of skill in the art. For example, radiolabelling of the active compound, followed by measurement of the amount of radiolabelled compound absorbed by the skin enables one of skill in the art to determine levels of the composition absorbed using any of several methods of determining skin penetration of the test compound. Publications relating to skin penetration studies include W. G. Reinfenrath and G. S. Hawkins, The Weanling Yorkshire Pig as an Animal Model for Measuring Percutaneous Penetration, in Swine in Biomedical Research, M. E. Tumbleson ed., Plenum, New York, 1986; and G. S. Hawkins, Methodology for the Execution of In Vitro Skin Penetration Determinations, in Methods for Skin Absorption, B. W. Kemppainen and W. G. Reifenrath eds., CRC Press, Boca Raton, 1990, pp 67-80; and W. G. Reifenrath, Cosmetics & Toiletries, 1995, 110, pp 3-9.

For some applications, it is preferable to administer a long acting form of a composition of the present invention using formulations known in the arts, such as polymers. Moreover, the compounds of the present invention can be incorporated into a dermal patch (H. E. Junginger, Acta Pharmaceutica Nordica, 1992, 4, pp 117; Thachatodi et al, Biomaterials, 1995, 16, pp 145-148; R. Niedner, Hautarzt, 1998, 39, pp 761-766) or a bandage according to methods known in the art, to increase the efficiency of delivery of the drug to the areas to be treated.

Optionally, the topical formulations of this invention can have additional excipients, for example, preservatives such as methylparaben, benzyl alcohol, sorbic acid or quaternary ammonium compound, stabilizers such as EDTA, antioxidants such as butylated hydroxytoluene or butylated hydroxyanisole, and buffers such as citrate and phosphate.

The pharmaceutical composition can be administered in an oral formulation in the form of tablets, capsules or solutions. An effective amount of the oral formulation is administered to patients 1 to 3 times daily until the symptoms of the proliferative disease, cancer or photoageing etc. are alleviated. The effective amount of a compound of the present invention depends on the age, weight and condition of a patient. In general, the daily oral dose of a compound of the present invention is less than 1200 mg, and more than 100 mg. The preferred daily oral dose is about 300-600 mg. Oral formulations are conveniently presented in a unit dosage form and may be prepared by any method known in the art of pharmacy. The composition may be formulated together with a suitable pharmaceutically acceptable carrier into any desired dosage form. Typical unit dosage forms include tablets, pills, powders, solutions, suspensions, emulsions, granules, capsules, and suppositories. In general, the formulations are prepared by uniformly and intimately bringing into association the compound of the present invention with liquid carriers or finely divided solid carriers or both, and as necessary, shaping the product. The active ingredient can be incorporated into a variety of basic materials in the form of a liquid, powder, tablets or capsules to give an effective amount of active ingredient.

Other therapeutic agents suitable for use herein are any compatible drugs that are effective for the intended purpose, or drugs that are complementary to the formulation. As an example, the treatment with a formulation of this invention can be combined with other treatments such as a topical treatment with corticosteroids, calcipotriene, coal tar preparations, a systemic treatment with methotrexate, retinoids, cyclosporin A and photochemotherapy. The combined treatment is especially important for treatment of an acute or a severe skin proliferation disease. The formulation utilized in a combination therapy may be administered simultaneously, or sequentially with the other treatment, such that a combined effect is achieved.

### SYNTHETIC EXAMPLES

### Example 1

### (Z)-But-2-enedioic acid mono-(11-methoxycarbonyl-4,4,6a,6b,8a,11,14b-heptamethyl-14-oxo-1,2,3,4,4a,5,6,6a,6b,7,8,8a,9,10,11,12,12a,14,14a, 14b-icosahydro-picen-3-yl) ester (YP013)

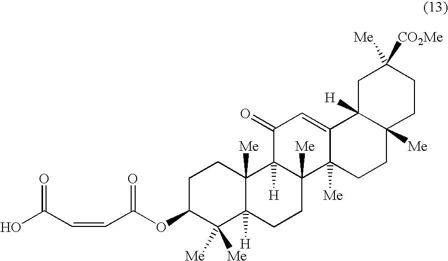

Maleic anhydride (401 mg, 4.09 mmol) was added to a solution of methyl glycyrrhetinate (150 mg, 0.310 mmol) in toluene (4 ml) at room temperature. The solution was then heated to reflux (110° C.) and stirred for 16 hours. After this time, the solution was cooled and diluted with DCM (10 ml). The organic solution was then washed with 2M HCl (10 ml), the aqueous layer extracted with DCM (3×10 ml) and the organics dried (MgSO₄). After filtration, the solution was concentrated under vacuum and purified by flash column chromatography (2:1 DCM:EtOAc to 1:1 DCM:EtOAc) to give title compound YP013 as a white solid (100 mg, 55% yield).

LCMS purity 89.63% (583.4=[M+H]⁺).

¹H NMR (250 MHz, CDCl₃) δ 6.48 (1H, d, J=12.8 Hz, CO₂HCH═CH), 6.36 (1H, d, J=12.8 Hz, CH═CHCO₂C), 5.66 (1H, s, C[O]CH═C), 4.70 (1H, m, CO₂CHCH₂), 3.68 (3H, s, CO₂CH₃), 2.88 (1H, m, ═CCHCH₂), 2.36 (1H, s, C[O]CHC) and 2.14-0.70 (40H, m, steroid CH, CH₂ and CH₃ protons). The coupling constant J=12.8 Hz shows that compound YP013 is the Z form (see “Spectroscopic methods in organic chemistry” by D. H. Williams and I. Fleming, McGraw-Hill Book Company, 4^{th} edition, 1989).

### Example 2

### Phthalic acid mono-(11-methoxycarbonyl-4,4,6a,6b,8a,11,14b-heptamethyl-14-oxo-1,2,3,4,4a,5,6,6a,6b,7,8,8a,9, 10,11,12,12a, 14,14a, 14b-icosahydro-picen-3-yl) ester (YP015)

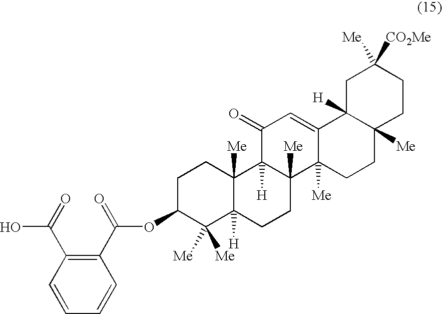

Phthalic anhydride (468 mg, 3.16 mmol) was added to a solution of methyl glycyrthetinate (300 mg, 0.62 mmol) and DMAP (307 mg, 2.52 mmol) in pyridine (10 ml) at room temperature. The solution was then heated to 90° C. and stirred for 16 hours. After this time, the solution was cooled and diluted with DCM (20 ml). The organic solution was then washed with 2M HCl (20 ml), the aqueous layer extracted with DCM (3×20 ml) and the organics dried (MgSO₄). After filtration, the solution was concentrated under vacuum and purified by flash column:chromatography (10:1 heptane:EtOAc to 1:1 heptane:EtOAc) to give title compound YP015 as a white solid (280 mg, 710% yield).

LCMS purity 95.7% (631=[M−H]⁻).

¹H NMR (250 MHz, MeOD) δ 7.80-7.56 (4H, m, Ar—H), 5.58 (1H, s, C[O]CH═C), 4.74 (1H, m, CO₂CHCH₂), 3.68 (3H, s, CO₂CH₃), 2.82 (1H, m, ═CCHCH₂), 2.52 (1H, s, C[O]CHC) and 2.20-0.74 (41H, m, steroid CH, CH₂ and CH₃ protons).

### Example 3

### 2,4a,6a,6b,9,9,12a-Heptamethyl-13-oxo-10-(3-oxo-butyryloxy)-1,2,3,4,4a,5,6, 6a,6b, 7,8,8a,9,10,11,12, 12a,12b,13,14b-icosahydro-picene-2-carboxylic acid (YP016)

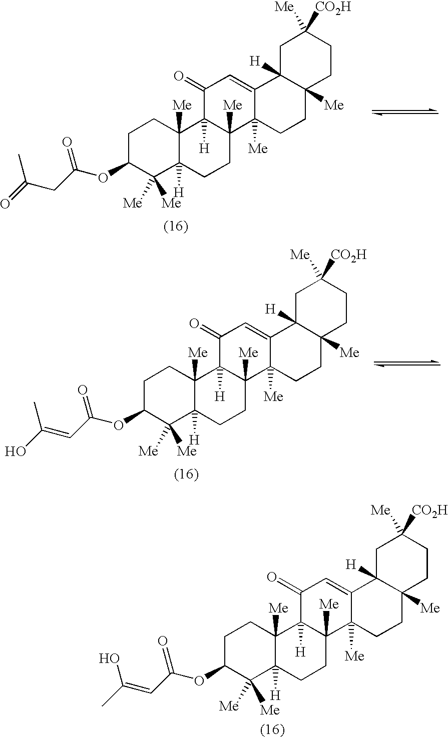

N-hydroxysuccinimidyl acetoacetate (614 mg, 3.09 mmol) was added to a solution of glycyrrhetinic acid (500 mg, 1.06 mmol) in toluene (10 ml) and DMF (2 ml) at room temperature. The solution was then heated to reflux (110° C.) and stirred for 16 hours. After this time, the solution was cooled and diluted with DCM (20 ml). The organic solution was then washed with 2M HCl (20 ml), the aqueous layer extracted with DCM (3×20 ml) and the organics dried (MgSO₄). After filtration, the solution was concentrated under vacuum and purified by flash column chromatography (10:1 heptane:EtOAc to 1:1 heptane:EtOAc) to give title compound YP016 as a yellow solid (90 mg, 15% yield).

LCMS purity 81.17% (556=[M+H]⁺).

¹H NMR (250 MHz, CDCl₃) δ 5.68 (1H, s, C[O]CH═C), 4.58 (1H, m, CO₂CHCH₂), 3.44 (3H, s, C[O]CH₂CO₂CH), 2.82 (1H, m, ═CCHCH₂), 2.34 (1H, s, C[O]CHC), 2.26 (3H, s, CH₃C[O]CH₂) and 2.22-0.70 (40H, m, steroid CH, CH₂ and CH₃ protons).

### Example 4

### Phthalic acid mono-(11-carboxy-4,4,6a,6b,8a,11,14b-heptamethyl-14-oxo-1,2,3, 4,4a,5,6,6a,6b,7,8,8a,9,10,11,12,12a,14,14a, 14b-icosahydro-picen-3-yl) ester (YP017)

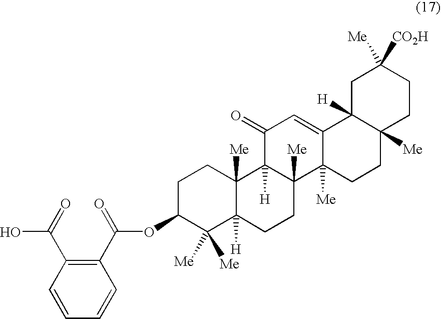

Phthalic anhydride (821 mg, 5.54 mmol) was added to a solution of glycyrrhetinic acid (500 mg, 1.06 mmol) and DMAP (250 mg, 2.05 mmol) in pyridine (15 ml) at room temperature. The solution was then heated to 90° C. and stirred for 16 hours. After this time, the solution was acidified with 2M HCl (20 ml) and a white precipitate formed. After filtration, the crude solid was triturated with DCM (100 ml) then MTBE (100 ml) to give title compound YP017 as a white solid (50 mg, 8% yield).

LCMS purity 97.56% (617=[M−H]⁻, 619=M+H]⁺).

¹H NMR (250 MHz, CDCl₃) δ 7.98 (1H, m, Ar—H), 7.64-7.46 (4H, m, Ar—H), 5.68 (1H, s, C[O]CH═C), 4.84 (1H, m, CO₂CHCH₂), 2.92 (1H, m, ═CCHCH₂), 2.38 (1H, s, C[O]CHC) and 2.22-0.80 (40H, m, steroid CH, CH₂ and CH₃ protons).

### Example 5

### 2,2-Dimethyl-succinic acid 4-((3S,4aR,6aR,6bS,8aS,11S,12aR,14aR,14bS)-11-carboxy-4,4,6a,6b,8a,11,14b-heptamethyl-14-oxo-1,2,3,4,4a,5,6,6a,6b,7,8,8a,9,10,11,12, 12a,14,14a, 14b-icosahydro-picen-3-yl) ester (YP022)

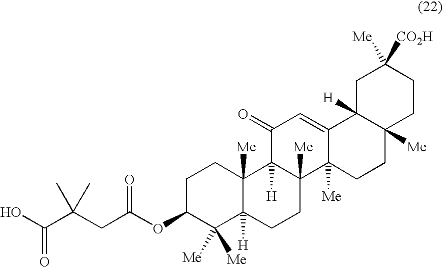

A mixture of glycyrrhetinic acid (0.5 g, 1.1 mmol), 2,2-dimethylsuccinic anhydride (1.5 g, 11 mmol) and DMAP (130 mg, 1.1 mmol) in pyridine (15 ml) was heated at 110° C. for 16-20 hours, cooled, added to 2M aqueous HCl solution (200 ml) and the solids collected. After drying, the crude solid was purified by elution through a pad of silica using 0-10% methanol in DCM as eluent to give title compound YP022 as a white solid (100 mg).

LCMS purity 97.9% (597=[M−H]⁻).

### Example 6

### trans-Cyclohexane-1,2-dicarboxylic acid mono-((3S,4aR,6aR,6bS,8aS,11S, 12aR,14aR,14bS)-11-carboxy-4,4,6a,6b,8a,11,14b-heptamethyl-14-oxo-1,2,3,4,4a,5,6,6a,6b, 7,8,8a,9,10,11,12,12a,14,14a, 14b-icosahydro-picen-3-yl) ester (YP023, mixture of stereoisomers)

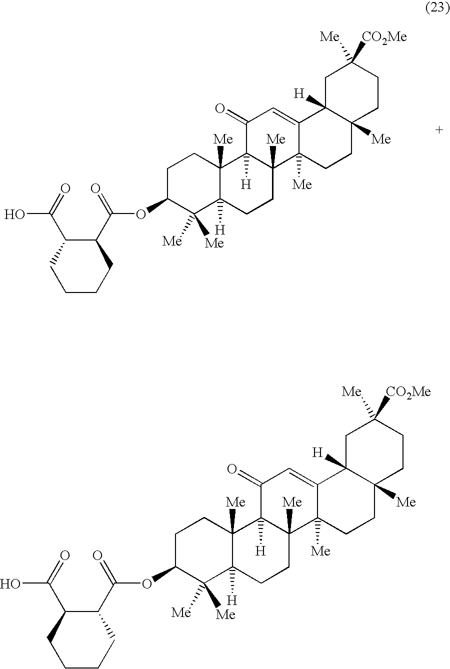

Glycyrrhetinic acid methyl ester (500 mg, 1.0 mmol) was dissolved in pyridine (10 ml). DMAP (130 mg, 4.5 mmol) and trans-1,2-cyclohexanedicarboxylic anhydride (1.7 g, 11 mmol) were added and the mixture heated to 95° C. for 16 hours. After cooling, the mixture was added to 2M aq. HCl solution (150 ml) and the solids collected by filtration. The gummy solid was dissolved in a mixture of DCM and methanol and dried (MgSO₄). Purification was carried out by elution through a short pad of silica with 1:1, then 2:2, i-PrOAc: heptane to give title compound YP023 an off-white solid (605 mg).

LCMS purity 78.5% (637=[M−H]⁻).

¹H NMR (400 MHz, CDCl₃) δ 5.65 (1H, s), 4.45 (1H, m), 3.65 (3H, s), 2.8-2.6 (3H, m), 2.4-0.7 (50H, m).

### Example 7

### cis-Cyclohexane-1,2-dicarboxylic acid mono-((3S,4aR,6aR,6bS,8aS,11S, 12aR,14aR,14bS)-11-carboxy-4,4,6a,6b,8a,11,14b-heptamethyl-14-oxo-1,2,3,4,4a,5,6,6a,6b, 7,8,8a,9,10,11,12, 12a, 14, 14a, 14b-icosahydro-picen-3-yl) ester (YP024, mixture of stereoisomers)

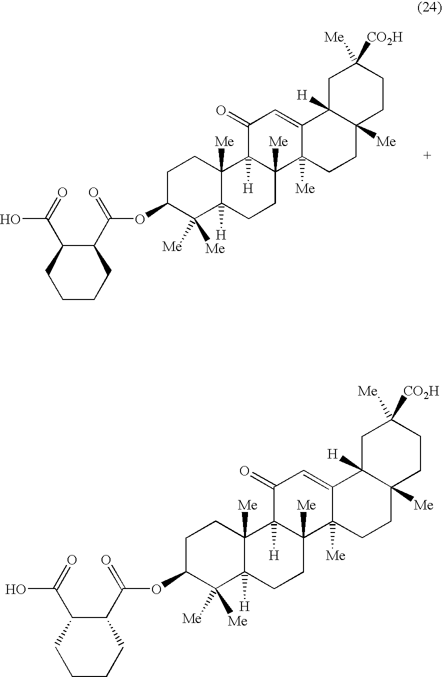

A mixture of glycyrrhetinic acid (0.5 g, 1.1 mmol), cis-1,2-cyclohexanedicarboxylic anhydride (1.7 g, 11 mmol) and DMAP (130 mg, 4.5 mmol) in pyridine (15 ml) was heated at 100° C. for 16-20 hours to give a yellow solution. After cooling, the mixture was added to 2M aqueous HCl solution (200 ml) and the solid collected and dried. Elution through a silica pad with 1:11-propyl acetate:heptane followed by 2:11-propyl acetate:heptane gave title compound YP024 as an off-white solid (135 mg).

LCMS purity 88.2% (623=[M−H]⁻).

¹H NMR (400 MHz, CDCl₃) δ 5.68 (1H, s), 4.55 (1H, dd), 2.90-2.70 (6H, m), 2.45 (1H, s), 2.45-0.70 (46H, m).

### Example 8

### Cyclopent-1-ene-1,2-dicarboxylic acid mono-((3S,4aR,6aR,6bS,8aS,11S,12aR, 14aR,14bS)-11-carboxy-4,4,6a,6b,8a,11,14b-heptamethyl-14-oxo-1,2,3,4,4a,5,6,6a,6b,7,8, 8a,9,10,11,12,12a,14,14a, 14b-icosahydro-picen-3-yl) ester (YP026)

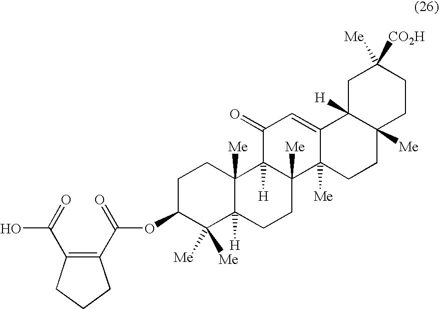

A mixture of glycyrrhetinic acid (0.5 g, 1.1 mmol), cyclopentene-1,2-dicarboxylic acid anhydride (1.0 g, 7.2 mmol) and DMAP (130 mg, 4.5 mmol) in pyridine (15 ml) was heated at 100° C. for 16-20 hours to give a black solution. After cooling, the mixture was added to 2M aqueous HCl solution (200 ml) and the dark solid collected and dried. The crude solid was purified by elution through a 10 g silica cartridge on the FlashMaster™ using 1:11-propyl acetate:heptane to give title compound YP026 as a pure, brown solid (68 mg).

LCMS purity 91.5% (607=[M−H]⁻).

¹H NMR (400 MHz, CDCl₃) δ 5.85 (1H, s), 4.70 (1H, dd), 3.0-2.8 (4H, m), 2.40 (1H, s), 2.40-0.80 (43H, m).

### Example 9

### 4-Fluoro-phthalic acid 1-((3S,4aR,6aR,6bS,8aS,11S,12aR,14aR,14bS)-11-carboxy-4,4,6a,6b,8a,11,14b-heptamethyl-14-oxo-1,2,3,4,4a,5,6,6a,6b,7,8,8a,9,10,11,12, 12a,14,14a,14b-icosahydro-picen-3-yl) ester and 4-Fluoro-phthalic acid 2-((3S,4aR,6aR,6bS, 8aS,11S,12aR,14aR,14bS)-11-carboxy-4,4,6a,6b,8a,11,14b-heptamethyl-14-oxo-1,2,3,4,4a, 5,6,6a,6b,7,8,8a,9,10,11,12,12a,14,14a,14b-icosahydro-picen-3-yl) ester (YP032, Mixture of Regioisomers)

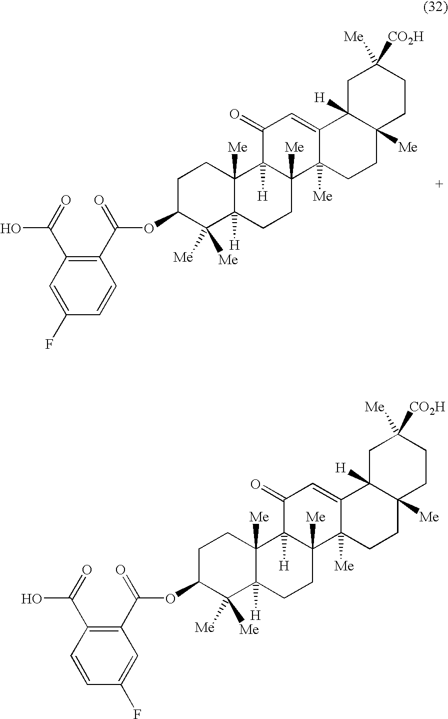

A mixture of glycyrrhetinic acid (1.0 g, 2.2 mmol) and 4-fluorophthalic anhydride (1.0 g, 2.7 mmol) in toluene (50 ml) was heated at reflux for 48 hours, when DMF (5 ml) was added to dissolve some residual solid and heating was continued for 24 hours. HPLC showed only a small conversion to new product and DMAP (27 mg, 0.22 mmol) was added and heating continued for 3 days, when the mixture was cooled. Filtration gave no solid, so the mixture was concentrated and purified by silica chromatography (eluent: 0-5% methanol in DCM) to give title compounds YP032, a mixture of regioisomers, as an off-white solid (220 mg).

LCMS purity 97.9% (635.5=[M−H]⁻).

¹H NMR (400 MHz, CDCl₃) (selected resonances from the crude mixture of isomers) δ 8.11 (1H, m, single compound), 7.8 (1H, m, single compound), 7.7 (1H, m, single compound), 7.35-7.1 (3H, m, two compounds), 5.7 (1H, s, two compounds), 4.7 (1H, m, two compounds), 3.90 (1H, d, two compounds), 2.23 (1H, br s, two compounds), 2.2-0.8 (41H, m, two compounds).

### Example 10

### Pyrazine-2,3-dicarboxylic acid mono-((3S,4aR,6aR,6bS,8aS,11S,12aR,14aR, 14bS)-11-methoxycarbonyl-4,4,6a,6b,8a, 11,14b-heptamethyl-14-oxo-1,2,3,4,4a,5,6,6a,6b,7,8, 8a,9,10, 11,12,12a,14,14a, 14b-icosahydro-picen-3-yl) ester (YP034)

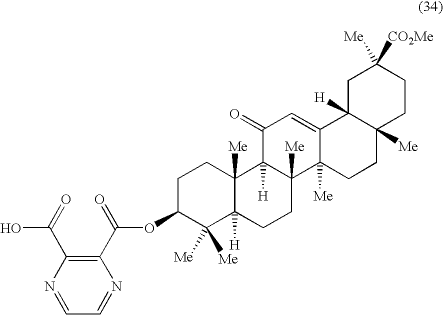

Glycyrrhetinic acid methyl ester (1.0 g, 2.0 mmol) was stirred in toluene (25 ml) and 2,3-pyrazine-dicarboxylic anhydride (1.7 g, 11.5 mmol) added. The mixture was heated at 110° C. for 16-20 hours. After cooling, the mixture was added to water (200 ml) and extracted into i-propyl acetate (3×70 ml), which was dried (MgSO₄) and concentrated to give the crude adduct. Purification was effected using silica chromatography (eluent: 0-10% methanol in DCM) to give the title compound as a yellow solid (500 mg).

LCMS purity 90.5% (633=[M−H]⁻).

¹H NMR (400 MHz, CDCl₃) δ 8.78 (1H, br), 8.70 (1H, br), 5.68 (1H, s) 4.88 (1H, dd, J=11.6 Hz, 4.8 Hz), 3.62 (3H, s), 2.80 (1H, br d), 2.35 (1H, s), 2.1-0.6 (40H, m).

### Biological Examples

### Example 11

### Effect of 18β-Glycyrrhetinic Acid Derivatives on Normal Human

Keratinocyte Cell Proliferation

An assay was undertaken to determine the effect of five 18β-glycyrrhetinic acid derivatives (carbenoxolone sodium and compounds YP013, YP015, YP016 and YP017 of the present invention) on normal human keratinocyte cell proliferation.

Normal human keratinocyte (NHEKa) cells were cultured in the presence and absence of set concentrations of the five 18β-glycyrrhetinic acid derivatives, in multi-well assay plates (96), over a five-day period. The cells, seeded at a density of 5,000 or 2,500 cells per well, were cultured in keratinocyte growth medium (KGM) (Cascade Biologics), to which retinol and various concentrations of the 18β-glycyrrhetinic acid derivatives dissolved in KGM and DMSO were added. Control treatments included KGM only, KGM and retinol, and KGM and DMSO.

After the five-day period, cell proliferation was determined by assay of ATP levels (directly associated with cell density) using a commercially available kit (CellTitre-Glo, Promega). In the presence of suitable controls, reduced proliferation (cell growth measured by ATP generation) indicates an anti-proliferative effect of a given 18β-glycyrrhetinic acid derivative.

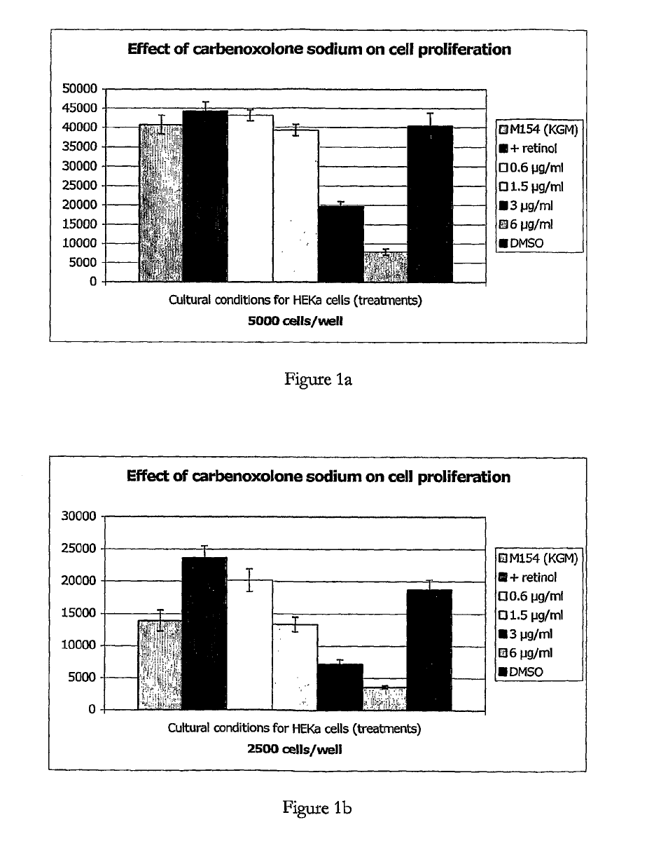

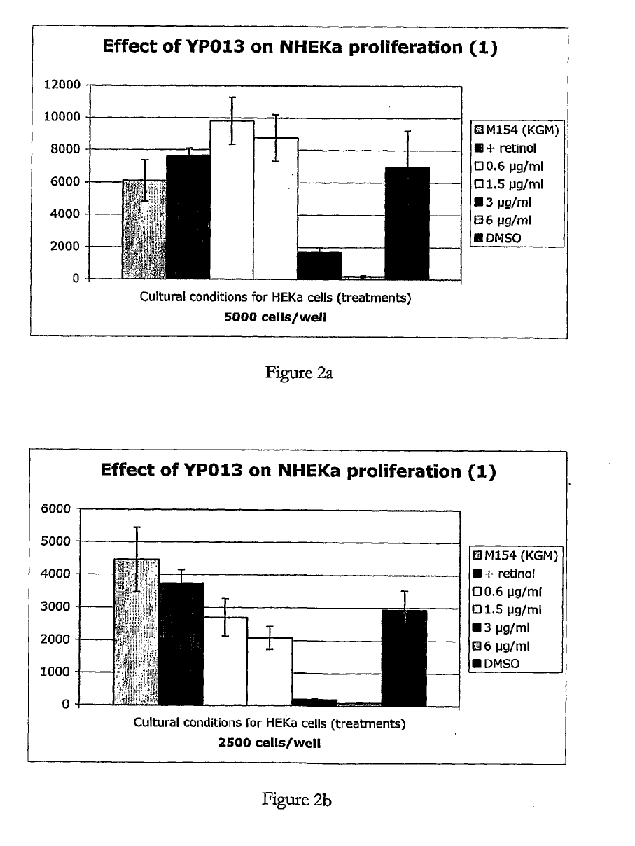

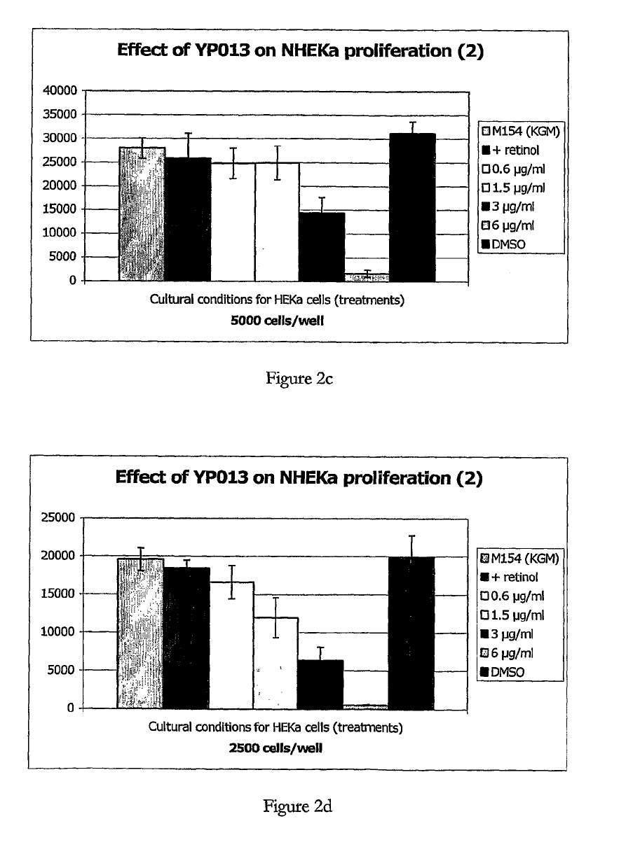

FIGS. 1a and 1b show the results obtained for carbenoxolone sodium. FIGS. 2a-d, 3a-d, 4a-d and 5a-d show the results obtained for four compounds of the present invention, namely YP013, YP015, YP016 and YP017 respectively. For the compounds of the present invention, two repeats of the assay of ATP levels were performed, which are illustrated in Figures a/b (labelled (1)) and c/d (labelled (2)) respectively.

As can be seen from FIGS. 1a and 1b, carbenoxolone sodium suppresses NHEKa cell proliferation under the conditions tested.

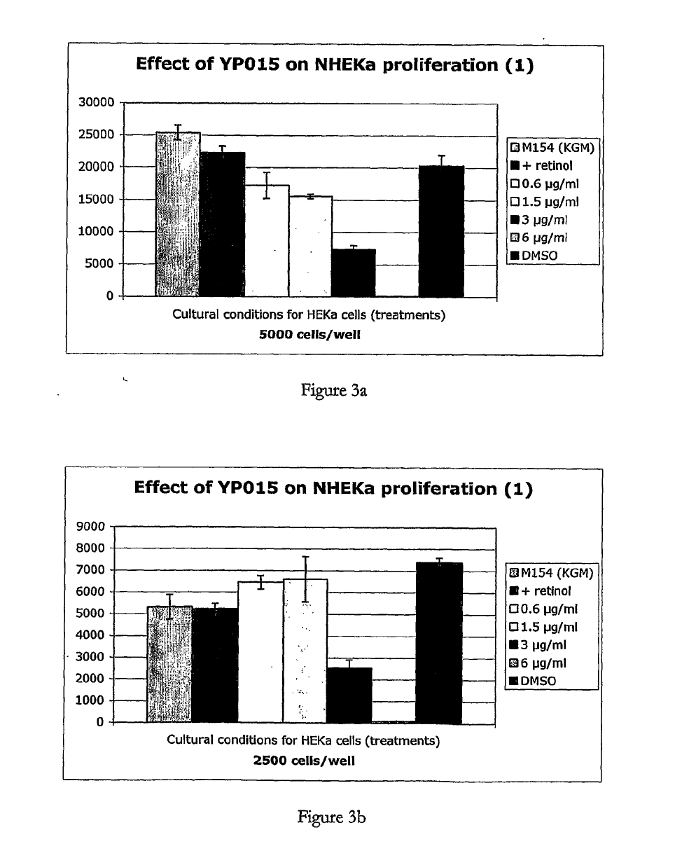

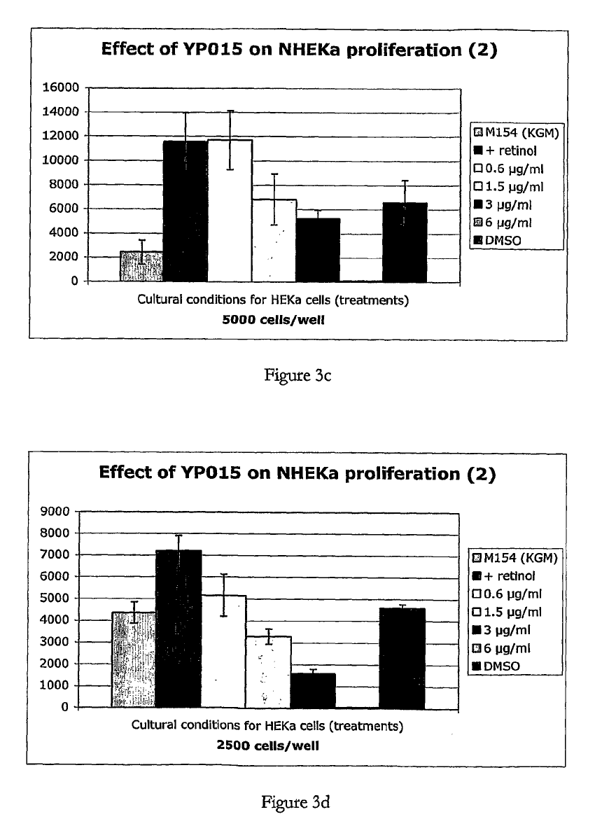

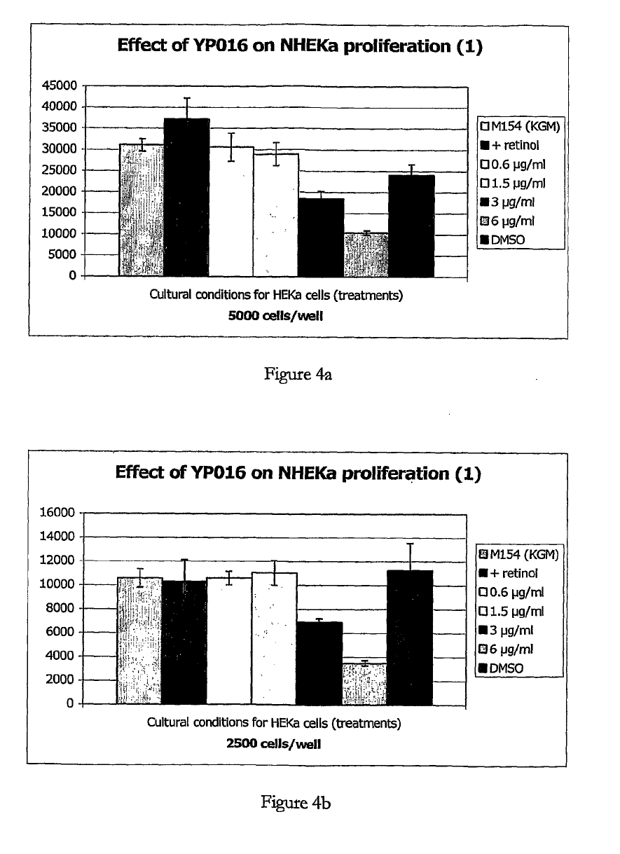

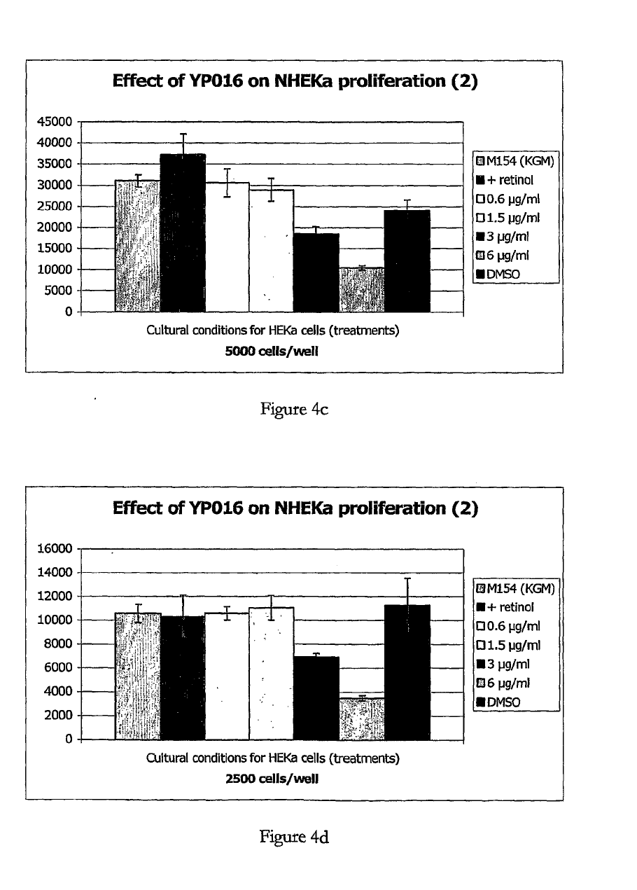

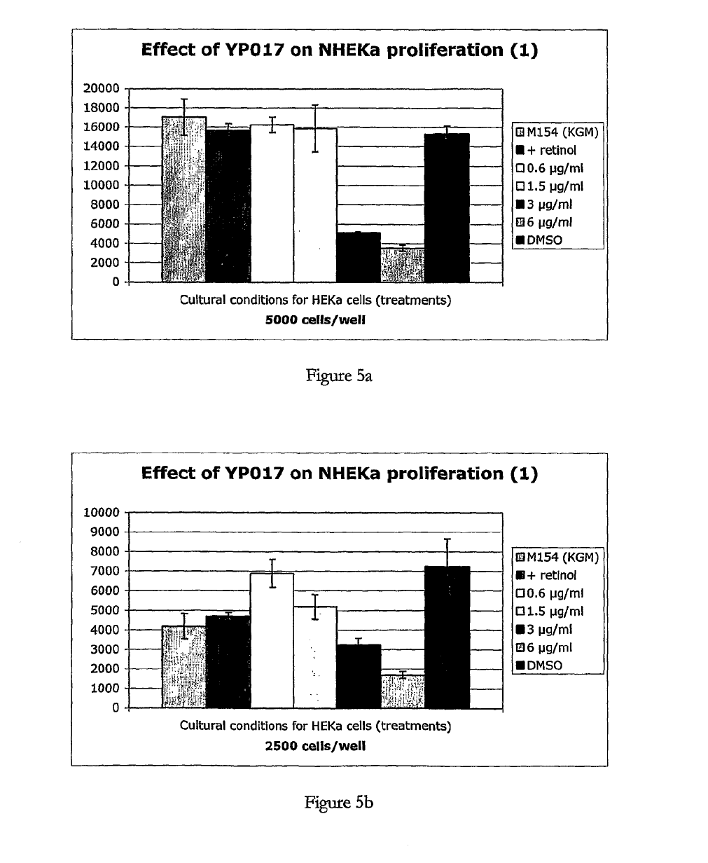

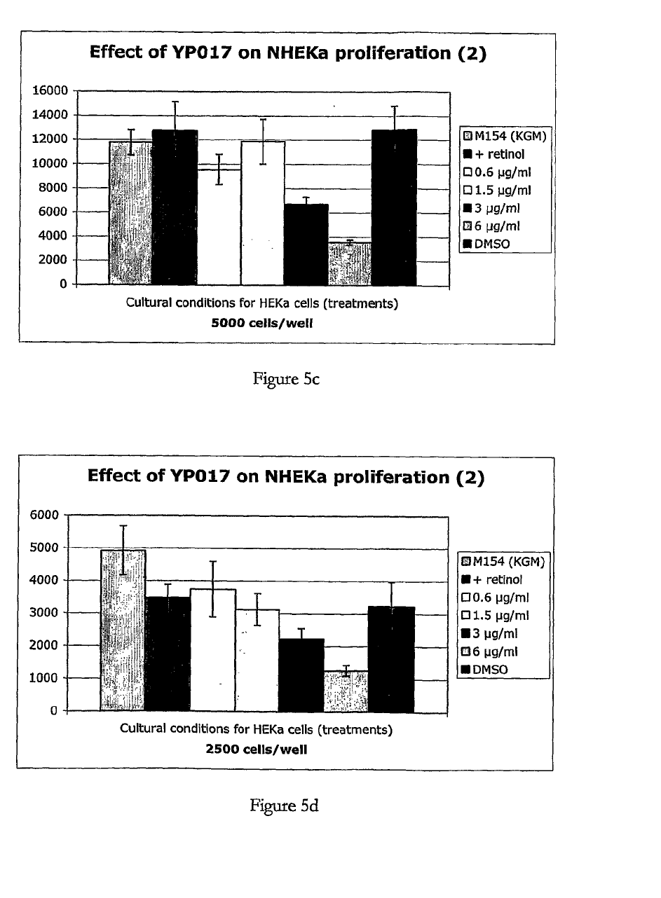

As can be seen from FIGS. 2-5, in both repeats of the assay conducted, ATP levels were found to be significantly lower following treatment of the cells with the four compounds of the present invention compared to the controls, in a manner dependent on the concentration of the compounds of the present invention. The Figures also indicates that at least compounds YP013 and YP015 have anti-proliferative properties, which are more potent than those of carbenoxolone sodium at equal concentrations.

### Example 12

### Effect of 18β-glycyrrhetinic Acid Derivatives on Normal Human Keratinocyte Cell Proliferation

Carbenoxolone and ten 18β-glycyrrhetinic acid derivatives of the present invention (YP013, YP015, YP016, YP017, YP022, YP023, YP024, YP026, YP032 and YP034) were tested using the NHEKa cell viability assay of example 11 to determine their anti-proliferative activity at various concentrations.

In the NHEKa cell viability assay of example 11, specific inhibitors were tested at a range of concentrations on a single plate including the appropriate controls. To obtain a direct comparison of each inhibitor compared with carbenoxolone, the design of the assay was amended, so that each plate contained a range of different inhibitors at the same concentration compared to carbenoxolone. This removed inter-plate variation when comparing inhibitors used at the same concentration.

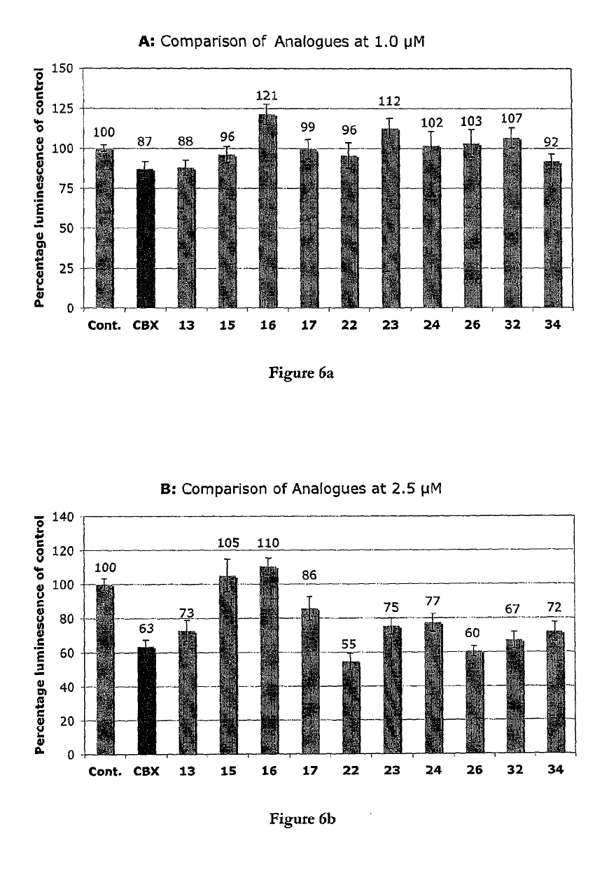

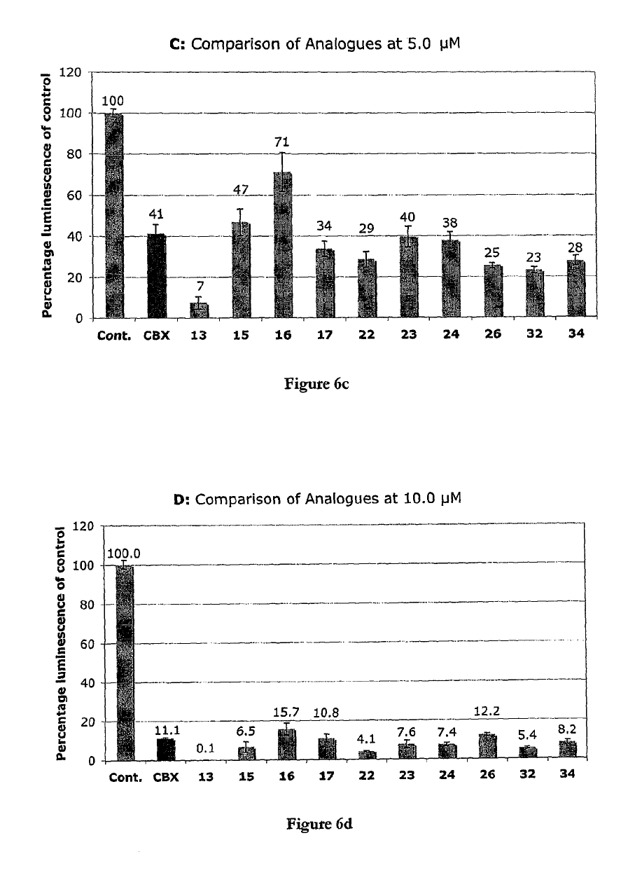

NHEKa cells, seeded at 5,000 cells per well of a multi-well plate (96), were cultured in an optimised NHEK growth medium (Medium 154, Cascade Biologics) for 5 days, at which point the amount of ATP present in the lysed cell suspension was indirectly quantified. The medium (200 μl in each well) was replaced after 24 and 72 hours of growth with fresh pre-warmed medium containing retinol (500 nM final concentration) and the indicated inhibitor, consequently the length of treatment was 72 hours. The inhibitors, carbenoxolone (CBX) and ten 18β-glycyrrhetinic acid derivatives of the present invention (YP013, YP015, YP016, YP017, YP022, YP023, YP024, YP026, YP032 and YP034) were included at final concentrations of 1.0 μM, 2.5 μM, 5.0 μM and 10.0 μM. The amount of ATP quantified using the CellTitre-Glo kit (Promega) is an indirect indicator of the number of viable cells present at the end-point of the experiment and was measured as luminescence, presented as a percentage of the control culture (medium+retinol). The results, shown in FIGS. 6a-d, illustrate an average of at least 3 repeats with error bars indicating the standard error.

At the lowest concentration of the inhibitors tested, 1.0 μM, there was very little change in luminescence observed (FIG. 6a). When the concentration was increased 2.5-fold, a number of the treated cell cultures showed significantly less luminescence compared to the controls (FIG. 6b). Carbenoxolone treated cells were among these, along with derivatives YP022 and YP026. Doubling the concentration of inhibitor resulted in much greater effects on luminescence, exemplified by only 41% luminescence detected in carbenoxolone treated cells compared to the untreated control (FIG. 6c). Interestingly, carbenoxolone no longer appeared to be the most active compound. Derivative YP013 resulted in a 93% reduction in luminescence compared to the control and 34% compared to carbenoxolone treated cells. The effect of the highest concentration tested, 10.0 μM, is illustrated in FIG. 6d. At 10 μM, all inhibitors tested resulted in greater than an 84% reduction in luminescence. Derivative YP013 demonstrated the greatest activity by reducing luminescence on average 1000 times.

The results presented here demonstrate that all ten 18β-glycyrrhetinic acid derivatives of the present invention tested reduce end-point luminescence compared to control. Eight of the ten derivatives of the present invention also possess a greater ability than carbenoxolone to reduce end-point luminescence. Because the amount of luminescence is dependent on the concentration of ATP, decreased luminescence is related to a quantifiable decrease in ATP. The amount of ATP is also likewise dependent on the number of viable cells present, therefore, reduced ATP levels translate into reduced cell viability. It can therefore be concluded that treatment with the derivatives tested here results in reduced viable cell counts.

### Example 13

### Effect of 18β-glycyrrhetinic Acid Derivatives on Viability of Normal Human Keratinocytes

An assay was undertaken to assess the viability of normal human keratinocytes when subjected to treatment with carbenoxolone and six 18β-glycyrrhetinic acid derivatives of the present invention (YP013, YP015, YP016, YP023, YP024 and YP026).

The cells used were normal human keratinocytes (NHEKa). All cells were cultured in T75 flasks according to the supplier's guidelines for maintenance of stocks. Frozen stocks of cells that had gone through two passages were seeded into T25 flasks at a seeding density of 2.5×10³ cells per cm². The cells were then left to grow over 5 days with media changes being carried out every two days.

Cell culture treatment media were prepared for all the test compounds at four concentrations, namely 1 μM, 2.5 μM, 5 μM and 10 μM (final concentration). Control treatments on cells of normal media (no treatment) and DMSO were also carried out.

All treatment media were sterilised by filtration and added to the T25 flasks after removal of spent media. The cells were then incubated at 37° C. for 24 hours. The treatment media were removed from the T25 flasks and the cells washed with 5 ml of PBS before 1 ml of Trypsin/EDTA (TE) (Cascade, Mansfield, UK) solution was added to the flaks and left for approximately 8-10 minutes for the cells to lift off the growth surface. The trypsin was neutralised with 3 ml Trypsin Neutraliser (TN) (Cascade). The cells were then harvested and transferred to a separate 15 ml centrifuge tube for each treatment regime. The cells were spun at 180×g for 7 minutes and the medium was removed.

The cells were resuspended in 1 ml of ice cold PBS before being transferred to a microcentrifuge tube and spun at 200×g for 2 minutes. The PBS was removed and the cells were resuspended in 100 μl of binding buffer (10 mM Hepes, 140 mM NaCl, 2.5 mM CaCl₂, pH 7.4) before the addition of 5 μl of a propidium iodide (PI) staining solution (BD Biosciences, Cowley, UK). The cells were incubated at 20° C. for 5 minutes before the addition of 300 μl of binding buffer followed by immediate analysis by flow cytometry. Cells taking up the PI stain were dead, cells not staining with PI were viable. The level of staining within the cells could be viewed by flow cytometry.

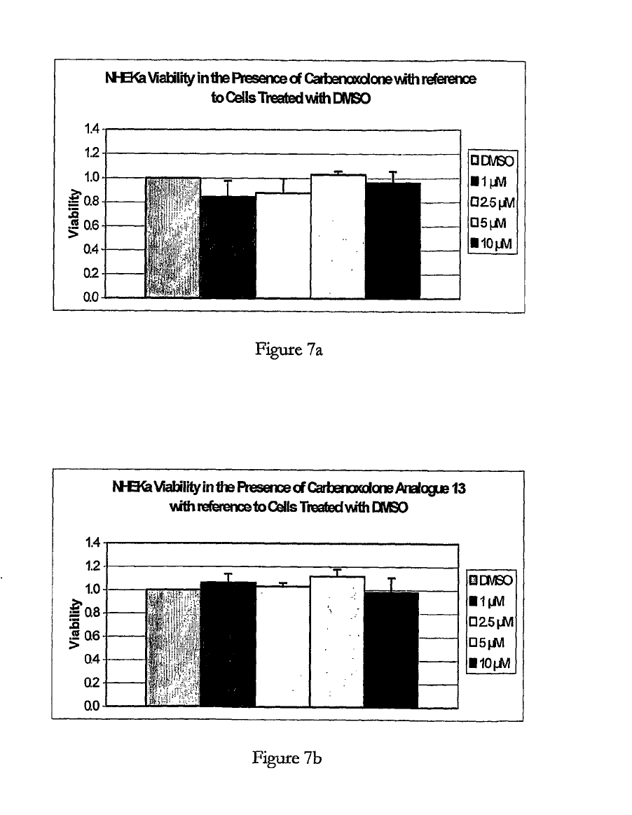

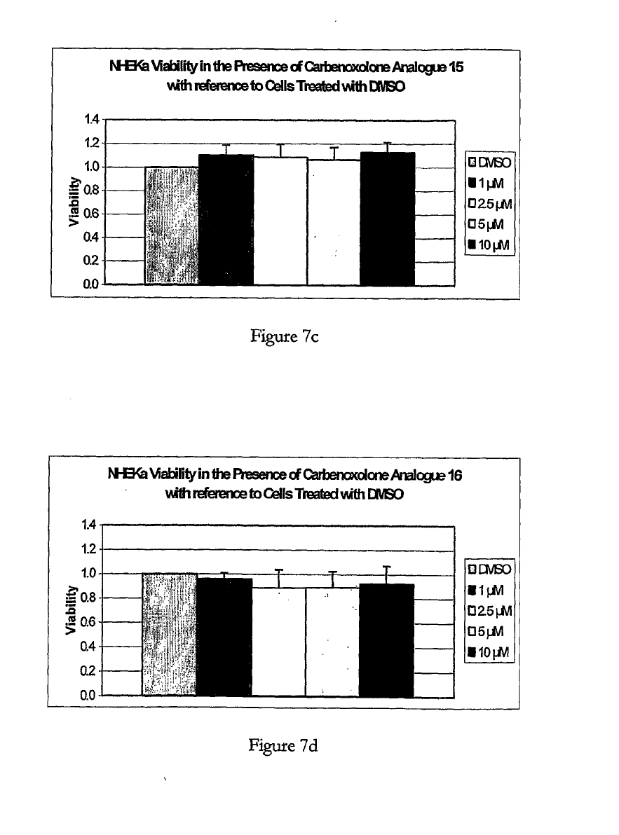

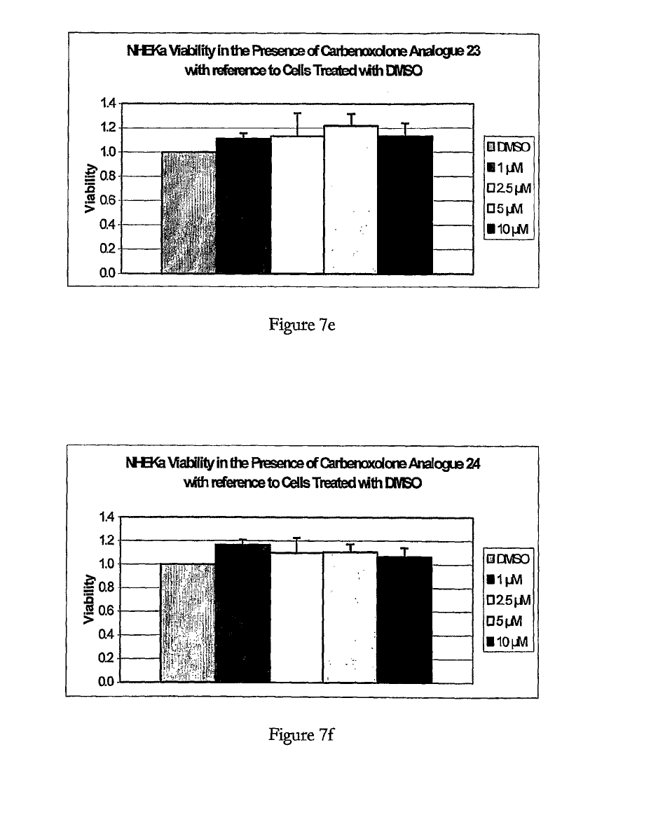

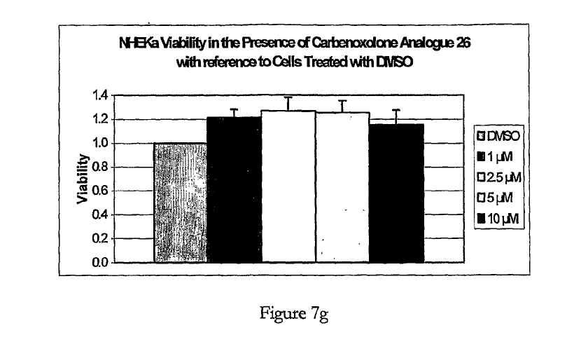

The cell viability following treatment was recorded as a proportion of viable cells when referenced to the proportion of viable cells when only DMSO had been applied. This type of analysis takes account of the natural variability in the growth and survival of different batches of cells. The results the viability experiments are shown in FIGS. 7a-g. Data presented are from three repeats +SEM.

As can be seen from FIG. 7a, a slight decrease in viability is indicated at the 1 μM and 2.5 μM concentrations of carbenoxolone when the SEM is considered, indicating that low levels of carbenoxolone may be slightly cytotoxic to NHEKa cells over a 24 hour period. FIGS. 7b-d show that no decrease in viability is indicated at any of the four concentrations of compounds YP013, YP015 and YP016, indicating that they do not appear to be cytotoxic to NHEKa cells over a 24 hour period. A slight increase in viability is indicated at all four concentrations of compounds YP023, YP024 and YP026 (see FIGS. 7e-g). Hence these compounds also do not appear to be cytotoxic to NHEKa cells over a 24 hour period and may even promote cell survival.

Therefore, with the possible exception of carbenoxolone, none of the compounds tested appears to decrease the viability of NHEKa cells over an incubation time of 24 hours. Carbenoxolone itself appears to have a slightly cytotoxic effect at the 1 μM and 2.5 μM concentration.

### Example 14

### The Effect of 18β-Glycyrrhetinic Acid Derivatives on Recombinant Human Retinol Dehydrogenase Enzyme 2 (hRoDH-E2) Activity In Vitro

The effect of carbenoxolone and eight 18β-glycyrrhetinic acid derivatives of the present invention (YP013, YP015, YP016, YP017, YP022, YP024, YP026 and YP032) on recombinant human retinol dehydrogenase enzyme 2 activity was assessed using a spectrometric assay to determine whether these compounds can inhibit the activity of recombinant human retinol dehydrogenase enzyme 2 in vitro.

A reaction mix was prepared, consisting of a buffered solution containing excess retinol (10 mM HEPES, 150 mM KCl, 2 mM EDTA, pH 8.0; 0.02% Tween 80; 125 μM retinol). Preliminary experiments concluded that sufficient cofactor was present in the crude enzyme source, therefore, this was not supplemented into the reaction buffer. The enzyme source was provided by the BioCatalysis Centre, University of Exeter, as a crude sonicated supernatant (SS) from harvested E. coli RIL cells transfected with the hRoDH-E2 expression vector. The sonicated supernatant was prepared by resuspending 1 g cell paste in 10 ml distilled H₂O containing broad-spectrum protease inhibitors (Roche Protease Inhibitor Cocktail tablets). The cells were then mechanically disrupted by sonication on ice. Samples from E. coli RIL cultures minus the expression vector (-hRoDH-E2) were also provided as negative controls. The inhibitors, carbenoxolone (Sigma Aldrich) and eight 18β-glycyrrhetinic acid derivatives of the present invention (YP013, YP015, YP016, YP017, YP022, YP024, YP026 and YP032), dissolved in DMSO, were added so that DMSO accounted for only 2.5% of the total reaction volume in each case, and were accompanied by control assays supplemented with 2.5% DMSO only. Owing to the low solubility of the inhibitors, the maximum concentration achievable was 250 μM final. The reaction, initiated by the addition of enzyme (accounting for 5% total reaction volume), was monitored at 400 nm to measure the generation of retinal from retinol. The rate of retinal generation was determined as the change in milli-absorbance units per minute (mAbs/min).

Multi-well assay plates (96) were preloaded with DMSO (2.5% final reaction volume), containing inhibitor where required. For each plate a master mix was prepared consisting of sufficient buffered substrate solution (185 μl per reaction) and SS (5% total reaction volume) for the required number of reactions and immediately aliquoted into the desired wells. The contents of each well were mixed by aspiration and the plate loaded into a multi-well plate reader set at 37° C. The generation of retinal was monitored at 400 nm over 30 minutes, during which time the reaction reached maximum velocity under uninhibited conditions after 600 seconds and began to decelerate after 1200 seconds in each case tested. These parameters were therefore used for the determination of reaction rate in every case presented.

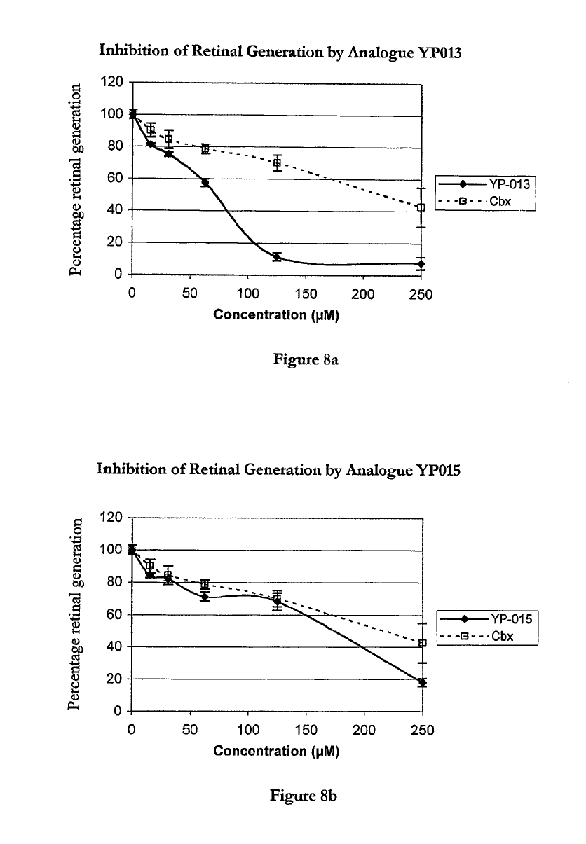

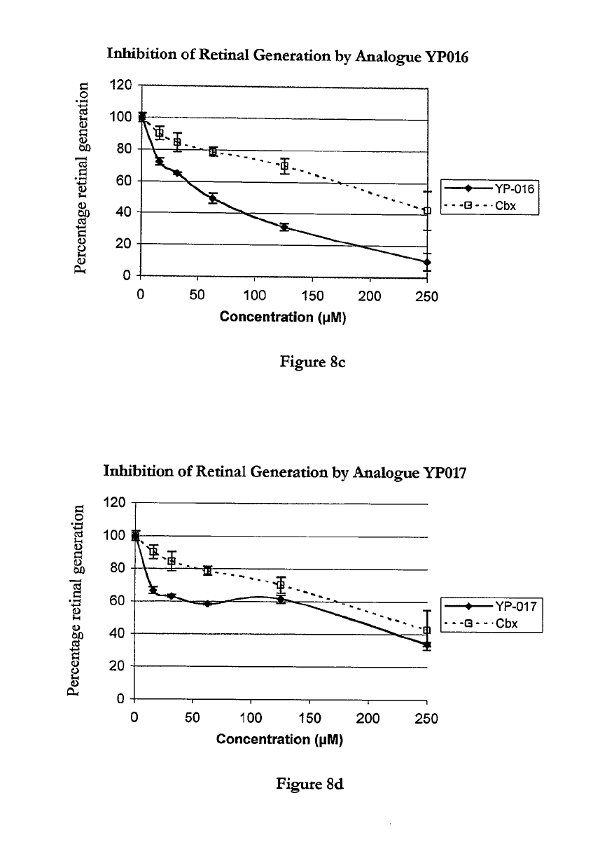

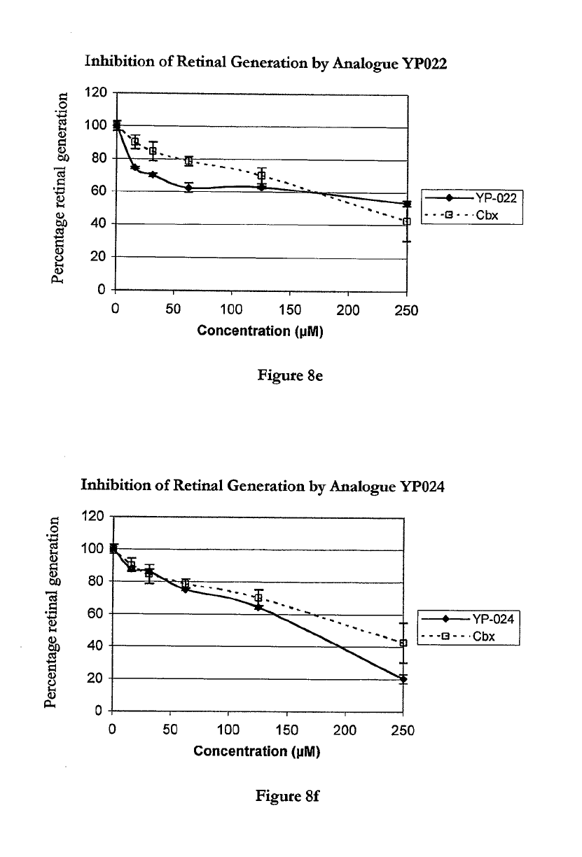

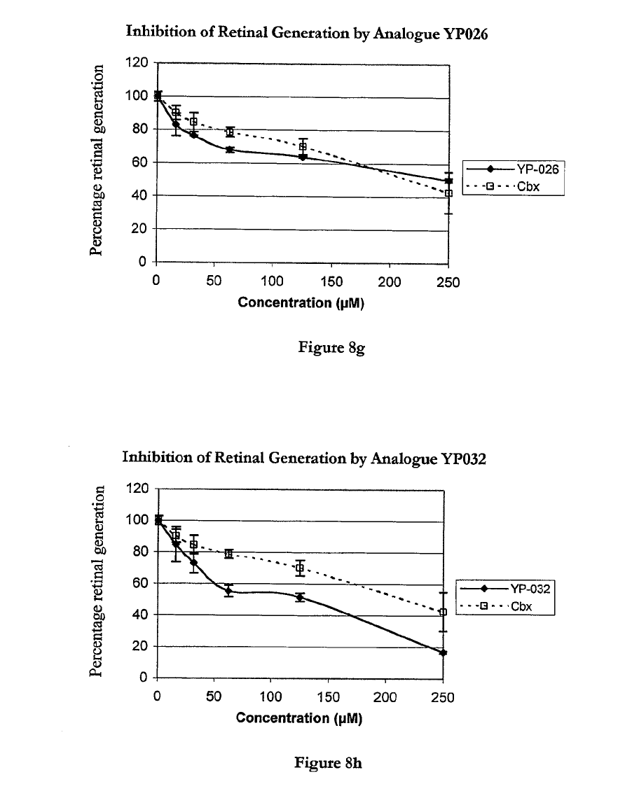

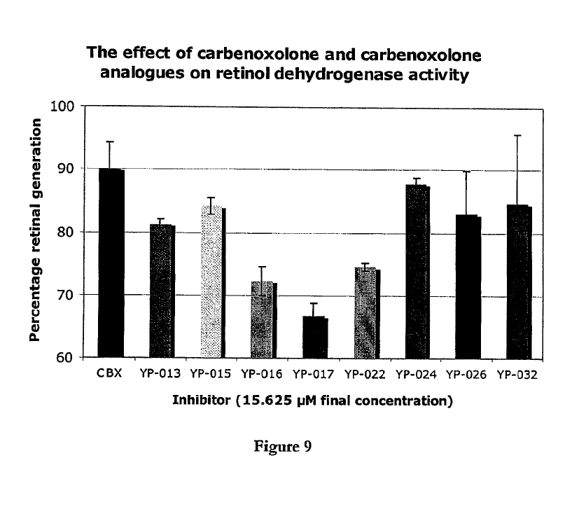

E. coli RIL (-hRoDH-E2) was used as a control and demonstrated no increase in absorbance after 30 minutes (data not shown), demonstrating that retinal is not generated in the absence of hRoDH-E2 expression. The addition of DMSO (2.5%) did not result in a change in absorbance in the absence of recombinant hRoDH-E2. DMSO was found to inhibit enzyme activity in the E. coli RIL (+hRoDH-E2) SS at concentrations above 2.5%, therefore all inhibitors were added in a total volume of 2.5% DMSO in each case, and directly compared to control reactions containing an equal volume of DMSO. FIGS. 8a-h illustrate the effect of eight 18β-glycyrrhetinic acid derivatives of the present invention (YP013, YP015, YP016, YP017, YP022, YP024, YP026 and YP032) compared to carbenoxolone, at increasing concentrations of the inhibitors, on the rate of hRoDH-E2 activity in E. coli RIL (+hRoDH-E2) SS. The effect of all nine inhibitors, at a concentration of 250 μM, on hRoDH-E2 activity is summarised in FIG. 9.

Retinol dehydrogenase activity in E. coli RIL (+hRoDH-E2) SS was demonstrated. This can be attributed to the presence of the hRoDH-E2 expression vector, as E. coli RIL control cells, not harbouring the expression vector, showed no activity. Carbenoxolone is known to inhibit retinol dehydrogenase activity and this was verified by the ability of carbenoxolone to reduce the generation of retinal from retinol, as determined by measuring the absorbance at 400 nm. Control reactions included in the assay demonstrated no non-enzymatic conversion of retinol to retinal, either in the presence of absence of carbenoxolone or DMSO. It can therefore be concluded that the decrease in the generation of retinal in enzyme reactions containing carbenoxolone are due to the inhibition of recombinant hRoDH-E2 activity. All of the eight 18β-glycyrrhetinic acid derivatives of the present invention tested showed similar or greater inhibition of recombinant hRoDH-E2 compared to carbenoxolone.

1. A compound having the formula I: 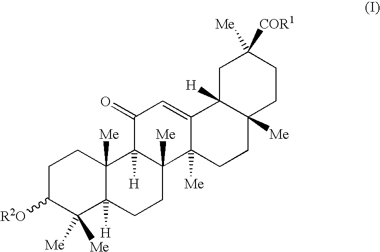
or a pharmaceutically acceptable salt thereof, wherein
R¹ is —OR^{a} or —N(R^{a})₂;
R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;
R² is 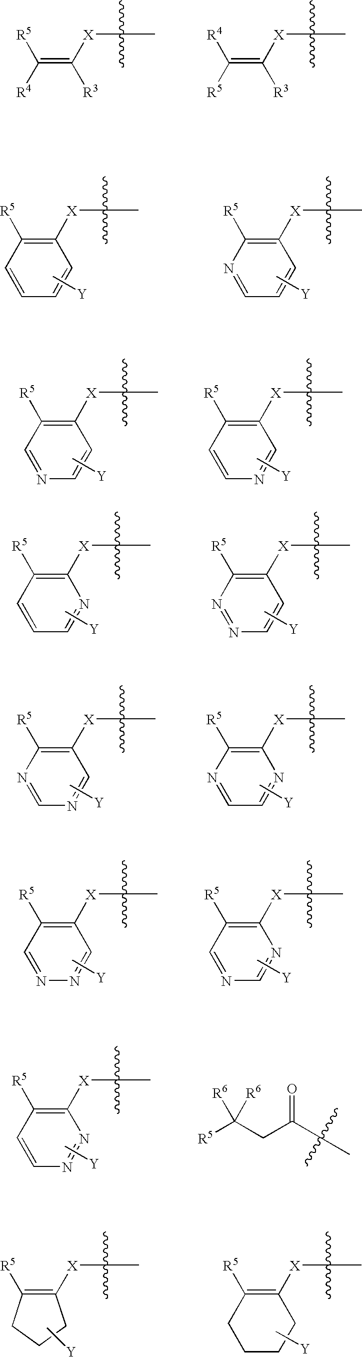 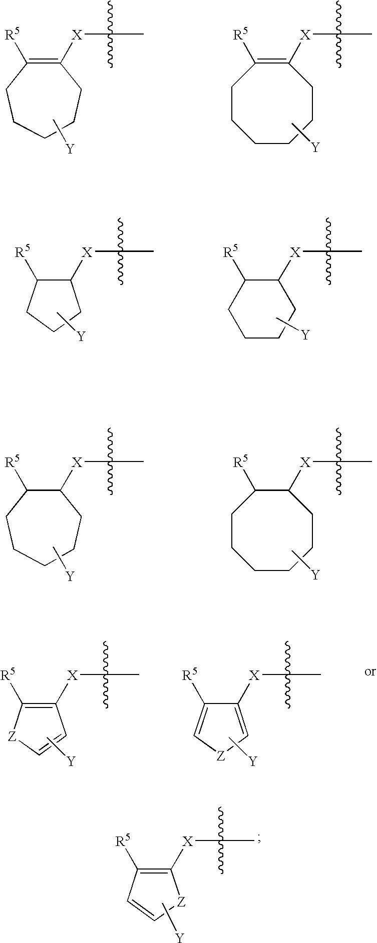
R³ and R⁴ are independently hydrogen, or an unsubstituted, straight-chained or branched alkyl group which contains 1, 2, 3 or 4 carbon atoms;
R⁵ is —OH, —CO₂H, —CO₂R⁶, —SO₃H, or —PO₃H₂;
R⁶ is an unsubstituted, straight-chained or branched alkyl group which contains 1, 2, 3 or 4 carbon atoms;
X is —CO— or —CH₂—;
Y is —H, —F, —Cl, —Br, —I, -Me, or —OMe; and
Z is —NH—, —O—, or —S—;
provided that:
when R¹ is —ONa, R² is not 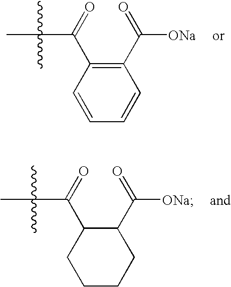
when R¹ is —OMe, R² is not 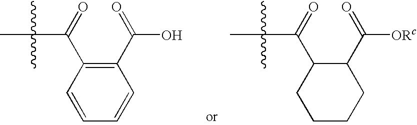
where R^{c} is H or Me; and
when R¹ is —OH, R² is not 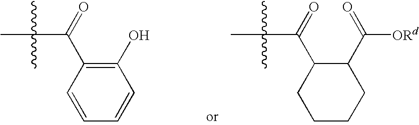
where R^{d} is H or “hexyl; and
when R¹ is —O-“hexyl, R² is not 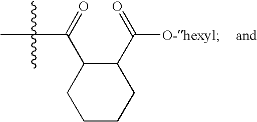
when R¹ is —OH or 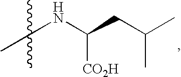
R² is not

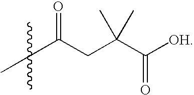

2. A compound as claimed in claim 1, having the formula IA:

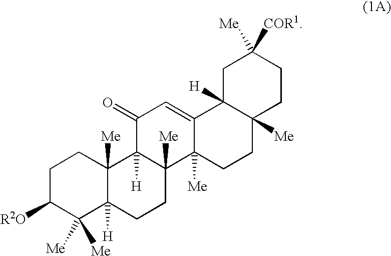

3. A compound as claimed in claim 1, wherein R¹ is:
(a) OH; or
(b) —OMe; or
(c) —OR^{a}; or
(d) —OR^{a}, and wherein R^{a} is an unsubstituted, straight-chained or branched alkyl, alkenyl or alkynyl group which contains 1, 2, 3, 4, 5 or 6 carbon atoms; or
(e) —OR^{a}, and wherein R^{a} is a substituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton; or
(f) —OR^{a}, and wherein R^{a} is a substituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton, and wherein R^{a} is substituted with —OH, —NH₂, —NHMe, —NHEt, or —CO₂H; or
(g) —NH₂, —NHMe, —NMe₂, —NHEt or —NEt₂; or
(h) —NMe₂.

4. A compound as claimed in claim 1, wherein R² is: 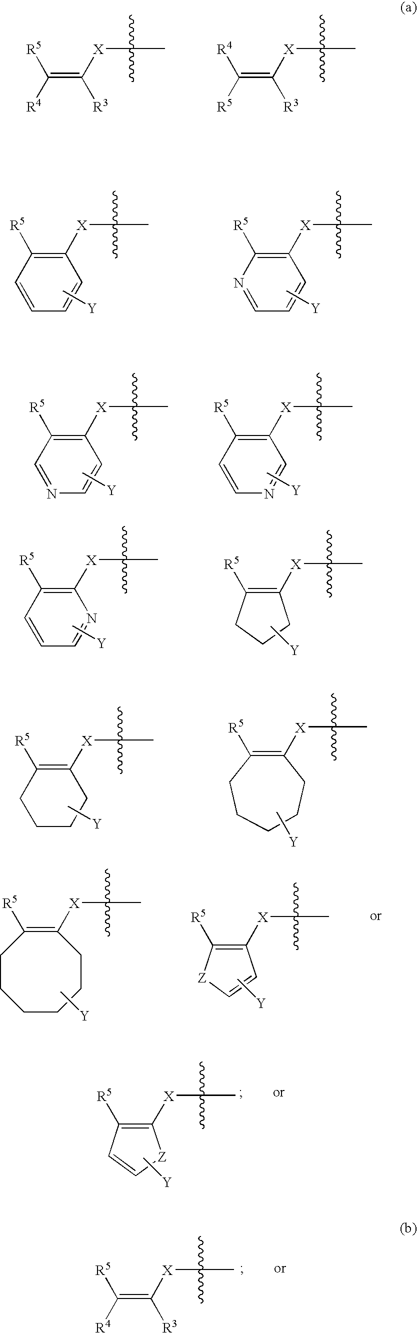 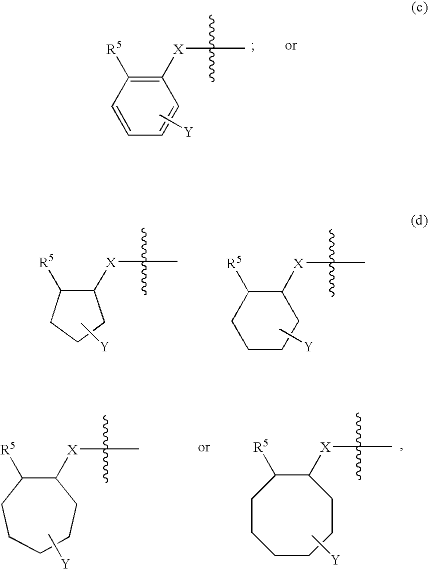
and wherein the compound is one cis-enantiomer, one trans-enantiomer, a mixture of two cis-enantiomers, a mixture of two trans-enantiomers, or a mixture of two cis- and two trans-enantiomers; or 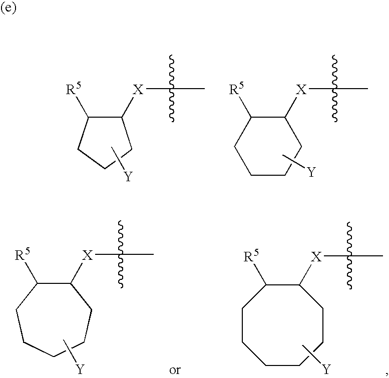
and wherein the compound is one cis-enantiomer, or a mixture of two cis-enantiomers.

5. A compound as claimed in claim 1, wherein:
(a) R³ and/or R⁴ is —H or -Me; or
(b) R⁵ is —OH, —CO₂H or —CO₂R⁶; or
(c) X is —CO—; or
(d) Y is hydrogen or fluorine; or
(e) Y is hydrogen; or
(f) Y is fluorine.

6. A compound as claimed in claim 1, having the structure:

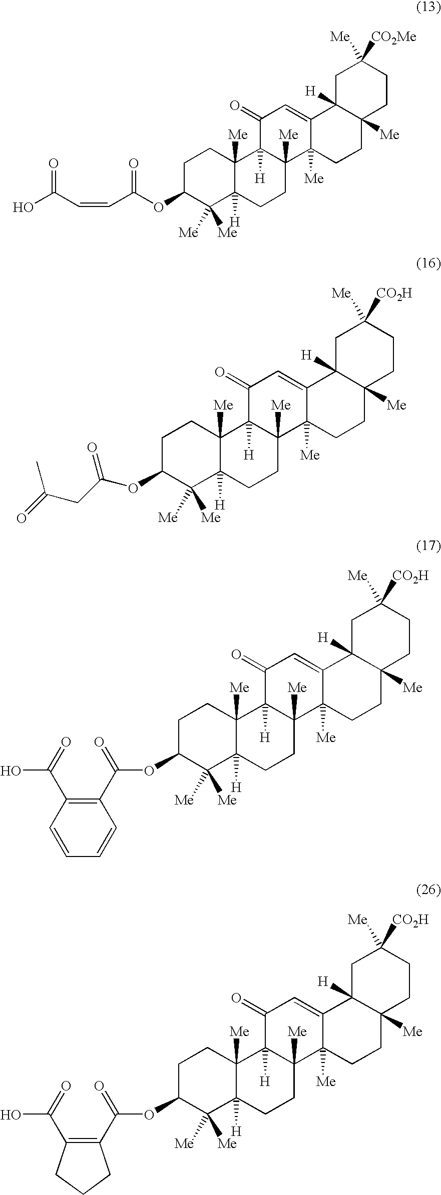

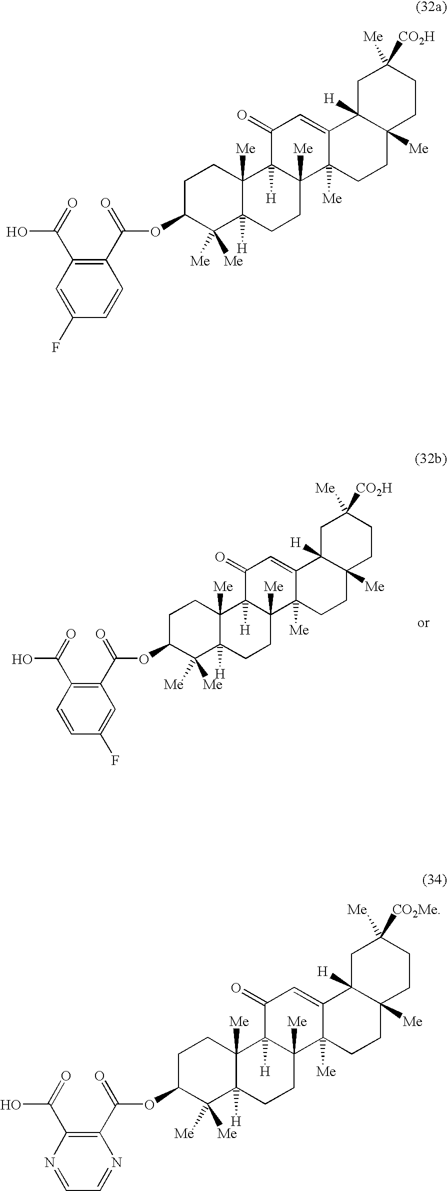

7. A pharmaceutical composition comprising a compound as claimed in claim 1, and a pharmaceutically acceptable excipient, carrier or diluent.

8. A compound as claimed in claim 1, for:
(a) use in medicine; or
(b) lowering the endogenous level or activity of retinoic acid in a cell; or
(c) lowering the endogenous level or activity of retinoic acid in a hyperproliferative cell or a cell suffering from photoageing; or
(d) lowering the endogenous level or activity of retinoic acid in a cell to an extent that cell proliferation is reduced or prevented, and/or to an extent that cell differentiation is activated, enhanced or induced; or
(e) interfering with the biosynthesis of retinoic acid; or
(f) inhibiting an enzyme; or
(g) inhibiting an enzyme in vitro, ex vivo or in vivo; or
(h) antagonising or inhibiting a retinol dehydrogenase (RDH); or
(i) antagonising or inhibiting a retinol dehydrogenase (RDH), wherein the retinol dehydrogenase (RDH) is RoDH1, RoDH2, RoDH3, RoDH4, CRAD1, CRAD2, RDH5, retSDR1 or hRoDH-E2 (official gene symbol DHRS9); or
(j) reducing or preventing cell proliferation; or
(k) activating, enhancing or inducing cell differentiation; or
(l) treating or preventing photoageing in a patient; or
(m) treating or preventing a hyperproliferative disorder in a patient; or
(n) treating or preventing a hyperproliferative disorder in a patient, wherein the hyperproliferative disorder is psoriasis, acne vulgaris, acne rosacea, actinic keratosis, solar keratoses, squamous cell carcinoma in situ, basal cell carcinoma, the ichthyoses, hyperkeratoses, disorders of keratinisation such as Darriers disease, palmoplantar keratodermas, pityriasis rubra pilaris, epidermal naevoid syndromes, erythrokeratoderma variabilis, epidermolytic hyperkeratoses, non-bullous ichthyosiform erythroderma, cutaneous lupus erythematosus, lichen planus, cancer, skin cancer, melanoma or dermatofibroma; or
(o) treating or alleviating the symptoms of a patient suffering from a disorder, wherein the disorder is a retinoid-sensitive disorder treatable by administration of retinoid, or wherein the disorder is a retinoid-sensitive disorder whose symptoms are alleviatable by administration of retinoid, or wherein the disorder corresponds to a side effect of the administration of pharmacological levels of retinoid; or
(p) treating or alleviating the symptoms of a patient suffering from a disease characterised by ectopic, over- or otherwise abnormal expression of a retinoic acid receptor response element (RARE) responsive gene, a vitamin D response element (VDRE) responsive gene, a thyroid hormone receptor response element responsive gene, or a peroxisome proliferator-activated receptor (PPAR) response element responsive gene; or
(q) treating or alleviating the symptoms of a patient suffering from a disease characterised by an imbalance between proliferation and differentiation; or
(r) treating a patient suffering from a disease, disorder or condition, wherein the disease, disorder or condition is a viral infection, HPV infection, HIV infection, HSV infection, HCV infection, EBV infection, warts, postoperative scarring, hypertrophic or keloid scarring, a disorder of melanogenesis, a disorder of pigmentation, enhanced or compromised epidermal barrier function, a disorder of bone growth, bone fracture, osteoporosis, hyperlipidaemia, hepatotoxicity, cirrhosis, hepatitis infection, cutaneous irritation, alopecia, a disorder of fertility, a disorder of spermatogenesis, a disorder of egg implantation, depression, seasonal affective disorder, atherosclerosis, or a disorder of angiogenesis.

9. A method of treating or alleviating the symptoms of a patient suffering from a disorder or disease, wherein:
(a) the disorder is a retinoid-sensitive disorder treatable by administration of retinoid, or
(b) the disorder is a retinoid-sensitive disorder whose symptoms are alleviatable by administration of retinoid, or
(c) the disorder corresponds to a side effect of the administration of pharmacological levels of retinoid, or
(d) the disease is characterised by ectopic, over- or otherwise abnormal expression of a retinoic acid receptor response element (RARE) responsive gene, a vitamin D response element (VDRE) responsive gene, a thyroid hormone receptor response element responsive gene, or a peroxisome proliferator-activated receptor (PPAR) response element responsive gene,
which method comprises administering a therapeutically effective amount of a compound to the patient, wherein the compound has the formula I: 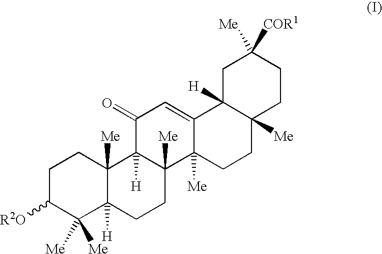
or a pharmaceutically acceptable salt thereof, wherein
R¹ is —OR^{a} or —N(R^{a})₂;
R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;
R² is 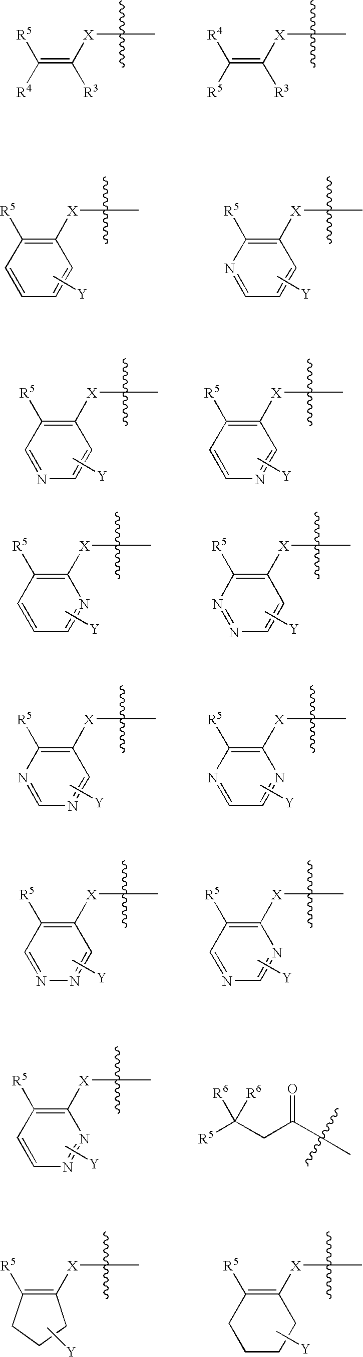 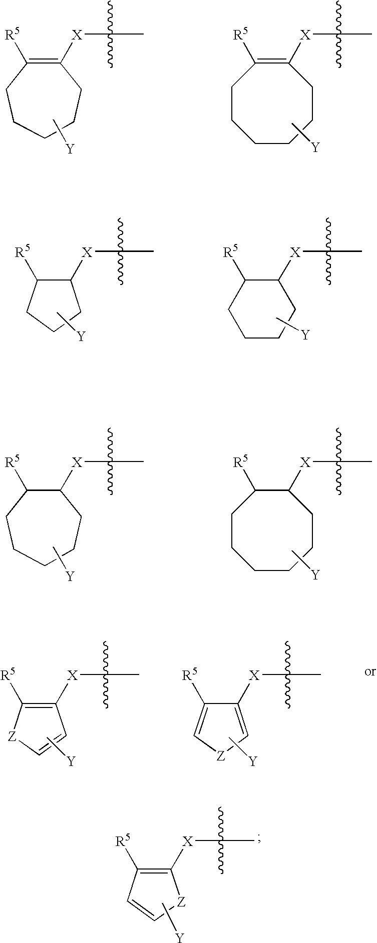
R³ and R⁴ are independently hydrogen, or an unsubstituted, straight-chained or branched alkyl group which contains 1, 2, 3 or 4 carbon atoms;
R⁵ is —OH, —CO₂H, —CO₂R⁶, —SO₃H, or —PO₃H₂;
R⁶ is an unsubstituted, straight-chained or branched alkyl group which contains 1, 2, 3 or 4 carbon atoms;
X is —CO— or —CH₂—;
Y is —H, —F, —Cl, —Br, —I, -Me, or —OMe; and
Z is —NH—, —O—, or —S—.

10. A method of treating or preventing photoageing in a patient, which method comprises administering a therapeutically or prophylactically effective amount of a compound to the patient, wherein the compound has the formula I: 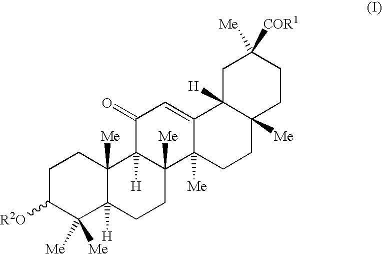
or a pharmaceutically acceptable salt thereof, wherein
R¹ is —OR^{a} or —N(R^{a})₂;
R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;
R² is 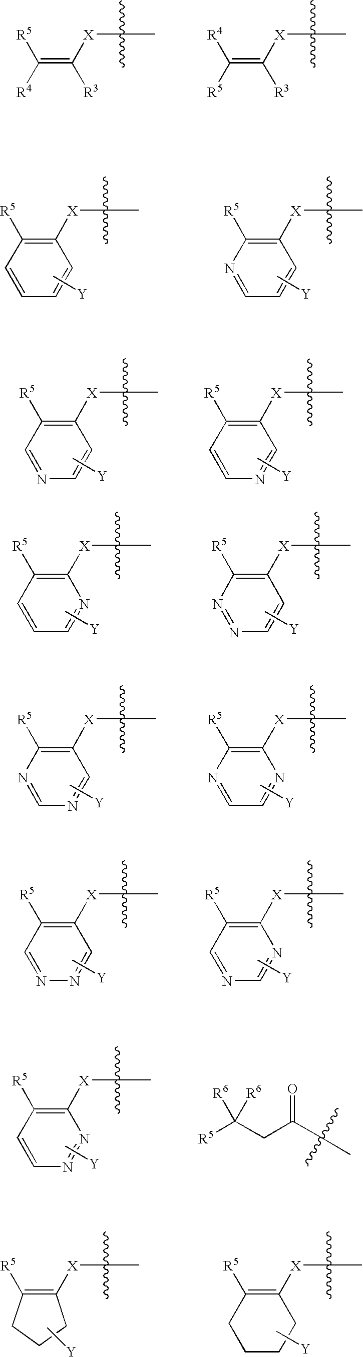 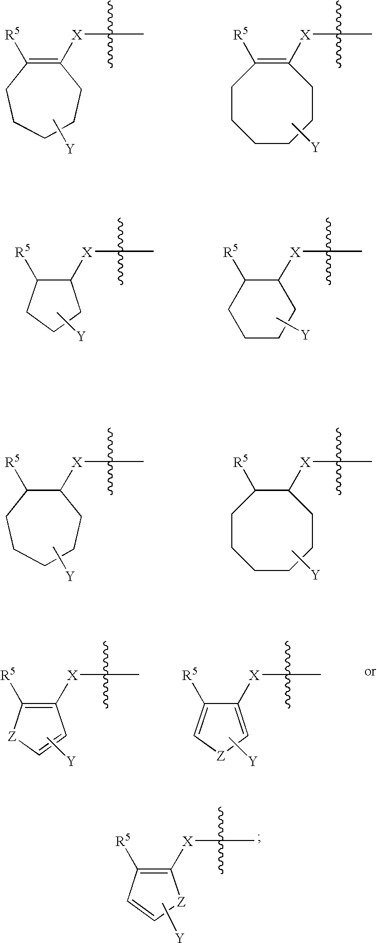
R³ and R⁴ are independently hydrogen, or an unsubstituted, straight-chained or branched alkyl group which contains 1, 2, 3 or 4 carbon atoms;
R⁵ is —OH, —CO₂H, —CO₂R⁶, —SO₃H, or —PO₃H₂;
R⁶ is an unsubstituted, straight-chained or branched alkyl group which contains 1, 2, 3 or 4 carbon atoms;
X is —CO— or —CH₂—;
Y is —H, —F, —Cl, —Br, —I, -Me, or —OMe; and
Z is —NH—, —O—, or —S—.

11. A method of treating or preventing a hyperproliferative disorder in a patient, which method comprises administering a therapeutically or prophylactically effective amount of a compound to the patient, wherein the compound has the formula I: 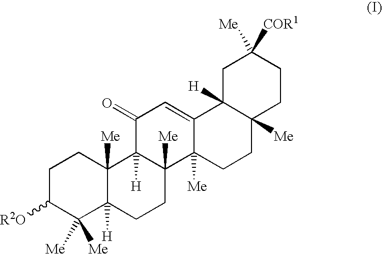
or a pharmaceutically acceptable salt thereof, wherein
R¹ is —OR^{a} or —N(R^{a})₂;
R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;
R² is 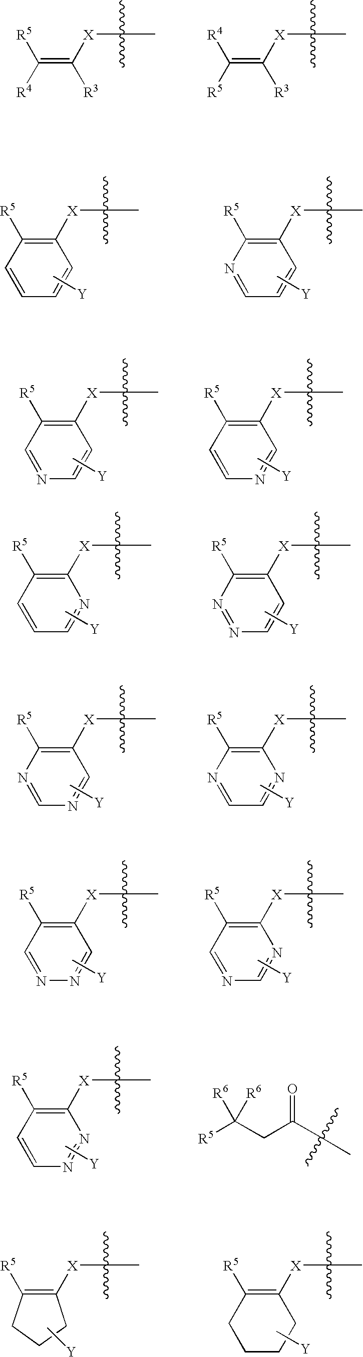 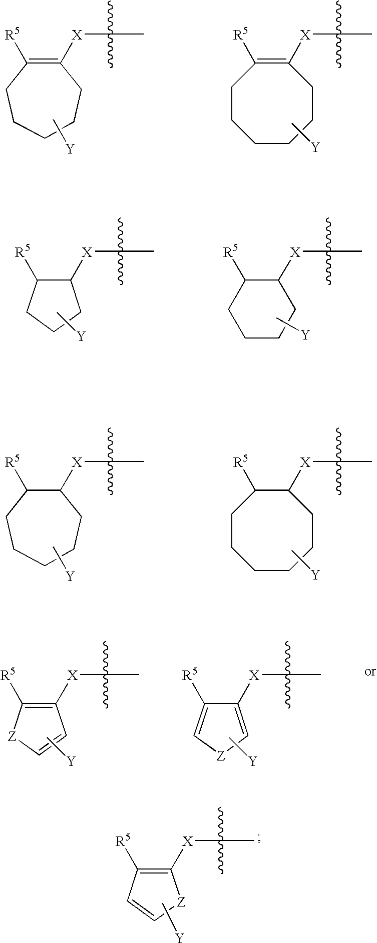
R³ and R⁴ are independently hydrogen, or an unsubstituted, straight-chained or branched alkyl group which contains 1, 2, 3 or 4 carbon atoms;
R⁵ is —OH, —CO₂H, —CO₂R⁶, —SO₃H, or —PO₃H₂;
R⁶ is an unsubstituted, straight-chained or branched alkyl group which contains 1, 2, 3 or 4 carbon atoms;
X is —CO— or —CH₂—;
Y is —H, —F, —Cl, —Br, —I, -Me, or —OMe; and
Z is —NH—, —O—, or —S—;
provided that:
when R¹ is —ONa, R² is not 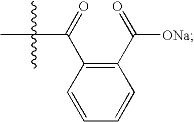
and
when R¹ is —OH, R² is not

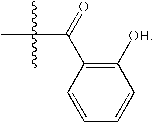

12. A method as claimed in claim 11, wherein the compound has the structure:

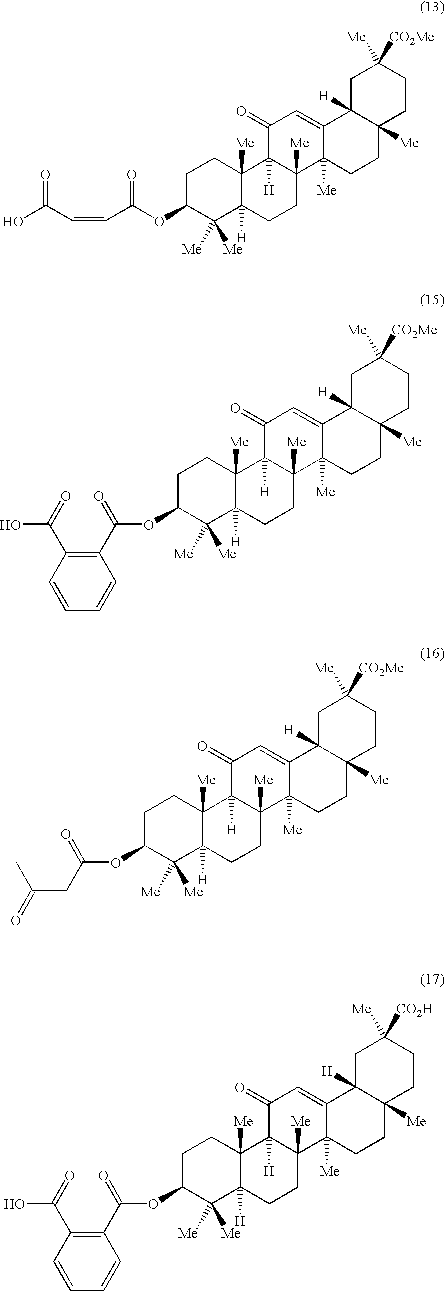

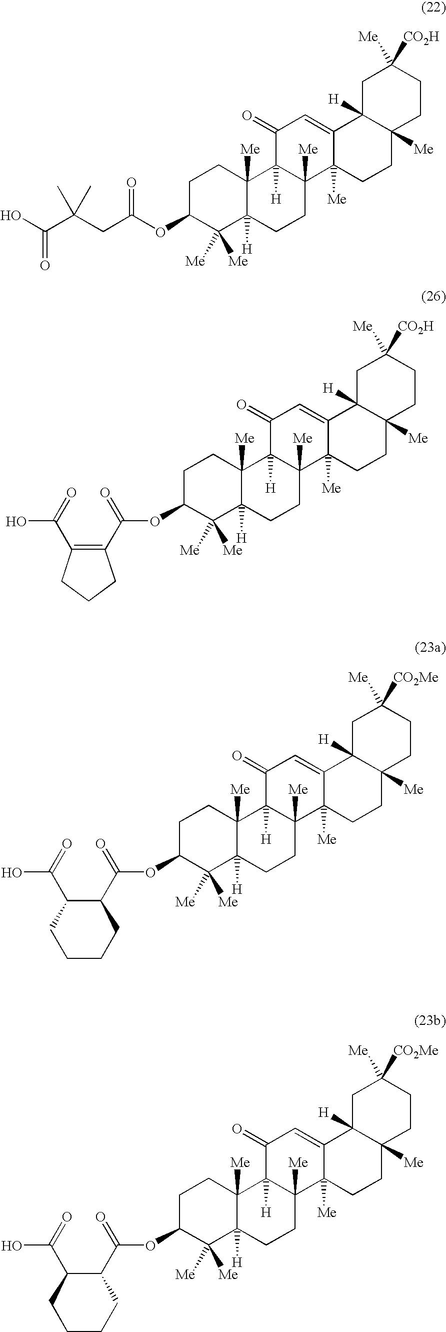

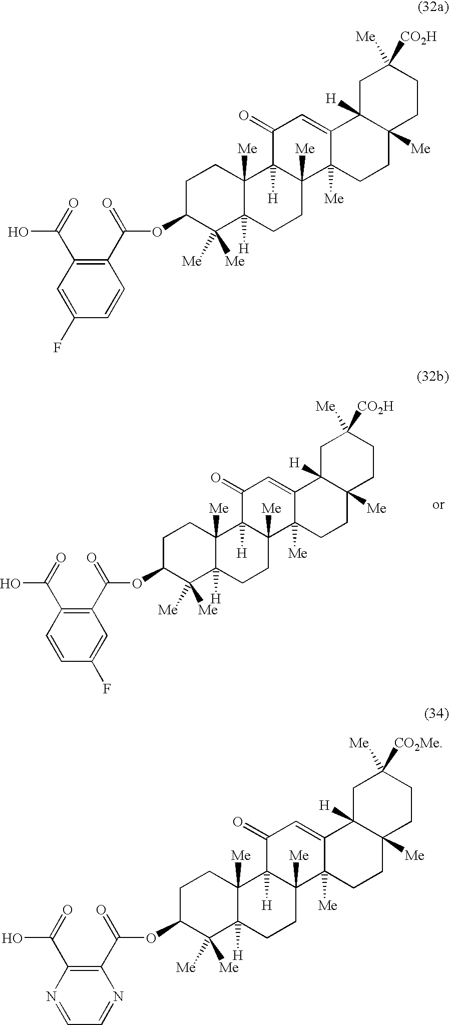

13. A method as claimed in claim 11, wherein the hyperproliferative disorder is psoriasis, acne vulgaris, acne rosacea, actinic keratosis, solar keratoses, squamous cell carcinoma in situ, basal cell carcinoma, the ichthyoses, hyperkeratoses, disorders of keratinisation such as Darriers disease, palmoplantar keratodermas, pityriasis rubra pilaris, epidermal naevoid syndromes, erythrokeratoderma variabilis, epidermolytic hyperkeratoses, non-bullous ichthyosiform erythroderma, cutaneous lupus erythematosus, lichen planus, cancer, skin cancer, melanoma or dermatofibroma.

14. A method of treating or preventing a hyperproliferative disorder in a patient, wherein the hyperproliferative disorder is psoriasis, acne vulgaris, acne rosacea, actinic keratosis, solar keratoses, squamous cell carcinoma in situ, basal cell carcinoma, the ichthyoses, hyperkeratoses, disorders of keratinisation such as Darriers disease, palmoplantar keratodermas, pityriasis rubra pilaris, epidermal naevoid syndromes, erythrokeratoderma variabilis, epidermolytic hyperkeratoses, non-bullous ichthyosiform erythroderma, cutaneous lupus erythematosus, lichen planus, skin cancer, melanoma or dermatofibroma, which method comprises administering a therapeutically or prophylactically effective amount of a compound to the patient, wherein the compound has the formula I: 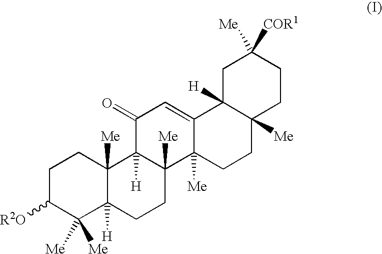
or a pharmaceutically acceptable salt thereof, wherein
R¹ is —OR^{a} or —N(R^{a})₂;
R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;
R² is 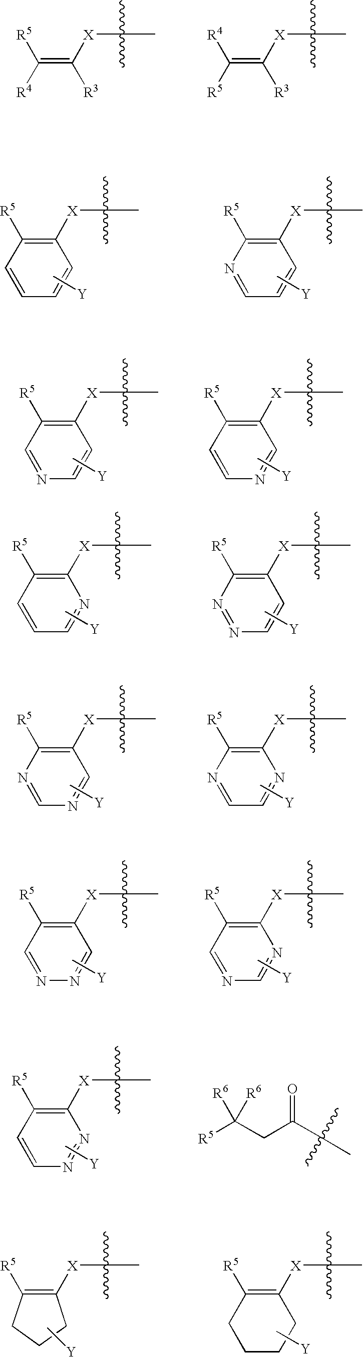 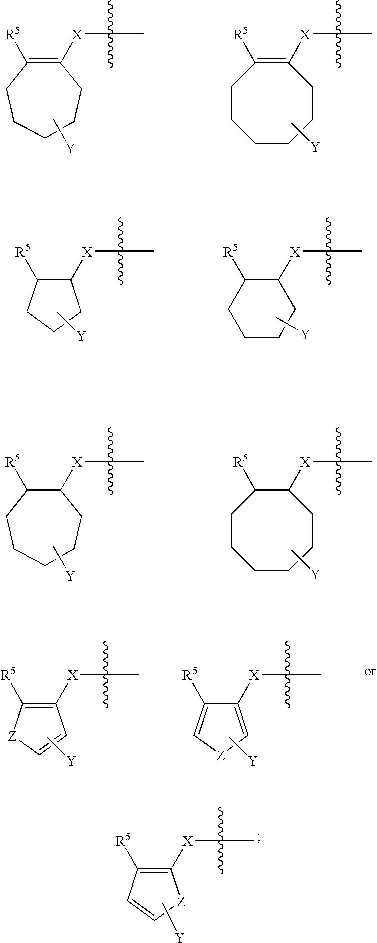
R³ and R⁴ are independently hydrogen, or an unsubstituted, straight-chained or branched alkyl group which contains 1, 2, 3 or 4 carbon atoms;
R⁵ is —OH, —CO₂H, —CO₂R⁶, —SO₃H, or —PO₃H₂;
R⁶ is an unsubstituted, straight-chained or branched alkyl group which contains 1, 2, 3 or 4 carbon atoms;
X is —CO— or —CH₂—;
Y is —H, —F, —Cl, —Br, —I, -Me, or —OMe; and
Z is —NH—, —O—, or —S—.

15. A method as claimed in claim 14, wherein the compound has the structure:

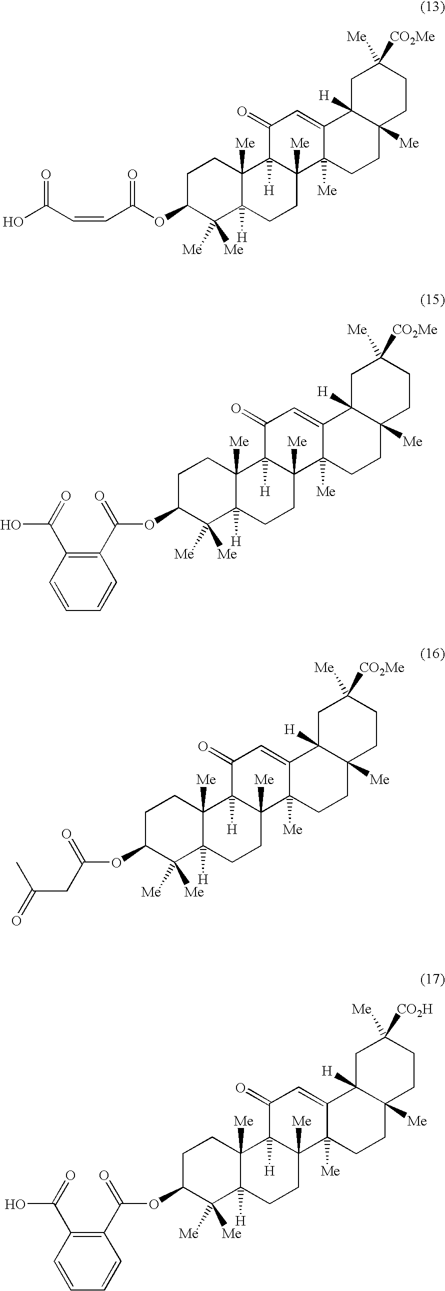

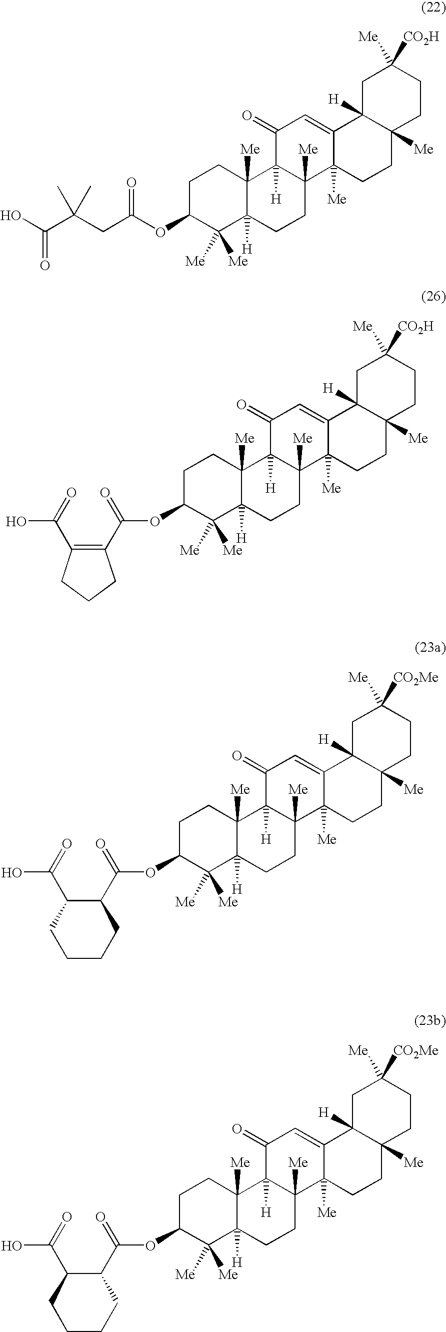

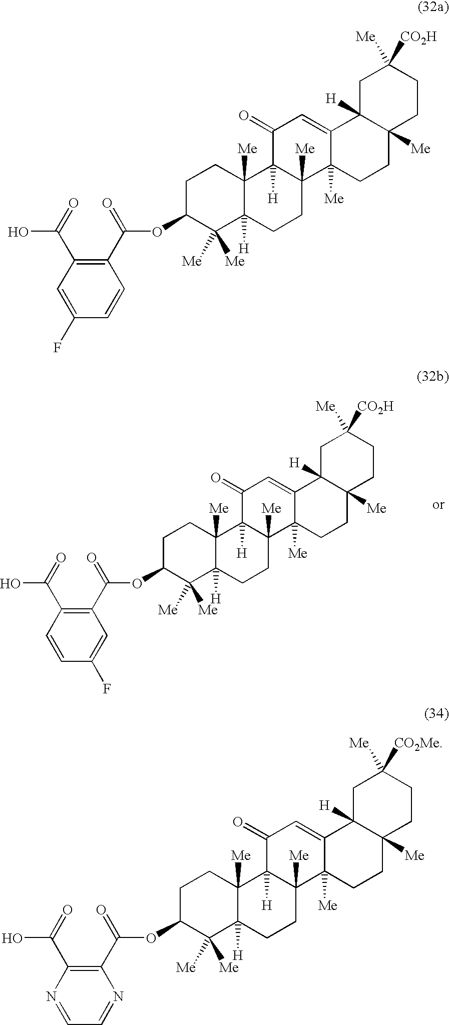

16. A method of reducing or preventing proliferation of a cell in a patient or a method of activating or enhancing a differentiation program in a cell in a patient, which method comprises contacting the cell with a therapeutically or prophylactically effective amount of a compound, wherein the compound has the formula I: 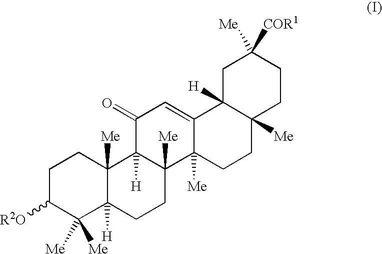
or a pharmaceutically acceptable salt thereof, wherein
R¹ is —OR^{a} or —N(R^{a})₂;
R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;
R² is 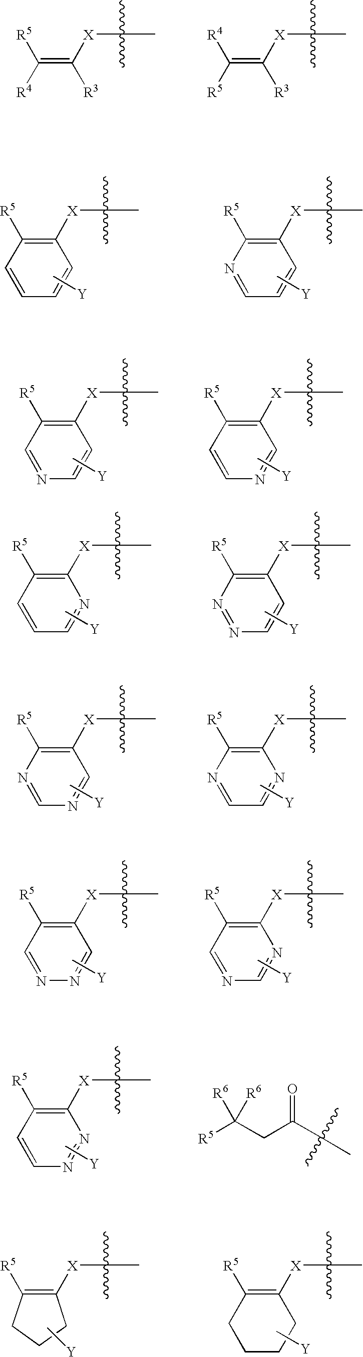 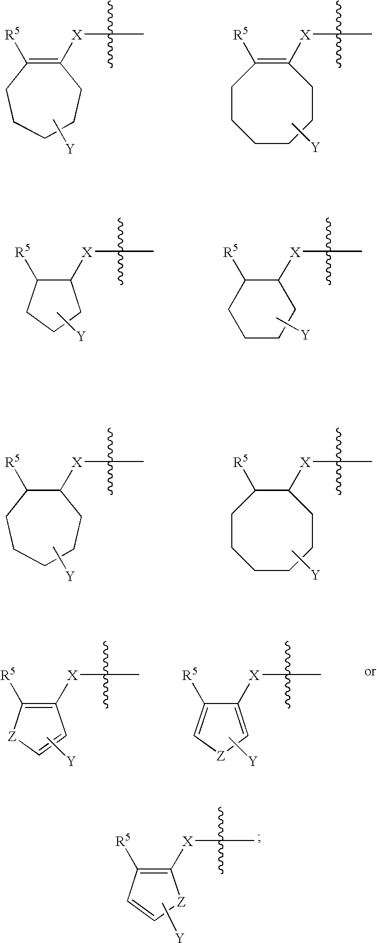
R³ and R⁴ are independently hydrogen, or an unsubstituted, straight-chained or branched alkyl group which contains 1, 2, 3 or 4 carbon atoms;
R⁵ is —OH, —CO₂H, —CO₂R⁶, —SO₃H, or —PO₃H₂;
R⁶ is an unsubstituted, straight-chained or branched alkyl group which contains 1, 2, 3 or 4 carbon atoms;
X is —CO— or —CH₂—;
Y is —H, —F, —Cl, —Br, —I, -Me, or —OMe; and
Z is —NH—, —O—, or —S—.

17. A method of treating or alleviating the symptoms of a patient suffering from a disease characterised by an imbalance between proliferation and differentiation, which method comprises administering a therapeutically effective amount of a compound to the patient, wherein the compound has the formula I: 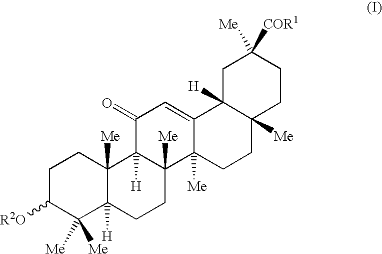
or a pharmaceutically acceptable salt thereof, wherein
R¹ is —OR¹ or —N(R^{a})₂;
R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;
R² is 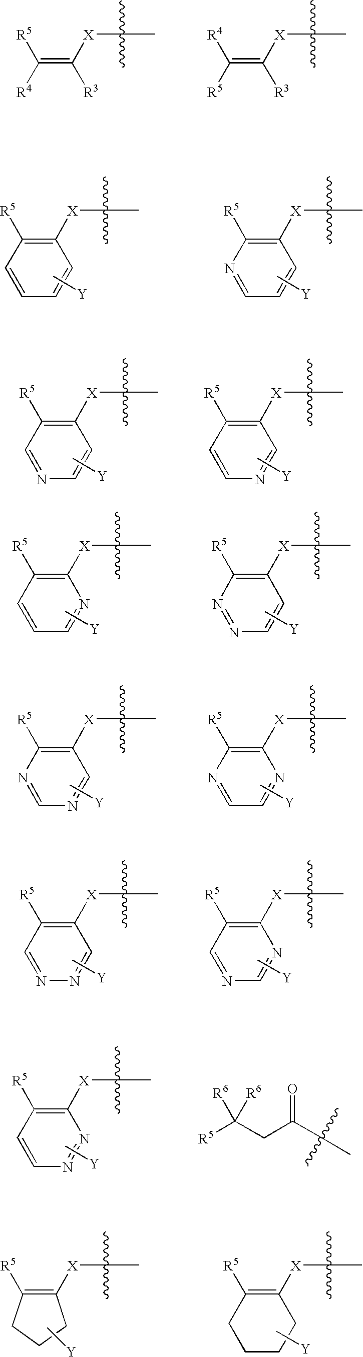 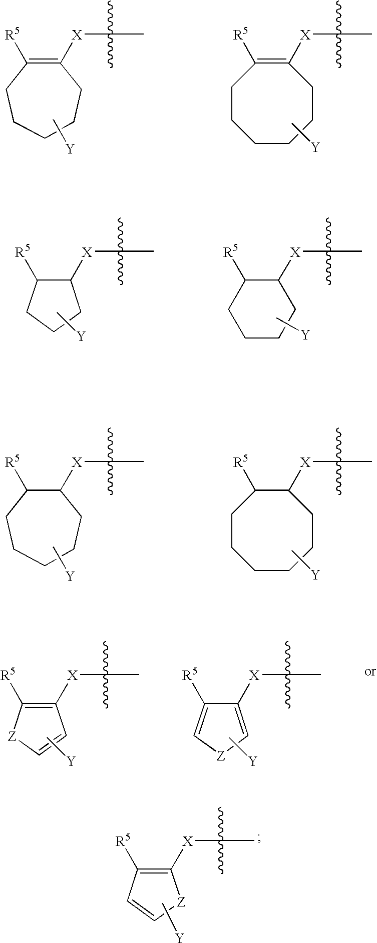
R³ and R⁴ are independently hydrogen, or an unsubstituted, straight-chained or branched alkyl group which contains 1, 2, 3 or 4 carbon atoms;
R⁵ is —OH, —CO₂H, —CO₂R⁶, —SO₃H, or —PO₃H₂;
R⁶ is an unsubstituted, straight-chained or branched alkyl group which contains 1, 2, 3 or 4 carbon atoms;
X is —CO— or —CH₂—;
Y is —H, —F, —Cl, —Br, —I, -Me, or —OMe; and
Z is —NH—, —O—, or —S—.

18. A method of treating or preventing a disease, disorder or condition in a patient, wherein the disease, disorder or condition is a viral infection, HPV infection, HIV infection, HSV infection, HCV infection, EBV infection, warts, postoperative scarring, hypertrophic or keloid scarring, a disorder of melanogenesis, a disorder of pigmentation, enhanced or compromised epidermal barrier function, a disorder of bone growth, bone fracture, osteoporosis, hyperlipidaemia, hepatotoxicity, cirrhosis, hepatitis infection, cutaneous irritation, alopecia, a disorder of fertility, a disorder of spermatogenesis, a disorder of egg implantation, depression, seasonal affective disorder, atherosclerosis, or a disorder of angiogenesis, which method comprises administering a therapeutically or prophylactically effective amount of a compound to the patient, wherein the compound has the formula I: 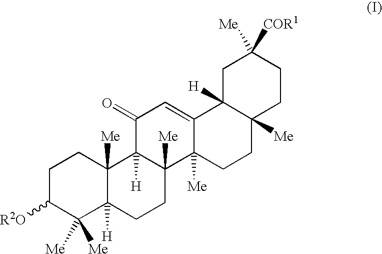
or a pharmaceutically acceptable salt thereof, wherein
R¹ is —OR^{a} or —N(R^{a})₂;
R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;
R² is 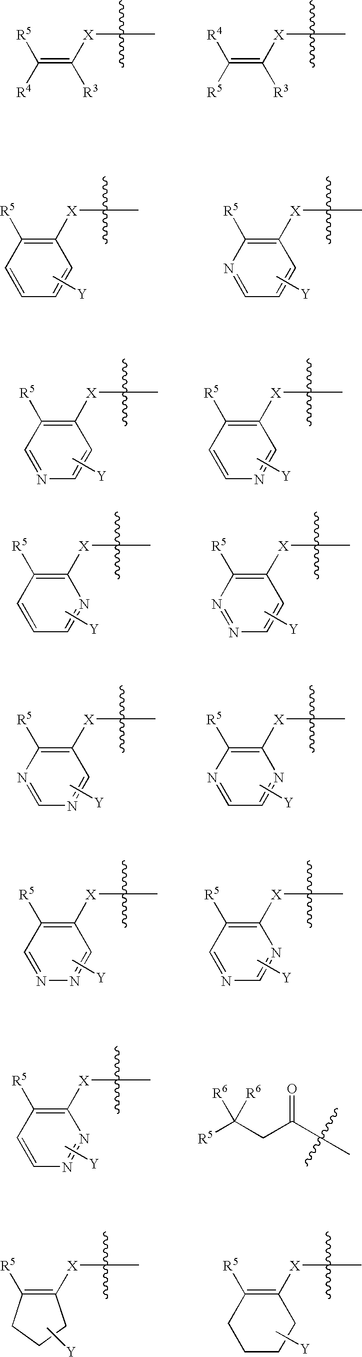 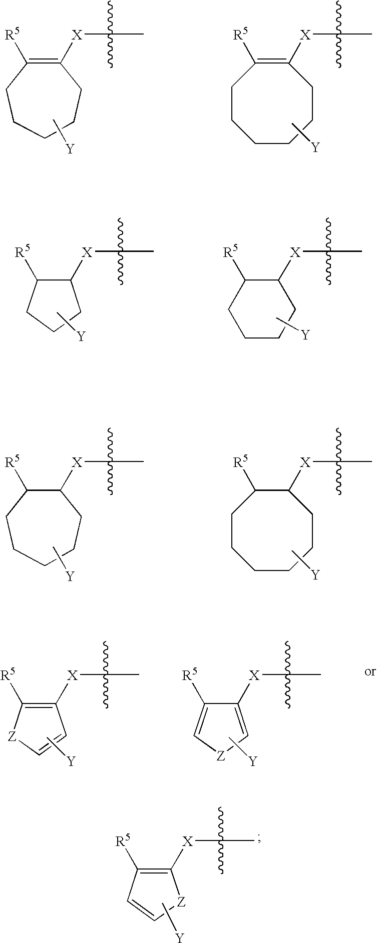
R³ and R⁴ are independently hydrogen, or an unsubstituted, straight-chained or branched alkyl group which contains 1, 2, 3 or 4 carbon atoms;
R⁵ is —OH, —CO₂H, —CO₂R⁶, —SO₃H, or —PO₃H₂;
R⁶ is an unsubstituted, straight-chained or branched alkyl group which contains 1, 2, 3 or 4 carbon atoms;
X is —CO— or —CH₂—;
Y is —H, —F, —Cl, —Br, —I, -Me, or OMe; and
Z is —NH—, —O—, or —S—;
provided that:
when R¹ is —ONa, R² is not 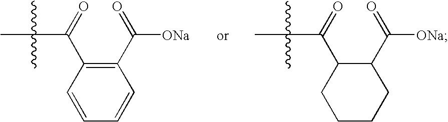
and
when R¹ is —OMe, R² is not 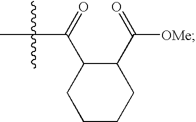
and
when R¹ is —OH, R² is not 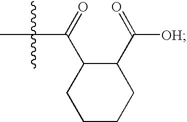
and
when R¹ is —O-“hexyl, R² is not

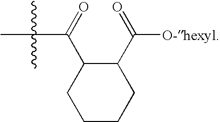

19. A method as claimed in claim 18, wherein the compound has the structure:

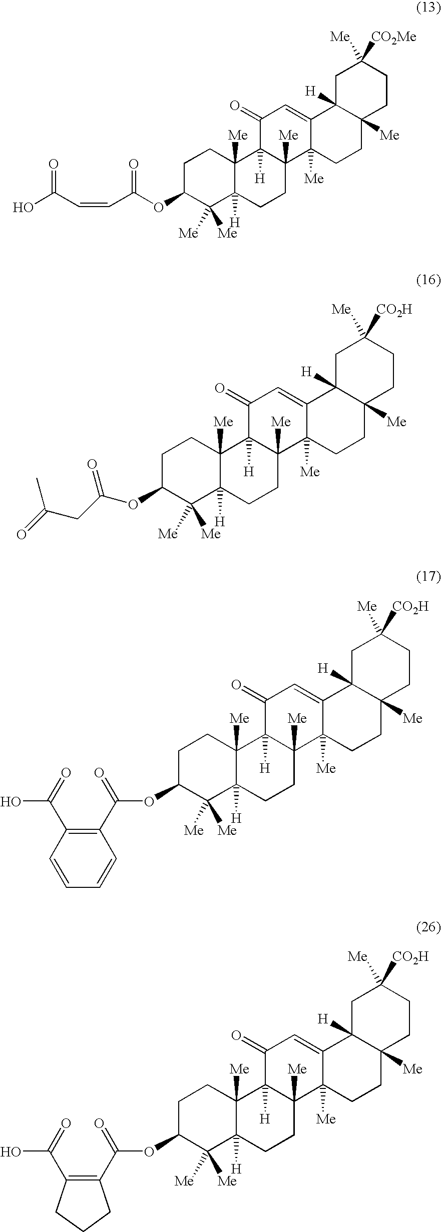

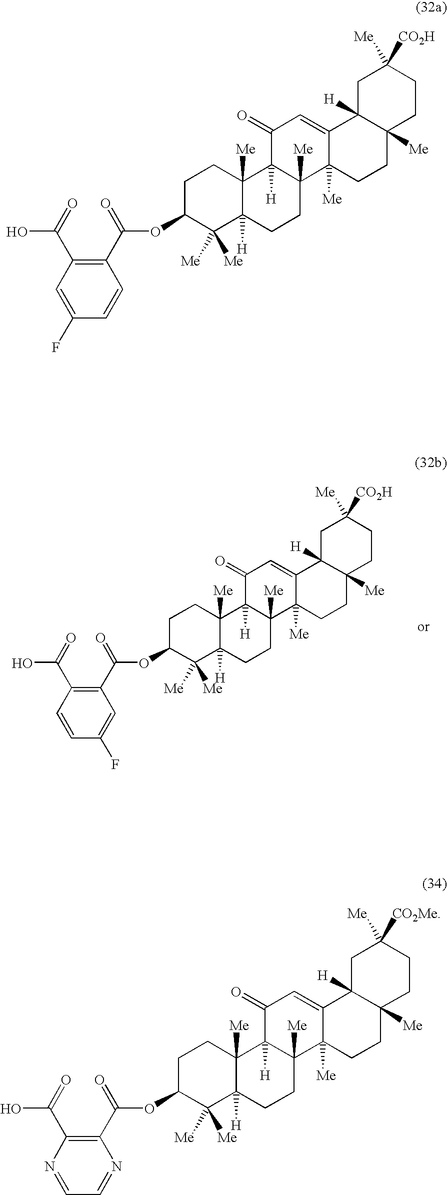

20. A method of treating or preventing a disease, disorder or condition in a patient, wherein the disease, disorder or condition is HPV infection, HIV infection, HCV infection, EBV infection, warts, postoperative scarring, hypertrophic or keloid scarring, a disorder of melanogenesis, a disorder of pigmentation, enhanced or compromised epidermal barrier function, a disorder of bone growth, bone fracture, osteoporosis, hyperlipidaemia, hepatotoxicity, cirrhosis, hepatitis infection, cutaneous irritation, alopecia, a disorder of fertility, a disorder of spermatogenesis, a disorder of egg implantation, depression, seasonal affective disorder, atherosclerosis, or a disorder of angiogenesis, which method comprises administering a therapeutically or prophylactically effective amount of a compound to the patient, wherein the compound has the formula I: 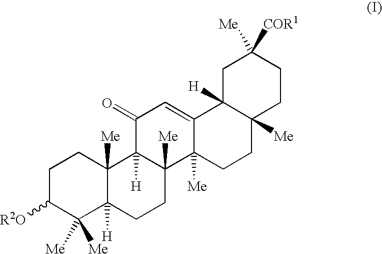
or a pharmaceutically acceptable salt thereof, wherein
R¹ is —OR^{a} or —N(R^{a})₂;
R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;
R² is 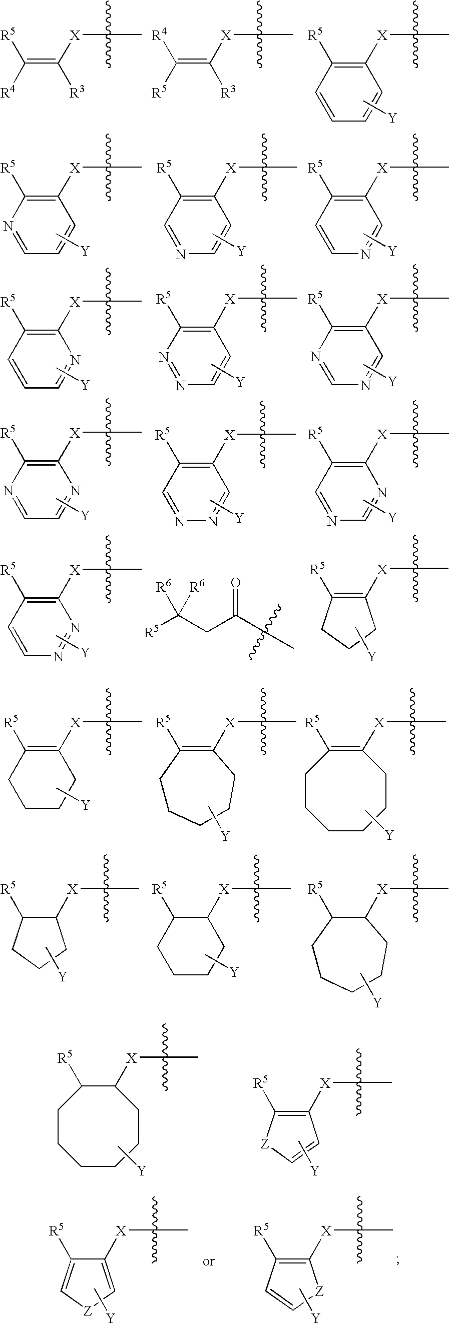
R³ and R⁴ are independently hydrogen, or an unsubstituted, straight-chained or branched alkyl group which contains 1, 2, 3 or 4 carbon atoms;
R⁵ is —OH, —CO₂H, —CO₂R⁶, —SO₃H, or PO₃H₂;
R⁶ is an unsubstituted, straight-chained or branched alkyl group which contains 1, 2, 3 or 4 carbon atoms;
X is —CO— or —CH₂—;
Y is —H, —F, —Cl, —Br, —I, -Me, or —OMe; and
Z is —NH—, —O—, or —S—.

21. A method as claimed in claim 20, wherein the compound has the structure:

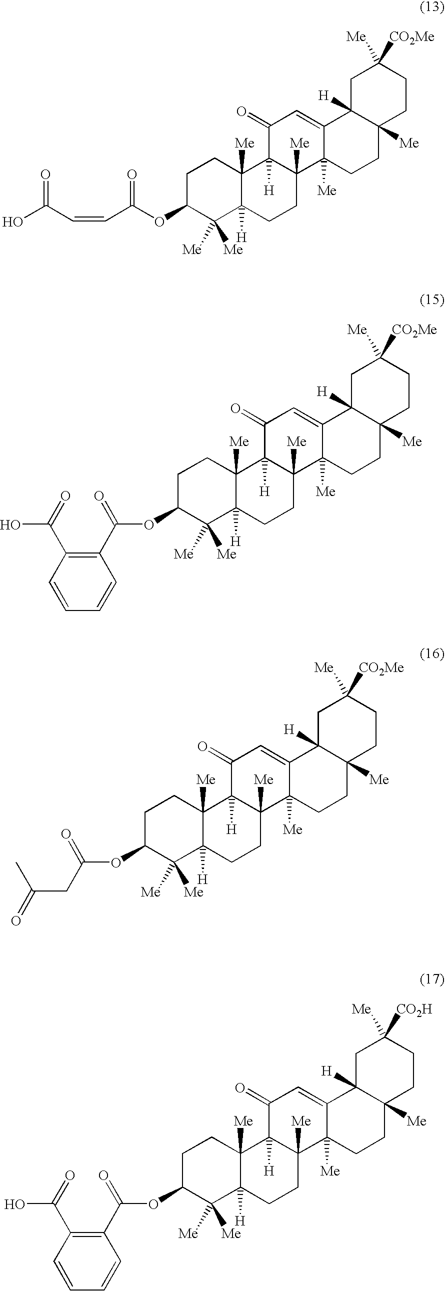

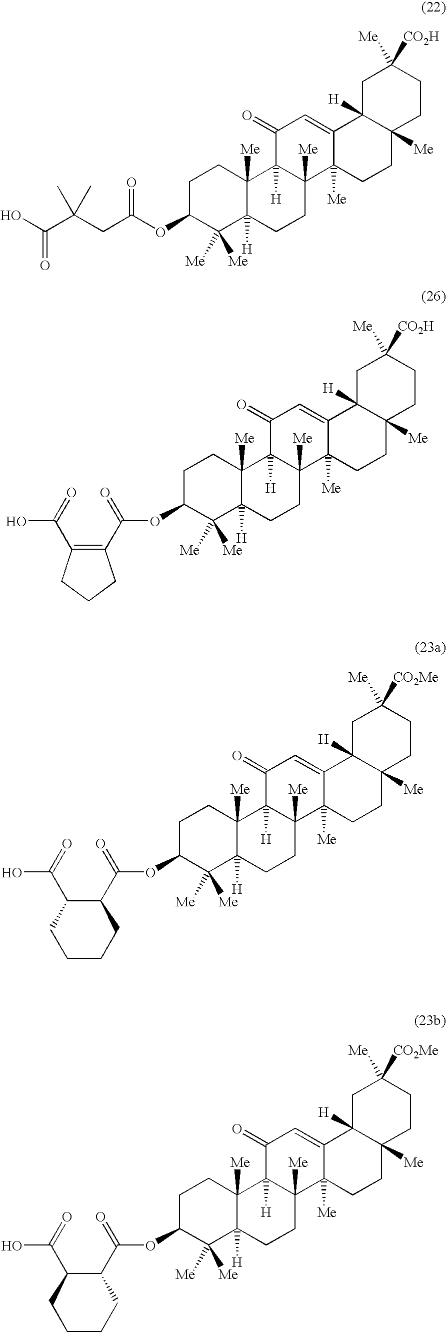

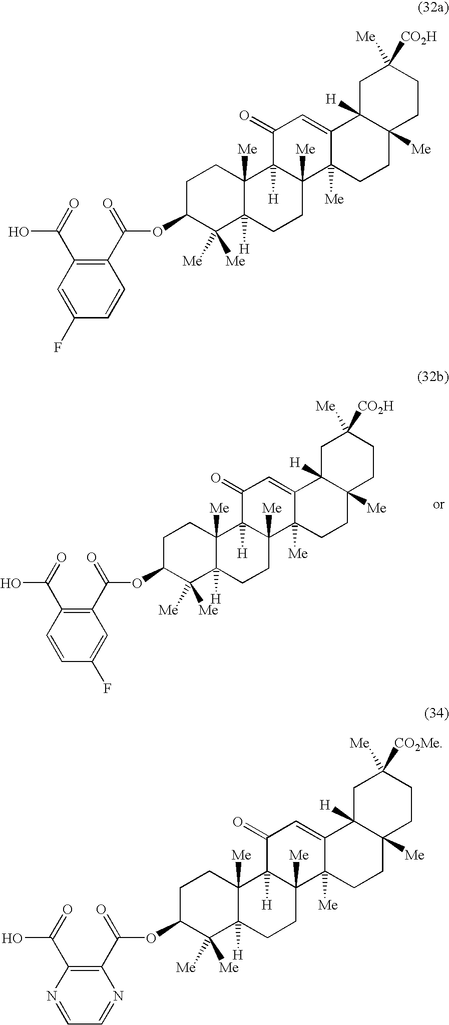

22. A method of inhibiting an enzyme, comprising administering an inhibitory amount of a compound to a patient in need thereof, wherein the enzyme is a retinol dehydrogenase (RDH), and wherein the compound has the formula I: 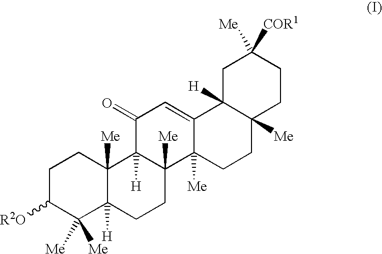
or a pharmaceutically acceptable salt thereof, wherein
R¹ is —OR^{a} or —N(R^{a})₂;
R^{a} is hydrogen, or a substituted or unsubstituted, straight-chained or branched alkyl, alkenyl or alkynyl group which contains 1, 2, 3, 4, 5 or 6 carbon atoms and optionally includes 1, 2 or 3 heteroatoms N, O or S in its carbon skeleton;
R² is 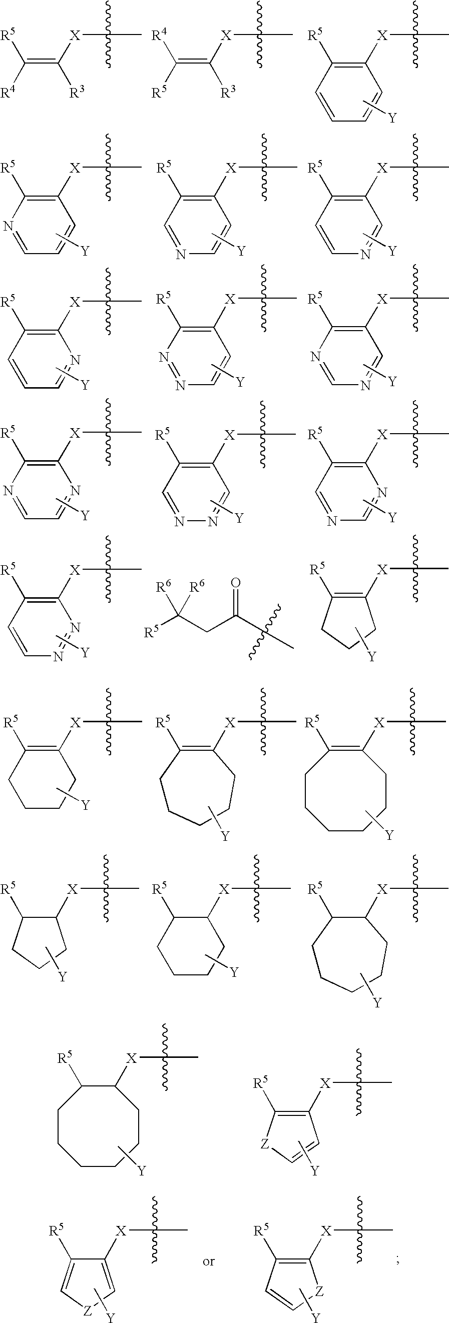
R³ and R⁴ are independently hydrogen, or an unsubstituted, straight-chained or branched alkyl group which contains 1, 2, 3 or 4 carbon atoms;
R⁵ is —OH, —CO₂H, —CO₂R⁶, —SO₃H, or —PO₃H₂;
R⁶ is an unsubstituted, straight-chained or branched alkyl group which contains 1, 2, 3 or 4 carbon atoms;
X is —CO— or —CH₂—;
Y is —H, —F, —Cl, —Br, —I, -Me, or —OMe; and
Z is —NH—, —O—, or —S—.

23. A method of synthesising a compound as claimed in claim 1, using 18β-glycyrrhetinic acid as a starting material.

24. A method as claimed in claim 23, wherein:
(a) the 3-hydroxyl group of 18β-glycyrrhetinic acid is esterified or alkylated; or
(b) the 20-carboxyl group of 18β-glycyrrhetinic acid is esterified or converted into an amide group.
